(19) United States
(12) Patent Application Publication
(10) Pub. No.: US 2023/0279002 A1
(43) Pub. Date: Sep. 7, 2023

(54) Polymorphs of Crystalline Forms of 3,10-dimethoxy-5,8,13,13a-tetrahydro-6H-isoquinolino[3,2-a]isoquinolin-9-yl 3-fluorobenzenesulfonate and Salts Thereof

(71) Applicant: CVI Pharmaceuticals Limited, Grand Cayman (KY)

(72) Inventors: Shenyi Shi, Shanghai (CN); Feng Wang, Shanghai (CN); Jingwen Liu, Mountain View, CA (US); Jian Yang, Shanghai (CN)

(73) Assignee: CVI Pharmaceuticals Limited, Grand Cayman (KY)

(21) Appl. No.: 18/115,995

(22) Filed: Mar. 1, 2023

Related U.S. Application Data
(63) Continuation of application No. PCT/CN2021/116364, filed on Sep. 3, 2021.

(30) Foreign Application Priority Data
Sep. 3, 2020 (WO) PCT/CN2020/113235

(51) Int. Cl. C07D 471/04

(52) U.S. Cl. CPC C07D 471/04; C07B 2200/13

(57) ABSTRACT

Crystalline salts of compounds, methods of making the same, and methods of treatment of dyslipidemia including administration of the crystalline salts, are provided.

### CROSS REFERENCE TO RELATED APPLICATIONS

This application is a continuation of International Patent Application No. PCT/CN2021/116364, filed Sep. 3, 2021, which claims the benefit of PCT/CN2020/113235, filed on Sep. 3, 2020, the entire disclosure of each is hereby incorporated by reference for any and all purposes.

### FIELD

This disclosure relates generally to polymorphs of crystalline forms of (R)- and (S)-enantiomers of 3,10-dimethoxy-5,8,13,13a-tetrahydro-6H-isoquinolino[3,2-a]isoquinolin-9-yl 3-fluorobenzenesulfonate, including salts. Specifically, the disclosure relates to polymorphs of the crystalline freebase and the crystalline HCl, mesylate, sulfate, hydrobromide, besylate, tosylate and maleate salts of the foregoing compounds, as well as uses thereof.

### BACKGROUND

Dyslipidemia is an abnormal amount of lipids (e.g., triglycerides, cholesterol and/or fat phospholipids) in the blood. Hyperlipidemia, refers to elevated blood lipid levels. Common forms of hyperlipidemia include hypercholesterolemia (elevated blood cholesterol) and hypertriglyceridemia (elevated blood triglycerides).

Elevated total cholesterol occurs in 13% of the population in the United States.¹ Increased levels of lipids and cholesterol can result in fatty deposits in blood vessels leading to heart disease, stroke, and death. Thus, the use of medications which can regulate lipid levels and lower the risk for these outcomes is important.

Compounds 1a and 1b, the (R)- and (S)-enantiomers respectively, of 3,10-dimethoxy-5,8,13,13a-tetrahydro-6H-isoquinolino[3,2-a]isoquinolin-9-yl 3-fluorobenzenesulfonate, have the structures:

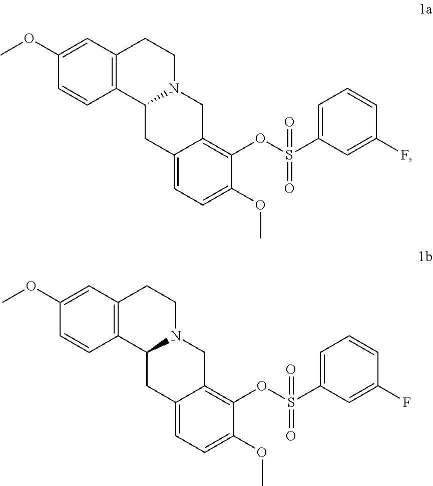 [SMILES: COc1ccc2c(c1)CCN1Cc3c(ccc(OC)c3OS(=O)(=O)c3cccc(F)c3)C[C@H]21 ; COc1ccc2c(c1)CCN1Cc3c(ccc(OC)c3OS(=O)(=O)c3cccc(F)c3)C[C@@H]21]

They are PCSK9 modulators with distinct mechanisms of action in lowering blood atherogenic LDL-cholesterol and reducing liver fat. Working in synergy with statins, aa or 1b has the potential to target patients with hypercholesterolemia and NAFLD/NASH patients with elevated LDL-C.

Compound 1a has undergone a double-blind, randomized phase 1a study conducted in healthy volunteers. Compared to baseline, serum PCSK9 levels were significantly reduced after 10 days of oral treatment with 1a (300 mg, QD). Moreover, in a proof-of-mechanism phase 1b study conducted in subjects with elevated LDL-C, compared with the placebo cohort, treatment with 1a for 28 days significantly reduced serum LDL-C, TC, Apo B, and PCSK9 levels. Compound 1a displayed a favorable safety profile and was well tolerated in phase 1 studies conducted in healthy volunteers and hyperlipidemic subjects. Currently, a phase 2 POC trial is underway in China in patients with hypercholesterolemia.

### SUMMARY

Thermal stability and/or the absence of hygroscopicity are important physical properties for pharmaceutical compounds to possess. Forming various salts and polymorphs of a pharmaceutical compound can result in differing crystal lattice configurations, leading to differences in these important physical properties across each particular polymorph or salt formed. The present technology provides stable salts of compound 1a and 1b, as well as a stable freebase polymorph. In some embodiments, the polymorphs are non-hygroscopic (i.e., absorb <0.2 wt % water).

In one aspect, the present technology provides a crystalline compound selected from the crystalline freebase or crystalline hydrochloride, hydrobromide, sulfate, mesylate, besylate, tosylate, or maleate salt of the compound of formula 1a or formula 1b,

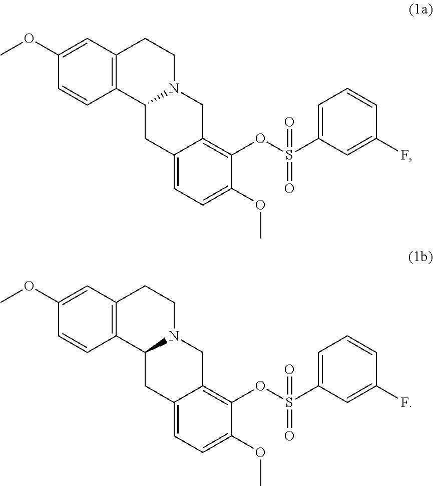 [SMILES: COc1ccc2c(c1)CCN1Cc3c(ccc(OC)c3OS(=O)(=O)c3cccc(F)c3)C[C@H]21 ; COc1ccc2c(c1)CCN1Cc3c(ccc(OC)c3OS(=O)(=O)c3cccc(F)c3)C[C@@H]21]

In any embodiment, a crystalline compound which is the HCl salt polymorph of formula I or formula II is provided:

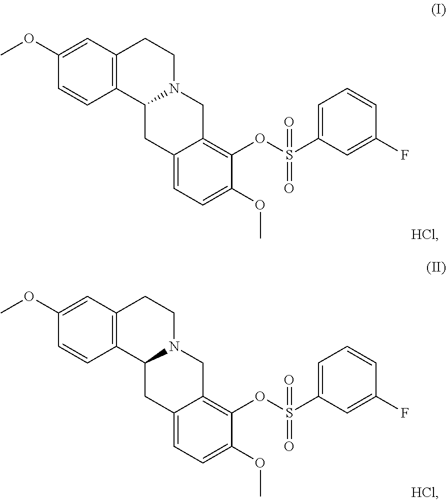 [SMILES: COc1ccc2c(c1)CCN1Cc3c(ccc(OC)c3OS(=O)(=O)c3cccc(F)c3)C[C@H]21 ; "HCl," ; COc1ccc2c(c1)CCN1Cc3c(ccc(OC)c3OS(=O)(=O)c3cccc(F)c3)C[C@@H]21 ; "HCl,"]

characterized by an X-ray powder diffraction pattern comprising the peaks, expressed in degrees 2θ, of 15.88±0.3, 25.00±0.3, and 20.38±0.3.

In any embodiment, a crystalline compound which is the mesylate salt polymorph of formula III or formula IV is provided:

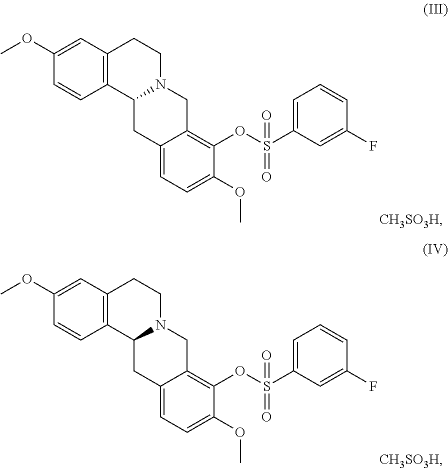 [SMILES: COc1ccc2c(c1)CCN1Cc3c(ccc(OC)c3OS(=O)(=O)c3cccc(F)c3)C[C@H]21 ; "CH3SO3H," ; COc1ccc2c(c1)CCN1Cc3c(ccc(OC)c3OS(=O)(=O)c3cccc(F)c3)C[C@@H]21 ; "CH3SO3H,"]

characterized by an X-ray powder diffraction pattern comprising the peaks, expressed in degrees 2θ, of 7.099±0.3, 19.921±0.3, and 8.501±0.3.

In any embodiment, a crystalline compound which is the sulfate salt polymorph of formula IX or formula X is provided:

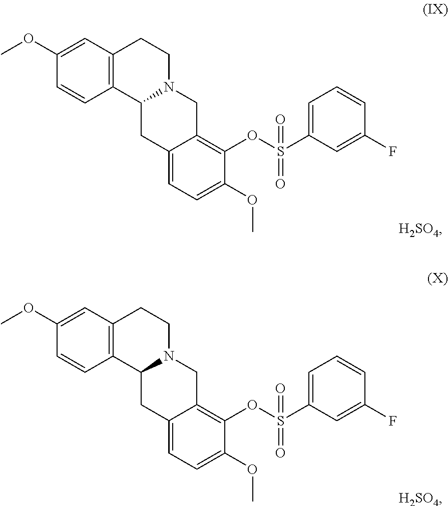 [SMILES: COc1ccc2c(c1)CCN1Cc3c(ccc(OC)c3OS(=O)(=O)c3cccc(F)c3)C[C@H]21 ; "H2SO4," ; COc1ccc2c(c1)CCN1Cc3c(ccc(OC)c3OS(=O)(=O)c3cccc(F)c3)C[C@@H]21 ; "H2SO4,"]

characterized by an X-ray powder diffraction pattern comprising the peaks, expressed in degrees 2θ, of 14.46±0.3, 22.82±0.3, 24.72±0.3.

In another aspect, a pharmaceutical composition is provided comprising a pharmaceutically effective amount of the crystalline polymorph of any embodiment herein.

In another aspect, a method for producing a crystalline polymorph of formula I or formula II is provided, comprising contacting a freebase compound of formula V or formula VI:

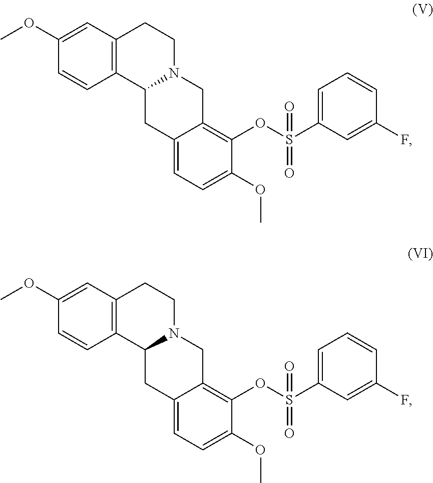 [SMILES: COc1ccc2c(c1)CCN1Cc3c(ccc(OC)c3OS(=O)(=O)c3cccc(F)c3)C[C@H]21 ; COc1ccc2c(c1)CCN1Cc3c(ccc(OC)c3OS(=O)(=O)c3cccc(F)c3)C[C@@H]21]

dissolved in a solvent, with HCl and crystallizing the crystalline hydrochloride polymorph of formula I or formula II from the solvent.

In another aspect, a method for producing a crystalline polymorph of formula III or formula IV is provided, comprising crystallizing a CH₃SO₃H salt of a freebase compound of formula V or formula VI from a solvent:

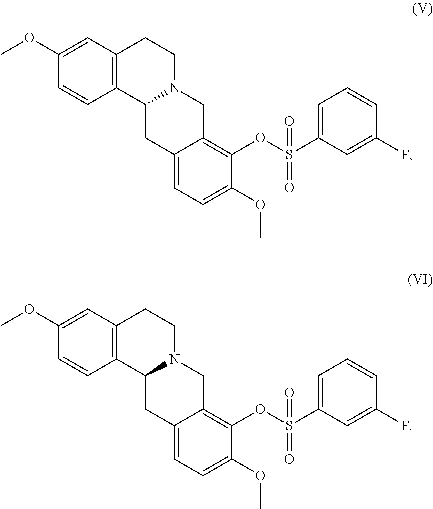 [SMILES: COc1ccc2c(c1)CCN1Cc3c(ccc(OC)c3OS(=O)(=O)c3cccc(F)c3)C[C@H]21 ; COc1ccc2c(c1)CCN1Cc3c(ccc(OC)c3OS(=O)(=O)c3cccc(F)c3)C[C@@H]21]

In another aspect, a method of treating or preventing dyslipidemia in a subject in need thereof is provided, comprising administering an effective amount of the crystalline polymorph of any embodiment herein, to the subject.

### BRIEF DESCRIPTION OF THE DRAWINGS

FIGS. 1A and 1B: X-ray powder diffraction (XRPD) spectra of two different lots of the crystalline HCl salt of formula I (2a).

FIG. 2: Differential scanning calorimetry (DSC) thermogram of the crystalline HCl salt of formula I (2a).

FIG. 3: TGA trace of the crystalline HCl salt of formula I (2a).

FIG. 4: Dynamic vapor sorption (DVS) plot of analysis conducted on crystalline HCl salt of formula I (2a).

FIG. 5: XRPD spectra of the crystalline HCl salt of formula I (2a) and the solids left over after evaporation of a solution of 2a dissolved in, from top to bottom, methanol (MeOH), ethanol (EtOH), acetonitrile (ACN), and acetone.

FIG. 6: XRPD pattern of the crystalline HCl salt of formula I (2a) (bottom) and 2a dissolved in ACN, then precipitated with heptane (top).

FIG. 7: XRPD spectrum of the crystalline HCl salt of formula I, (2a) (bottom) and 2a dissolved in MeOH, then precipitated with heptane (top) or water (center).

FIG. 8: XRPD spectra of the crystalline HCl salt of formula I (2a) after equilibration at 25° C. in acetone, EtOH, and 50% EtOH (aq) for 7 days overlaid with the spectrum of undisturbed 2a.

FIG. 9: XRPD spectra of the crystalline HCl salt of formula I (2a) equilibrated at 25° C. in MeOH, ethyl acetate (EtOAc), and ACN for 7 days overlaid over the spectrum of undisturbed 2a.

FIG. 10: XRPD spectra of the crystalline HCl salt of formula I (2a) equilibrated at 25° C. in isopropanol (IPA), water, and tetrahydrofuran (THF) for 7 days overlaid over the spectrum of undisturbed 2a.

FIG. 11: XRPD spectra of the crystalline HCl salt of formula I (2a) equilibrated at 25° C. in, from top to bottom, ACN, acetone, 50% EtOH(aq), and EtOH for 1 day overlaid over the spectrum of undisturbed 2a.

FIG. 12: XRPD spectra of the crystalline HCl salt of formula I (2a) equilibrated at 25° C. in, from top to bottom, in water, THF, MeOH, and EtOAc for 1 day overlaid over the spectrum of undisturbed 2a.

FIG. 13: XRPD spectra of the crystalline HCl salt of formula I (2a) equilibrated at 50° C. in, from top to bottom, ACN, acetone, 50% EtOH(aq), and EtOH for 1 day overlaid over the spectrum of undisturbed 2a.

FIGS. 14A and 14B: XRPD spectra of the crystalline HCl salt of formula I (2a) equilibrated at 50° C. in, from top to bottom, water, THF, and EtOAc for 1 day overlaid over the spectrum of undisturbed 2a (FIG. 14A). XRPD spectra of the crystalline HCl salt of formula I (2a) equilibrated at 55° C. in, from top to bottom, acetone and THF for 1 day overlaid over the spectrum of undisturbed 1a (FIG. 14B).

FIG. 15: XRPD spectrum of the solid crystallized from a hot saturated EtOH solution of the crystalline HCl salt of formula I (2a) (top) overlaid on the spectrum of the crystalline polymorph 2a (bottom).

FIG. 16: Chromatogram for chirally resolved free base racemate 1, showing peaks of enantiomeric freebases of formula VI (1b) and formula V (1a).

FIG. 17: XRPD pattern of freebase of formula VI, 1b.

FIGS. 18A-18C: DSC thermograms of freebase of formula VI, 1b (FIG. 18A), crystalline HCl salt of formula II, 2b (FIG. 18B) and crystalline mesylate salt of formula IV, 3b (FIG. 18C).

FIGS. 19A-19C: TGA trace of freebase of formula VI, 1b (FIG. 19A), crystalline HCl salt of formula II, 2b (FIG. 19B) and crystalline mesylate salt of formula IV, 3b (FIG. 19C).

FIGS. 20A-20C: DVS isotherm plot of freebase of formula VI, 1b (FIG. 20A), crystalline HCl salt of formula II, 2b (FIG. 20B) and crystalline mesylate salt of formula IV, 3b (FIG. 20C).

FIG. 21: XRPD spectra of freebase of formula VI, 1b and crystalline HCl salt of formula II, 2b overlaid.

FIGS. 22A-22B: XRPD spectrum of crystalline mesylate salt of formula IV, 3b (FIG. 22A); spectrum of crystalline mesylate salt, crystallized from ethyl acetate (FIG. 22B).

FIG. 23: XRPD spectra for crystalline sulfate salt polymorph of formula X.

FIG. 24: DSC thermogram of crystalline sulfate salt of formula X.

FIG. 25: TGA trace of the crystalline sulfate salt of formula X.

FIG. 26: XRPD results for the crystalline HBr salts of formula XII crystallized from acetone and THF.

FIG. 27: DSC thermogram of crystalline HBr salt of formula XII.

FIG. 28: TGA trace of the crystalline HBr salt of formula XII.

FIG. 29: XRPD results for the crystalline tosylate salt polymorph of formula XIV, from MEK/EtOAc.

FIG. 30: DSC thermogram of crystalline tosylate salt of formula XIV, from MEK/EtOAc.

FIG. 31: TGA trace of the crystalline tosylate salt of formula XIV, from MEK/EtOAc.

FIG. 32: XRPD results for the crystalline besylate salt polymorph of formula XVI, from THF/EtOAc.

FIG. 33: DSC thermogram of crystalline besylate salt of formula XVI, from THF/EtOAc.

FIG. 34: TGA trace of the crystalline besylate salt of formula XVI, from THF/EtOAc.

FIG. 35: XRPD results for the crystalline maleate salt polymorph of formula XVIII, from acetone/EtOAc.

FIG. 36: DSC thermogram of crystalline maleate salt of formula XVIII, from acetone/EtOAc.

FIG. 37: TGA trace of the crystalline maleate salt of formula XVIII, from acetone/EtOAc.

### DETAILED DESCRIPTION

Attempted salt formation from freebase 1a (see Example 1, Scheme 1) with a variety of different acids mostly resulted in failure to produce a crystalline salt as described in the Examples. However, the present inventors unexpectedly discovered particular crystalline polymorphs which do form and possess high thermal stability and/or lack hygroscopicity. In one aspect, there are provided crystalline compounds selected from the crystalline freebase or crystalline hydrochloride, hydrobromide, sulfate, mesylate, besylate, tosylate, or maleate salt of formula 1a or formula 1b,

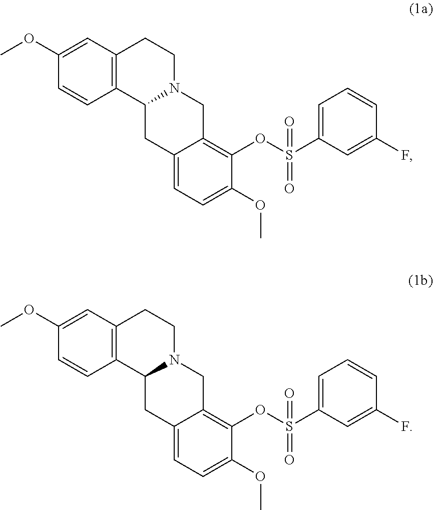 [SMILES: COc1ccc2c(c1)CCN1Cc3c(ccc(OC)c3OS(=O)(=O)c3cccc(F)c3)C[C@H]21 ; COc1ccc2c(c1)CCN1Cc3c(ccc(OC)c3OS(=O)(=O)c3cccc(F)c3)C[C@@H]21]

Polymorphs of the freebase of 1a and its enantiomer 1b, as well as crystalline salts are further described in the following embodiments.

### Polymorphs

In some embodiments, a crystalline compound which is the HCl polymorph of formula I or formula II is provided:

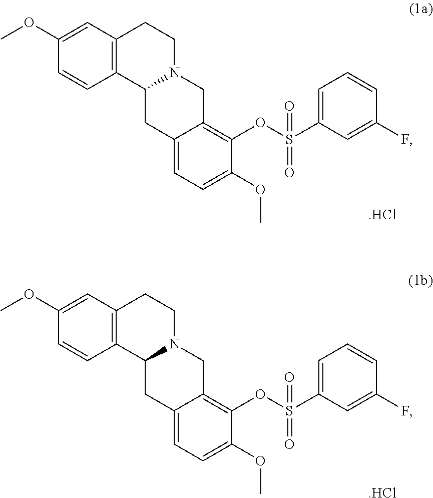 [SMILES: COc1ccc2c(c1)CCN1Cc3c(ccc(OC)c3OS(=O)(=O)c3cccc(F)c3)C[C@H]21 ; ".HCl" ; COc1ccc2c(c1)CCN1Cc3c(ccc(OC)c3OS(=O)(=O)c3cccc(F)c3)C[C@@H]21 ; ".HCl"]

characterized by an X-ray powder diffraction (XRPD) pattern comprising two or more of the peaks, expressed in degrees 2θ wherein the 2θ is ±0.3, in Table 1A:

TABLE 1A

| Peak No. | 2θ |
|---|---|
| 1 | 6.840 |
| 2 | 9.500 |
| 3 | 9.860 |
| 4 | 10.940 |
| 5 | 13.300 |
| 6 | 14.200 |
| 7 | 15.880 |
| 8 | 16.580 |
| 9 | 17.640 |
| 10 | 18.540 |
| 11 | 19.040 |
| 12 | 19.640 |
| 13 | 19.980 |
| 14 | 20.380 |
| 15 | 20.660 |
| 16 | 21.620 |
| 17 | 22.020 |
| 18 | 23.420 |
| 19 | 24.280 |
| 20 | 25.000 |
| 21 | 25.380 |
| 22 | 25.940 |
| 23 | 26.660 |
| 24 | 26.940 |
| 25 | 27.320 |
| 26 | 27.580 |
| 27 | 28.880 |
| 28 | 29.200 |
| 29 | 29.900 |
| 30 | 30.760 |
| 31 | 31.460 |
| 32 | 31.920 |
| 33 | 32.480 |
| 34 | 33.560 |
| 35 | 35.280 |
| 36 | 35.740 |
| 37 | 36.480 |
| 38 | 37.620 |
| 39 | 38.740 |
| 40 | 39.240 |
| 41 | 40.520 |
| 42 | 42.080 |
| 43 | 44.100 |
| 44 | 44.900 |
| 45 | 46.600 |

In some embodiments, the XRPD pattern comprises 2 of the peaks in Table 1A. In some embodiments, the XRPD pattern comprises 3 of the peaks in Table 1A. In some embodiments, the XRPD pattern comprises 4 of the peaks in Table 1A. In some embodiments, the XRPD pattern comprises 5 of the peaks in Table 1A. In some embodiments, the XRPD pattern comprises 6 of the peaks in Table 1A. In some embodiments, the XRPD pattern comprises 7 of the peaks in Table 1A. In some embodiments, the XRPD pattern comprises 8 of the peaks in Table 1A. In some embodiments, the XRPD pattern comprises 9 of the peaks in Table 1A. In some embodiments, the XRPD pattern comprises 10 of the peaks in Table 1A. In some embodiments, the XRPD pattern comprises 11 of the peaks in Table 1A. In some embodiments, the XRPD pattern comprises 12 of the peaks in Table 1A. In some embodiments, the XRPD pattern comprises 13 of the peaks in Table 1A. In some embodiments, the XRPD pattern comprises 14 of the peaks in Table 1A. In some embodiments, the XRPD pattern comprises 15 of the peaks in Table 1A. In some embodiments, the XRPD pattern comprises 16 of the peaks in Table 1A. In some embodiments, the XRPD pattern comprises 17 of the peaks in Table 1A. In some embodiments, the XRPD pattern comprises 18 of the peaks in Table 1A. In some embodiments, the XRPD pattern comprises 19 of the peaks in Table 1A. In some embodiments, the XRPD pattern comprises 20 of the peaks in Table 1A. In some embodiments, the XRPD pattern comprises 21 of the peaks in Table 1A. In some embodiments, the XRPD pattern comprises 22 of the peaks in Table 1A. In some embodiments, the XRPD pattern comprises 23 of the peaks in Table 1A. In some embodiments, the XRPD pattern comprises 24 of the peaks in Table 1A. In some embodiments, the XRPD pattern comprises 25 of the peaks in Table 1A. In some embodiments, the XRPD pattern comprises 26 of the peaks in Table 1A. In some embodiments, the XRPD pattern comprises 27 of the peaks in Table 1A. In some embodiments, the XRPD pattern comprises 28 of the peaks in Table 1A. In some embodiments, the XRPD pattern comprises 29 of the peaks in Table 1A. In some embodiments, the XRPD pattern comprises 30 of the peaks in Table 1A.

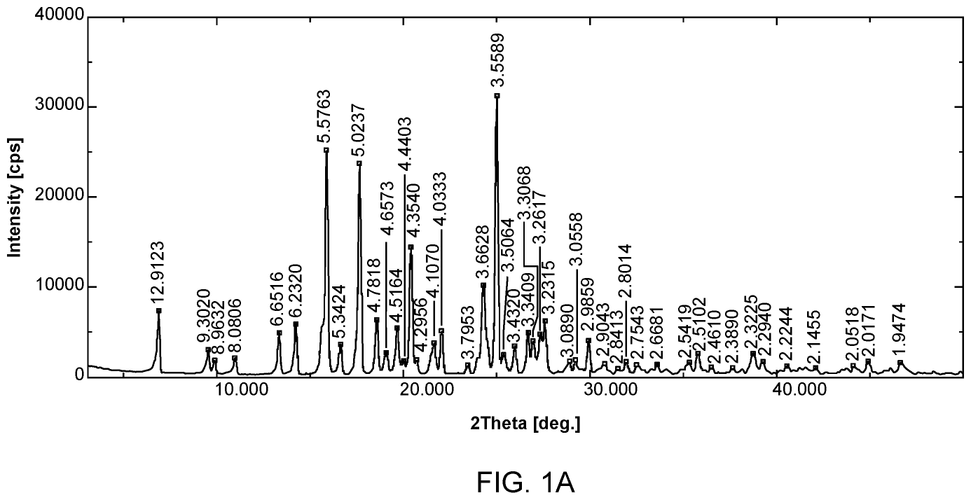

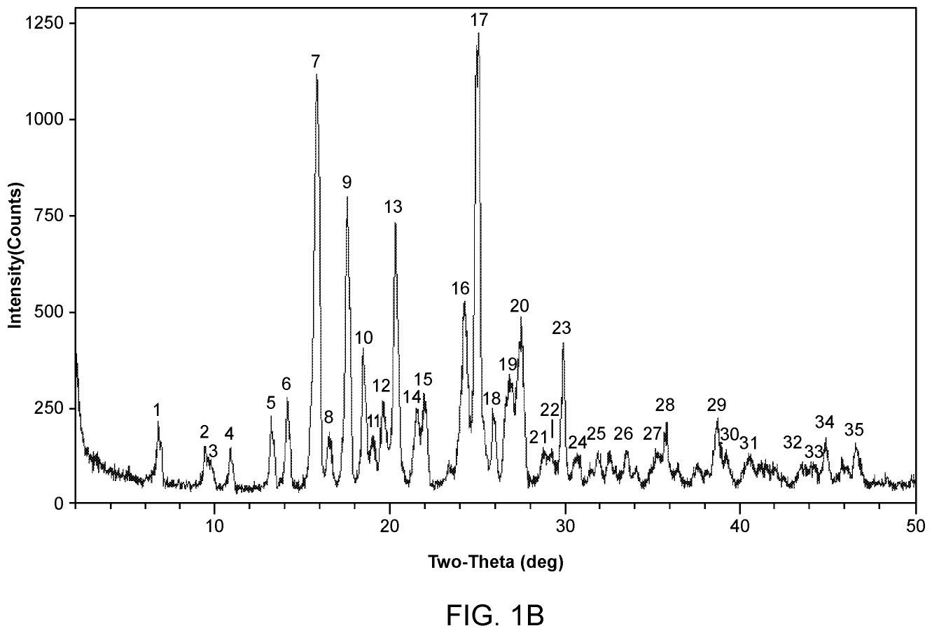

In some embodiments, the crystalline polymorph of formula I or formula II is characterized by an X-ray powder diffraction pattern comprising the peaks, expressed in degrees 2θ, of 15.88±0.3, 25.00±0.3, and 20.38±0.3. In some embodiments, the crystalline polymorph of formula I or formula II is characterized by an X-ray powder diffraction pattern further comprising the peaks, expressed in degrees 2θ, of 17.64±0.3 and 27.58±0.3. In some embodiments, the crystalline polymorph of formula I or formula II is characterized by an X-ray powder diffraction pattern further comprising the peaks, expressed in degrees 2θ, of 24.28±0.3 and 18.54±0.3. In some embodiments, the crystalline polymorph of formula I or formula II is characterized by an X-ray powder diffraction pattern further comprising the peaks, expressed in degrees 2θ, of 14.20±0.3 and 6.84±0.3. In some embodiments, the crystalline polymorph of formula I or formula II is characterized by an X-ray powder diffraction pattern further comprising the peaks, expressed in degrees 2θ, of 22.02±0.3 and 19.64±0.3. In some embodiments, the crystalline polymorph of formula I or formula II comprises the X-ray powder diffraction (XRPD) pattern substantially as shown in FIG. 1A or 1B.

In some embodiments, the crystalline polymorph of formula I or formula II is characterized by a DSC thermogram comprising an onset at about 198.4° C. (e.g., about 198° C.). In some embodiments, the onset is 198° C.±2%, and in some embodiments, the onset is 198° C.±1%. In some embodiments, the crystalline polymorph of formula I or formula II does not decompose at temperatures below 198.4° C.

In any embodiment, a crystalline compound which is the mesylate salt polymorph of formula III or formula IV is provided:

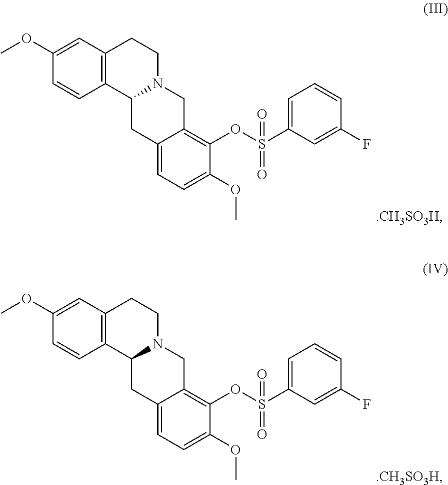 [SMILES: COc1ccc2c(c1)CCN1Cc3c(ccc(OC)c3OS(=O)(=O)c3cccc(F)c3)C[C@H]21 ; ".CH3SO3H," ; COc1ccc2c(c1)CCN1Cc3c(ccc(OC)c3OS(=O)(=O)c3cccc(F)c3)C[C@@H]21 ; ".CH3SO3H,"]

characterized by an X-ray powder diffraction pattern comprising two or more of the peaks, expressed in degrees 2θ wherein the 2θ is ±0.3, in Table 1B:

TABLE 1B

| Peak No. | 2θ |
|---|---|
| 1 | 5.979 |
| 2 | 7.099 |
| 3 | 8.501 |
| 4 | 9.482 |
| 5 | 10.120 |
| 6 | 11.558 |
| 7 | 14.142 |
| 8 | 14.601 |
| 9 | 15.839 |
| 10 | 16.917 |
| 11 | 17.679 |
| 12 | 18.139 |
| 13 | 18.843 |
| 14 | 19.255 |
| 15 | 19.921 |
| 16 | 21.076 |
| 17 | 21.641 |
| 18 | 22.483 |
| 19 | 23.059 |
| 20 | 23.520 |
| 21 | 23.900 |
| 22 | 24.979 |
| 23 | 25.404 |
| 24 | 25.839 |
| 25 | 26.322 |
| 26 | 27.563 |
| 27 | 28.441 |
| 28 | 29.719 |
| 29 | 31.601 |
| 30 | 32.099 |
| 31 | 33.161 |
| 32 | 33.522 |
| 33 | 34.077 |
| 34 | 34.983 |
| 35 | 35.361 |
| 36 | 36.677 |
| 37 | 37.263 |
| 38 | 37.842 |
| 39 | 38.220 |
| 40 | 39.284 |
| 41 | 39.700 |
| 42 | 40.219 |
| 43 | 41.720 |
| 44 | 42.720 |
| 45 | 43.801 |
| 46 | 45.676 |
| 47 | 46.720 |
| 48 | 48.716 |
| 49 | 49.357 |

In some embodiments, the crystalline polymorph of formula III or formula IV is characterized by an X-ray powder diffraction pattern comprising the peaks, expressed in degrees 2θ, of 7.099±0.3, 19.921±0.3, and 8.501±0.3. In some embodiments, the crystalline polymorph of formula III or formula IV is characterized by an X-ray powder diffraction pattern further comprising the peaks, expressed in degrees 2θ, of 16.917±0.3 and 19.255±0.3. In some embodiments, the crystalline polymorph of formula III or formula IV is characterized by an X-ray powder diffraction pattern further comprising the peaks, expressed in degrees 2θ, of 35.361±0.3 and 14.601±0.3. In some embodiments, the crystalline polymorph of formula III or formula IV is characterized by an X-ray powder diffraction pattern further comprising the peaks, expressed in degrees 2θ, of 32.099±0.3 and 28.441±0.3. In some embodiments, the crystalline polymorph of formula III or formula IV is characterized by an X-ray powder diffraction pattern further comprising the peaks, expressed in degrees 2θ, of 21.076±0.3 and 29.719±0.3.

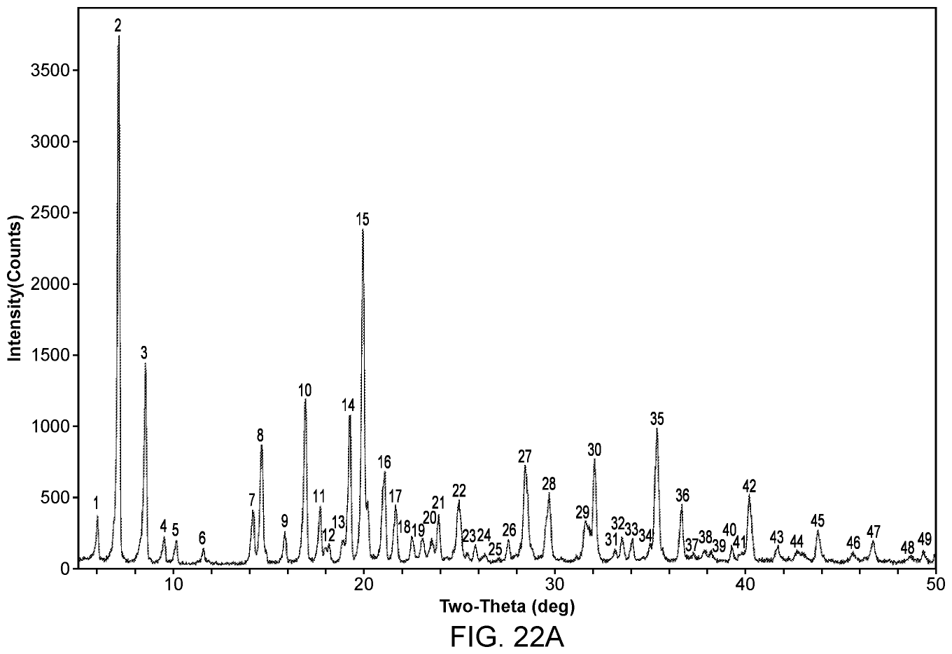

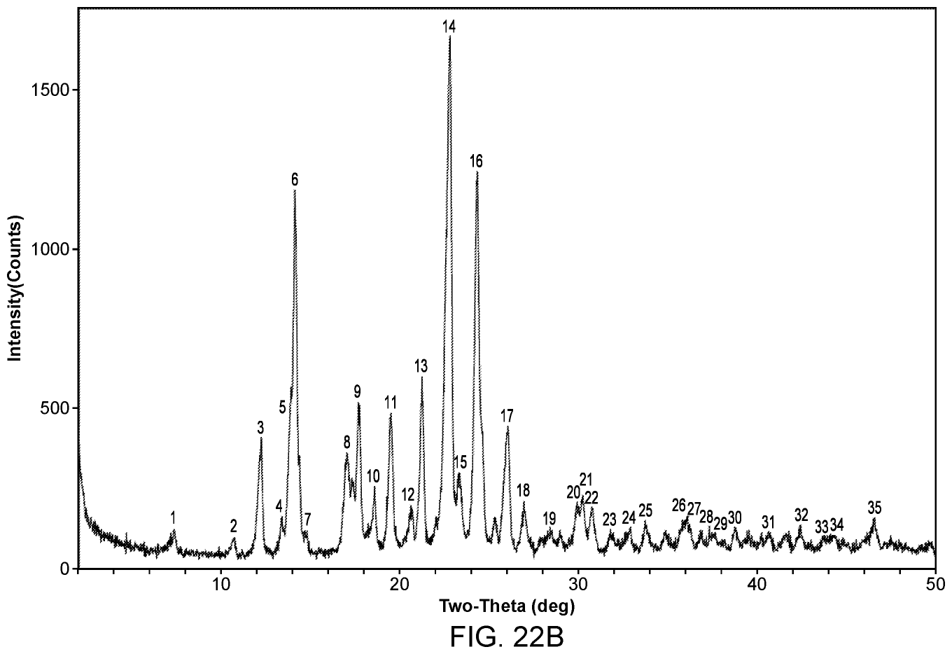

In some embodiments, the crystalline polymorph of formula III or formula IV is characterized by the X-ray powder diffraction (XRPD) pattern substantially as shown in FIG. 22.

In some embodiments, the crystalline polymorph of formula III or formula IV is characterized by a DSC thermogram comprising an onset at about 113.4° C. (e.g., about 113° C.). In some embodiments, the crystalline polymorph of formula I or formula II does not decompose at temperatures below 113.4° C.

In any embodiment, a crystalline compound which is the freebase polymorph of formula V or formula VI is provided:

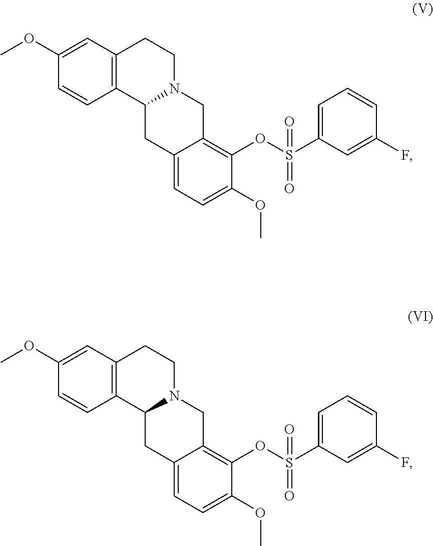 [SMILES: COc1ccc2c(c1)CCN1Cc3c(ccc(OC)c3OS(=O)(=O)c3cccc(F)c3)C[C@H]21 ; COc1ccc2c(c1)CCN1Cc3c(ccc(OC)c3OS(=O)(=O)c3cccc(F)c3)C[C@@H]21]

characterized by an X-ray powder diffraction pattern comprising two or more of the peaks, expressed in degrees 2θ wherein the 2θ is ±0.3, in Table 1C:

TABLE 1C

| Peak No. | 2θ |
|---|---|
| 1 | 9.279 |
| 2 | 10.182 |
| 3 | 11.742 |
| 4 | 12.361 |
| 5 | 16.342 |
| 6 | 17.68 |
| 7 | 18.28 |
| 8 | 19.46 |
| 9 | 20.419 |
| 10 | 22.042 |
| 11 | 23.28 |
| 12 | 24.38 |
| 13 | 25.38 |
| 14 | 26.24 |
| 15 | 26.8 |
| 16 | 27.599 |
| 17 | 29.418 |
| 18 | 29.881 |
| 19 | 30.521 |
| 20 | 32.24 |
| 21 | 33.359 |
| 22 | 34.185 |
| 23 | 34.494 |
| 24 | 34.859 |
| 25 | 36.82 |
| 26 | 38.9 |
| 27 | 40.9 |
| 28 | 41.879 |
| 29 | 42.76 |
| 30 | 45.917 |
| 31 | 48.458 |

In some embodiments of the crystalline compound which is the freebase polymorph of formula V or formula VI, the XRPD pattern comprises 2 of the peaks in Table 1C. In some embodiments, the XRPD pattern comprises 3 of the peaks in Table 1C. In some embodiments, the XRPD pattern comprises 4 of the peaks in Table 1C. In some embodiments, the XRPD pattern comprises 5 of the peaks in Table 1C. In some embodiments, the XRPD pattern comprises 6 of the peaks in Table 1C. In some embodiments, the XRPD pattern comprises 7 of the peaks in Table 1C. In some embodiments, the XRPD pattern comprises 8 of the peaks in Table 1C. In some embodiments, the XRPD pattern comprises 9 of the peaks in Table 1C. In some embodiments, the XRPD pattern comprises 10 of the peaks in Table 1C. In some embodiments, the XRPD pattern comprises 11 of the peaks in Table 1C. In some embodiments, the XRPD pattern comprises 12 of the peaks in Table 1C. In some embodiments, the XRPD pattern comprises 13 of the peaks in Table 1C. In some embodiments, the XRPD pattern comprises 14 of the peaks in Table 1C. In some embodiments, the XRPD pattern comprises 15 of the peaks in Table 1C. In some embodiments, the XRPD pattern comprises 16 of the peaks in Table 1C. In some embodiments, the XRPD pattern comprises 17 of the peaks in Table 1C. In some embodiments, the XRPD pattern comprises 18 of the peaks in Table 1C. In some embodiments, the XRPD pattern comprises 19 of the peaks in Table 1C. In some embodiments, the XRPD pattern comprises 20 of the peaks in Table 1C. In some embodiments, the XRPD pattern comprises 21 of the peaks in Table 1C. In some embodiments, the XRPD pattern comprises 22 of the peaks in Table 1C. In some embodiments, the XRPD pattern comprises 23 of the peaks in Table 1C. In some embodiments, the XRPD pattern comprises 24 of the peaks in Table 1C. In some embodiments, the XRPD pattern comprises 25 of the peaks in Table 1C. In some embodiments, the XRPD pattern comprises 26 of the peaks in Table 1C. In some embodiments, the XRPD pattern comprises 27 of the peaks in Table 1C. In some embodiments, the XRPD pattern comprises 28 of the peaks in Table 1C. In some embodiments, the XRPD pattern comprises 29 of the peaks in Table 1C. In some embodiments, the XRPD pattern comprises 30 of the peaks in Table 1C.

In some embodiments, the crystalline polymorph of formula V or formula VI is characterized by an X-ray powder diffraction pattern comprising the peaks, expressed in degrees 2θ, of characterized by an X-ray powder diffraction pattern comprising the peaks, expressed in degrees 2θ, of 18.28±0.3, 23.28±0.3, and 25.38±0.3. In some embodiments, the crystalline polymorph of formula V or formula VI is characterized by an X-ray powder diffraction pattern further comprising the peaks, expressed in degrees 2θ, of 11.742±0.3, 16.342±0.3, 26.24±0.3, and 26.8±0.3. In some embodiments, the crystalline polymorph of formula V or formula VI is characterized by an X-ray powder diffraction pattern further comprising the peaks, expressed in degrees 2θ, of 9.279±0.3, 10.182±0.3, 12.361, 20.419±0.3, and 24.38±0.3.

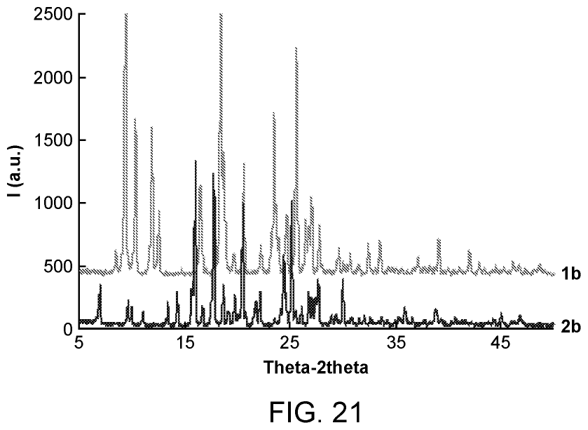

In some embodiments, the crystalline polymorph of formula V or formula VI comprises the X-ray powder diffraction (XRPD) pattern substantially as shown in FIG. 21.

In some embodiments, the crystalline polymorph of formula V or formula VI is characterized by a DSC thermogram comprising an onset at about 153.5° C. (e.g., about 154° C.). In some embodiments, the onset is 154° C.±2%, and in some embodiments, the onset is 154° C.±1%. In some embodiments, the crystalline polymorph of formula I or formula II does not decompose at temperatures below 153.5° C.

In any embodiment, a crystalline compound which is the sulfate salt polymorph of formula IX or formula X is provided:

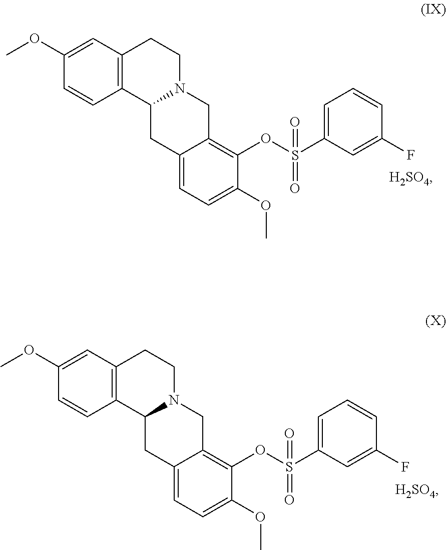

characterized by an X-ray powder diffraction pattern comprising two or more of the peaks, expressed in degrees 2θ, wherein the 2θ is ±0.3, in Table 1D:

TABLE 1D

| Peak No. | 2θ |
|---|---|
| 1 | 7.424 |
| 2 | 12.278 |
| 3 | 13.6 |
| 4 | 14.08 |
| 5 | 14.459 |
| 6 | 14.961 |
| 7 | 17.321 |
| 8 | 17.9 |
| 9 | 18.733 |
| 10 | 19.261 |
| 11 | 20.022 |
| 12 | 20.478 |
| 13 | 21.42 |
| 14 | 21.758 |
| 15 | 22.82 |
| 16 | 24.721 |
| 17 | 25.159 |
| 18 | 26.019 |
| 19 | 26.48 |
| 20 | 29.058 |
| 21 | 30.541 |
| 22 | 31.64 |
| 23 | 34.186 |
| 24 | 35.281 |
| 25 | 36.402 |
| 26 | 37.559 |
| 27 | 43.781 |
| 28 | 44.278 |
| 29 | 47.143 |

In some embodiments, the XRPD pattern comprises 3 of the peaks in Table 1D. In some embodiments, the XRPD pattern comprises 4 of the peaks in Table 1D. In some embodiments, the XRPD pattern comprises 5 of the peaks in Table 1D. In some embodiments, the XRPD pattern comprises 6 of the peaks in Table 1D. In some embodiments, the XRPD pattern comprises 7 of the peaks in Table 1D. In some embodiments, the XRPD pattern comprises 8 of the peaks in Table 1D. In some embodiments, the XRPD pattern comprises 9 of the peaks in Table 1D. In some embodiments, the XRPD pattern comprises 10 of the peaks in Table 1D. In some embodiments, the XRPD pattern comprises 11 of the peaks in Table 1D. In some embodiments, the XRPD pattern comprises 12 of the peaks in Table 1D. In some embodiments, the XRPD pattern comprises 13 of the peaks in Table 1D. In some embodiments, the XRPD pattern comprises 14 of the peaks in Table 1D. In some embodiments, the XRPD pattern comprises 15 of the peaks in Table 1D. In some embodiments, the XRPD pattern comprises 16 of the peaks in Table 1D. In some embodiments, the XRPD pattern comprises 17 of the peaks in Table 1D. In some embodiments, the XRPD pattern comprises 18 of the peaks in Table 1D. In some embodiments, the XRPD pattern comprises 19 of the peaks in Table 1D. In some embodiments, the XRPD pattern comprises 20 of the peaks in Table 1D. In some embodiments, the XRPD pattern comprises 21 of the peaks in Table 1D. In some embodiments, the XRPD pattern comprises 22 of the peaks in Table 1D. In some embodiments, the XRPD pattern comprises 23 of the peaks in Table 1D. In some embodiments, the XRPD pattern comprises 24 of the peaks in Table 1D. In some embodiments, the XRPD pattern comprises 25 of the peaks in Table 1D. In some embodiments, the XRPD pattern comprises 26 of the peaks in Table 1D. In some embodiments, the XRPD pattern comprises 27 of the peaks in Table 1D. In some embodiments, the XRPD pattern comprises 28 of the peaks in Table 1D. In some embodiments, the XRPD pattern comprises 29 of the peaks in Table 1D. In some embodiments, the XRPD pattern comprises 30 of the peaks in Table 1D.

In some embodiments, the crystalline polymorph of formula IX or X is characterized by an X-ray powder diffraction pattern comprising the peaks, expressed in degrees 2θ, of 14.46±0.3, 22.82±0.3, and 24.72±0.3. In some embodiments, the crystalline polymorph of formula V or formula VI further comprises the peaks, expressed in degrees 2θ, of 14.08±0.3, 17.32±0.3, 26.48±0.3. In some embodiments, the crystalline polymorph of formula V or formula VI further comprises the peaks, expressed in degrees 2θ, of 12.278±0.3, 17.9±0.3, 21.42±0.3, 25.159±0.3. In some embodiments, the crystalline polymorph of formula V or formula VI further comprises the peaks, expressed in degrees 2θ, of 13.6±0.3, 20.022±0.3, 20.478±0.3, 21.758±0.3.

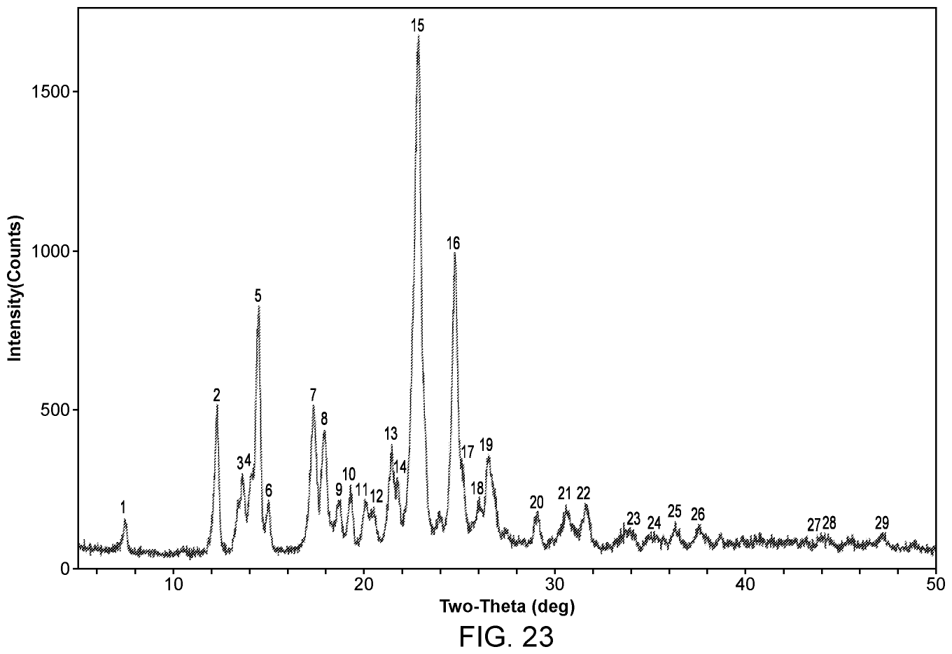

In some embodiments, the crystalline polymorph of formula IX or formula X comprises the X-ray powder diffraction (XRPD) pattern substantially as shown in FIG. 23.

In some embodiments, the crystalline polymorph of formula IX or formula X is characterized by a DSC thermogram comprising an onset at about 216.9° C. (e.g., about 217° C.). In some embodiments, the onset is 217° C.±2%, and in some embodiments, the onset is 217° C.±1%. In some embodiments, the crystalline polymorph of formula I or formula II does not decompose at temperatures below 216.9° C.

In any embodiment, a crystalline compound which is the HBr salt polymorph of formula XI or formula XII is provided:

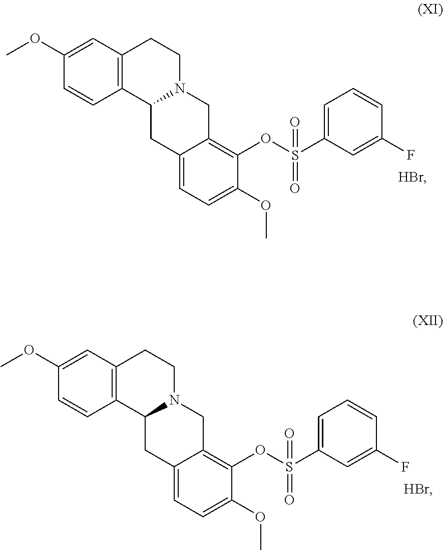

characterized by an X-ray powder diffraction pattern comprising two or more of the peaks, expressed in degrees 2θ wherein the 2θ is ±0.3, in Table 1E:

TABLE 1E

| Peak No. | 2θ |
|---|---|
| 1 | 5.416 |
| 2 | 9.706 |
| 3 | 9.981 |
| 4 | 13.496 |
| 5 | 15.555 |
| 6 | 16.339 |
| 7 | 18.614 |
| 8 | 18.913 |
| 9 | 21.471 |
| 10 | 22.982 |
| 11 | 24.5 |
| 12 | 25.735 |
| 13 | 26.381 |
| 14 | 29.172 |
| 15 | 29.637 |
| 16 | 5.416 |
| 17 | 9.706 |
| 18 | 9.981 |
| 19 | 13.496 |
| 20 | 15.555 |
| 21 | 16.339 |
| 22 | 18.614 |
| 23 | 18.913 |
| 24 | 21.471 |
| 25 | 22.982 |
| 26 | 24.5 |
| 27 | 25.735 |
| 28 | 26.381 |
| 29 | 29.172 |
| 30 | 29.637 |

In some embodiments, the XRPD pattern comprises 2 of the peaks in Table 1E. In some embodiments, the XRPD pattern comprises 3 of the peaks in Table 1E. In some embodiments, the XRPD pattern comprises 4 of the peaks in Table 1E. In some embodiments, the XRPD pattern comprises 5 of the peaks in Table 1E. In some embodiments, the XRPD pattern comprises 6 of the peaks in Table 1E. In some embodiments, the XRPD pattern comprises 7 of the peaks in Table 1E. In some embodiments, the XRPD pattern comprises 8 of the peaks in Table 1E. In some embodiments, the XRPD pattern comprises 9 of the peaks in Table 1E. In some embodiments, the XRPD pattern comprises 10 of the peaks in Table 1E. In some embodiments, the XRPD pattern comprises 11 of the peaks in Table 1E. In some embodiments, the XRPD pattern comprises 12 of the peaks in Table 1E. In some embodiments, the XRPD pattern comprises 13 of the peaks in Table 1E. In some embodiments, the XRPD pattern comprises 14 of the peaks in Table 1E. In some embodiments, the XRPD pattern comprises 15 of the peaks in Table 1E. In some embodiments, the XRPD pattern comprises 16 of the peaks in Table 1E. In some embodiments, the XRPD pattern comprises 17 of the peaks in Table 1E. In some embodiments, the XRPD pattern comprises 18 of the peaks in Table 1E. In some embodiments, the XRPD pattern comprises 19 of the peaks in Table 1E. In some embodiments, the XRPD pattern comprises 20 of the peaks in Table 1E. In some embodiments, the XRPD pattern comprises 21 of the peaks in Table 1E. In some embodiments, the XRPD pattern comprises 22 of the peaks in Table 1E. In some embodiments, the XRPD pattern comprises 23 of the peaks in Table 1E. In some embodiments, the XRPD pattern comprises 24 of the peaks in Table 1E. In some embodiments, the XRPD pattern comprises 25 of the peaks in Table 1E. In some embodiments, the XRPD pattern comprises 26 of the peaks in Table 1E. In some embodiments, the XRPD pattern comprises 27 of the peaks in Table 1E. In some embodiments, the XRPD pattern comprises 28 of the peaks in Table 1E. In some embodiments, the XRPD pattern comprises 29 of the peaks in Table 1E. In some embodiments, the XRPD pattern comprises 30 of the peaks in Table 1E.

In some embodiments, the crystalline polymorph of formula XI or formula XII is characterized by an X-ray powder diffraction pattern comprising the peaks, expressed in degrees 2θ, of 24.5±0.3, 26.381±0.3, and 18.913±0.3. In some embodiments, the crystalline polymorph of formula XI or formula XII is characterized by an X-ray powder diffraction pattern further comprising the peaks, expressed in degrees 2θ, of 16.339±0.3, 15.555±0.3, and 21.471±0.3. In some embodiments, the crystalline polymorph of formula XI or formula XII is characterized by an X-ray powder diffraction pattern further comprising the peaks, expressed in degrees 2θ, of 25.735±0.3 and 9.981±0.3. In some embodiments, the crystalline polymorph of formula XI or formula XII is characterized by an X-ray powder diffraction pattern further comprising the peaks, expressed in degrees 2θ, of 29.637±0.3 and 29.172±0.3.

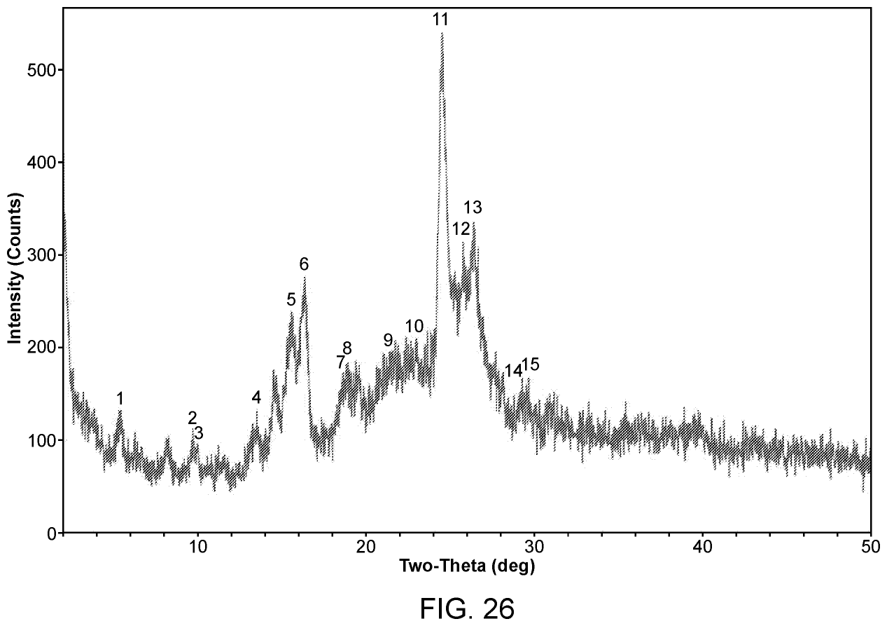

In some embodiments, the crystalline polymorph of formula XI or formula XII is characterized by the X-ray powder diffraction (XRPD) pattern substantially as shown in FIG. 26.

In some embodiments, the crystalline polymorph of formula XI or formula XII is characterized by a DSC thermogram comprising an onset at about 159.09° C. (e.g., about 159° C.). In some embodiments, the onset is 159° C.±2%, and in some embodiments, the onset is 159° C.±1%. In some embodiments, the crystalline polymorph of formula XI or formula XII does not decompose at temperatures below 120° C.

In any embodiment, a crystalline compound which is the tosylate (i.e., toluene sulfonate) salt polymorph of formula XIII or formula XIV is provided:

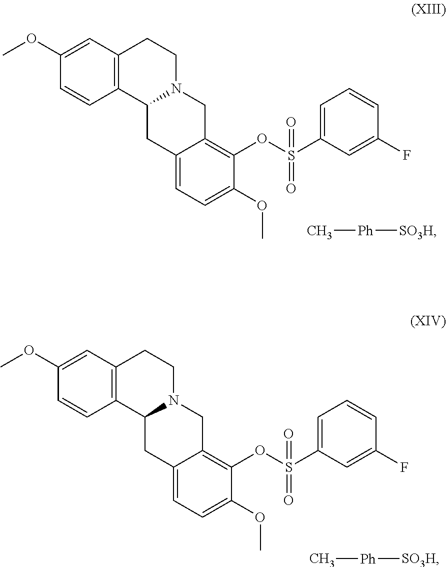

characterized by an X-ray powder diffraction pattern comprising two or more of the peaks, expressed in degrees 2θ wherein the 2θ is ±0.3, in Table 1F:

TABLE 1F

| Peak No. | 2θ |
|---|---|
| 1 | 6.381 |
| 2 | 9.241 |
| 3 | 9.859 |
| 4 | 11.883 |
| 5 | 12.547 |
| 6 | 12.863 |
| 7 | 13.362 |
| 8 | 14.241 |
| 9 | 14.843 |
| 10 | 16.56 |
| 11 | 17.401 |
| 12 | 18.661 |
| 13 | 19.463 |
| 14 | 20.059 |
| 15 | 20.281 |
| 16 | 21.14 |
| 17 | 22.521 |
| 18 | 23.139 |
| 19 | 24.141 |
| 20 | 25.4 |
| 21 | 25.8 |
| 22 | 26.078 |
| 24 | 26.999 |
| 25 | 28.28 |
| 26 | 28.944 |
| 27 | 29.379 |
| 28 | 30.199 |
| 29 | 31.221 |
| 30 | 32.516 |
| 31 | 33.12 |
| 32 | 33.659 |
| 33 | 36.037 |
| 34 | 36.499 |
| 35 | 38.437 |

In some embodiments, the XRPD pattern comprises 2 of the peaks in Table 1F. In some embodiments, the XRPD pattern comprises 3 of the peaks in Table 1F. In some embodiments, the XRPD pattern comprises 4 of the peaks in Table 1F. In some embodiments, the XRPD pattern comprises 5 of the peaks in Table 1F. In some embodiments, the XRPD pattern comprises 6 of the peaks in Table 1F. In some embodiments, the XRPD pattern comprises 7 of the peaks in Table 1F. In some embodiments, the XRPD pattern comprises 8 of the peaks in Table 1F. In some embodiments, the XRPD pattern comprises 9 of the peaks in Table 1F. In some embodiments, the XRPD pattern comprises 10 of the peaks in Table 1F. In some embodiments, the XRPD pattern comprises 11 of the peaks in Table 1F. In some embodiments, the XRPD pattern comprises 12 of the peaks in Table 1F. In some embodiments, the XRPD pattern comprises 13 of the peaks in Table 1F. In some embodiments, the XRPD pattern comprises 14 of the peaks in Table 1F. In some embodiments, the XRPD pattern comprises 15 of the peaks in Table 1F. In some embodiments, the XRPD pattern comprises 16 of the peaks in Table 1F. In some embodiments, the XRPD pattern comprises 17 of the peaks in Table 1F. In some embodiments, the XRPD pattern comprises 18 of the peaks in Table 1F. In some embodiments, the XRPD pattern comprises 19 of the peaks in Table 1F. In some embodiments, the XRPD pattern comprises 20 of the peaks in Table 1F. In some embodiments, the XRPD pattern comprises 21 of the peaks in Table 1F. In some embodiments, the XRPD pattern comprises 22 of the peaks in Table 1F. In some embodiments, the XRPD pattern comprises 23 of the peaks in Table 1F. In some embodiments, the XRPD pattern comprises 24 of the peaks in Table 1F. In some embodiments, the XRPD pattern comprises 25 of the peaks in Table 1F. In some embodiments, the XRPD pattern comprises 26 of the peaks in Table 1F. In some embodiments, the XRPD pattern comprises 27 of the peaks in Table 1F. In some embodiments, the XRPD pattern comprises 28 of the peaks in Table 1F. In some embodiments, the XRPD pattern comprises 29 of the peaks in Table 1F. In some embodiments, the XRPD pattern comprises 30 of the peaks in Table 1F.

In some embodiments, the crystalline polymorph of formula XIII or formula XIV is characterized by an X-ray powder diffraction pattern comprising the peaks, expressed in degrees 2θ, of 20.059±0.3, 24.141±0.3, and 20.281±0.3. In some embodiments, the crystalline polymorph of formula XIII or formula XIV is characterized by an X-ray powder diffraction pattern further comprising the peaks, expressed in degrees 2θ, of 21.14±0.3, 18.661±0.3, and 17.401±0.3. In some embodiments, the crystalline polymorph of formula XIII or formula XIV is characterized by an X-ray powder diffraction pattern further comprising the peaks, expressed in degrees 2θ, of 23.139±0.3 and 13.362±0.3. In some embodiments, the crystalline polymorph of formula XIII or formula XIV is characterized by an X-ray powder diffraction pattern further comprising the peaks, expressed in degrees 2θ, of 25.4±0.3 and 16.56±0.3. In some embodiments, the crystalline polymorph of formula XIII or formula XIV is characterized by an X-ray powder diffraction pattern further comprising the peaks, expressed in degrees 2θ, of 14.241±0.3.

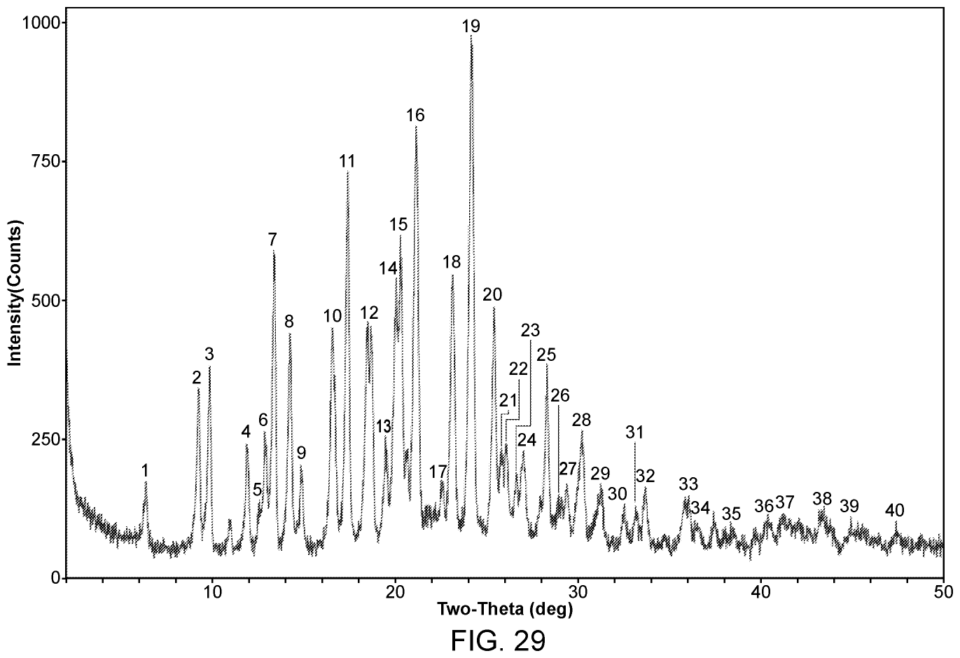

In some embodiments, the crystalline polymorph of formula XIII or formula XIV is characterized by the X-ray powder diffraction (XRPD) pattern substantially as shown in FIG. 29.

In some embodiments, the crystalline polymorph of formula XIII or formula XIV characterized by a DSC thermogram comprising an onset at about 144.9° C. (e.g., about 145° C.). In some embodiments, the onset is 145° C.±2%, and in some embodiments, the onset is 145° C.±1%. In some embodiments, the crystalline polymorph of formula XIII or formula XIV does not decompose at temperatures below 144.9° C.

In any embodiment, a crystalline compound which is the besylate (benzene sulfonate) salt polymorph of formula XV or formula XVI is provided:

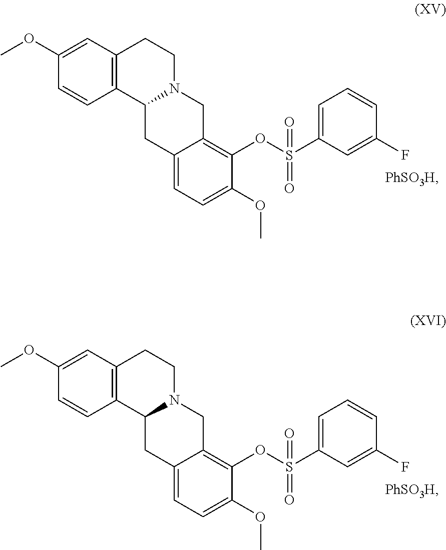

characterized by an X-ray powder diffraction pattern comprising two or more of the peaks, expressed in degrees 2θ wherein the 2θ is ±0.3, in Table 1G:

TABLE 1G

| Peak No. | 2θ |
|---|---|
| 1 | 6.638 |
| 2 | 7.758 |
| 3 | 9.638 |
| 4 | 10.319 |
| 5 | 11.86 |
| 6 | 12.538 |
| 7 | 13.759 |
| 8 | 14.457 |
| 9 | 15.56 |
| 10 | 17.24 |
| 11 | 17.842 |
| 12 | 18.38 |
| 13 | 19.264 |
| 14 | 19.562 |
| 15 | 20.799 |
| 16 | 21.921 |
| 17 | 22.2 |
| 18 | 23.599 |
| 19 | 24.4 |
| 20 | 25.079 |
| 21 | 26.238 |
| 22 | 26.957 |
| 23 | 27.602 |
| 24 | 28.94 |
| 25 | 29.287 |
| 26 | 29.745 |
| 27 | 30.094 |
| 28 | 32.018 |
| 29 | 36.579 |
| 30 | 37.197 |
| 31 | 40.867 |
| 32 | 41.216 |
| 33 | 43.177 |

In some embodiments, the XRPD pattern comprises 2 of the peaks in Table 1G. In some embodiments, the XRPD pattern comprises 3 of the peaks in Table 1G. In some embodiments, the XRPD pattern comprises 4 of the peaks in Table 1G. In some embodiments, the XRPD pattern comprises 5 of the peaks in Table 1G. In some embodiments, the XRPD pattern comprises 6 of the peaks in Table 1G. In some embodiments, the XRPD pattern comprises 7 of the peaks in Table 1G. In some embodiments, the XRPD pattern comprises 8 of the peaks in Table 1G. In some embodiments, the XRPD pattern comprises 9 of the peaks in Table 1G. In some embodiments, the XRPD pattern comprises 10 of the peaks in Table 1G. In some embodiments, the XRPD pattern comprises 11 of the peaks in Table 1G. In some embodiments, the XRPD pattern comprises 12 of the peaks in Table 1G. In some embodiments, the XRPD pattern comprises 13 of the peaks in Table 1G. In some embodiments, the XRPD pattern comprises 14 of the peaks in Table 1G. In some embodiments, the XRPD pattern comprises 15 of the peaks in Table 1G. In some embodiments, the XRPD pattern comprises 16 of the peaks in Table 1G. In some embodiments, the XRPD pattern comprises 17 of the peaks in Table 1G. In some embodiments, the XRPD pattern comprises 18 of the peaks in Table 1G. In some embodiments, the XRPD pattern comprises 19 of the peaks in Table 1G. In some embodiments, the XRPD pattern comprises 20 of the peaks in Table 1G. In some embodiments, the XRPD pattern comprises 21 of the peaks in Table 1G. In some embodiments, the XRPD pattern comprises 22 of the peaks in Table 1G. In some embodiments, the XRPD pattern comprises 23 of the peaks in Table 1G. In some embodiments, the XRPD pattern comprises 24 of the peaks in Table 1G. In some embodiments, the XRPD pattern comprises 25 of the peaks in Table 1G. In some embodiments, the XRPD pattern comprises 26 of the peaks in Table 1G. In some embodiments, the XRPD pattern comprises 27 of the peaks in Table 1G. In some embodiments, the XRPD pattern comprises 28 of the peaks in Table 1G. In some embodiments, the XRPD pattern comprises 29 of the peaks in Table 1G. In some embodiments, the XRPD pattern comprises 30 of the peaks in Table 1G.

In some embodiments, the crystalline polymorph of formula XV or formula XVI is characterized by an X-ray powder diffraction pattern comprising the peaks, expressed in degrees 2θ, of 17.842±0.3, 18.38±0.3, and 20.799±0.3. In some embodiments, the crystalline polymorph of formula XV or formula XVI is characterized by an X-ray powder diffraction pattern further comprising the peaks, expressed in degrees 2θ, of 23.599±0.3, 17.24±0.3, and 13.759±0.3. In some embodiments, the crystalline polymorph of formula XV or formula XVI is characterized by an X-ray powder diffraction pattern further comprising the peaks, expressed in degrees 2θ, of 19.562±0.3 and 22.2±0.3. In some embodiments, the crystalline polymorph of formula XV or formula XVI is characterized by an X-ray powder diffraction pattern further comprising the peaks, expressed in degrees 2θ, of 21.921±0.3 and 19.264±0.3.

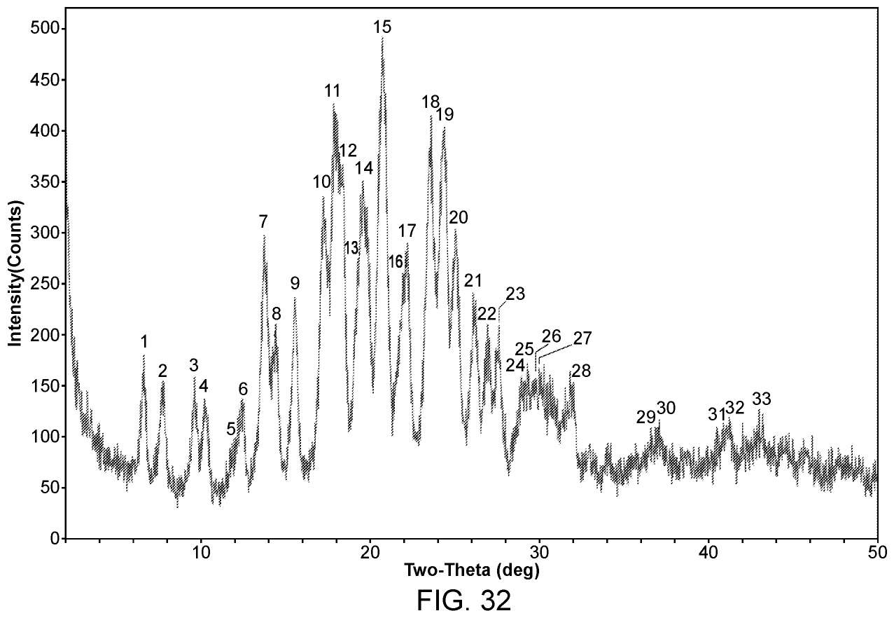

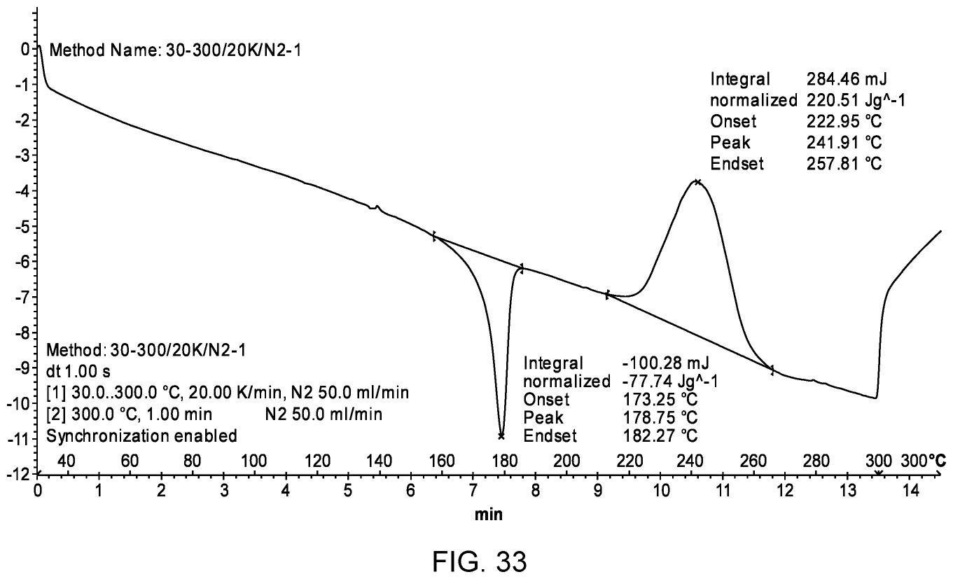

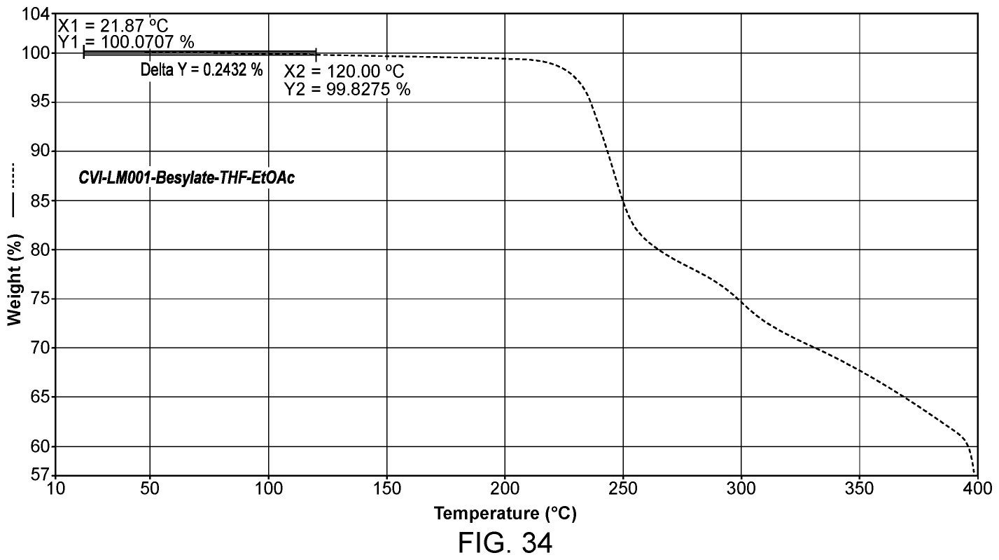

In some embodiments, the crystalline polymorph of formula XV or formula XVI is characterized by the X-ray powder diffraction (XRPD) pattern substantially as shown in FIG. 32.

In some embodiments, the crystalline polymorph of formula XV or formula XVI is characterized by a DSC thermogram comprising an onset at about 173.25° C. (e.g., about 173° C.). In some embodiments, the onset is 173° C.±2%, and in some embodiments, the onset is 173° C.±1%. In some embodiments, the crystalline polymorph of formula XV or formula XVI does not decompose at temperatures below 173.25° C.

In any embodiment, a crystalline compound which is the maleate salt polymorph of formula XVII or formula XVIII is provided:

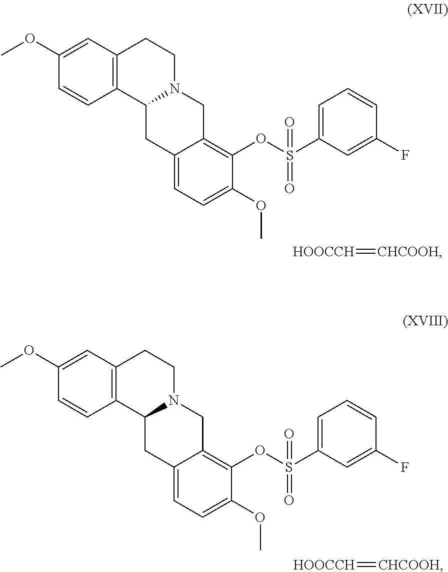

characterized by an X-ray powder diffraction pattern comprising two or more of the peaks, expressed in degrees 2θ wherein the 2θ is ±0.3, in Table 1H:

TABLE 1H

| Peak No. | 2θ |
|---|---|
| 1 | 6.779 |
| 2 | 8.437 |
| 3 | 9.861 |
| 4 | 12.058 |
| 5 | 14.578 |
| 6 | 15.7 |
| 7 | 16.36 |
| 8 | 18.121 |
| 9 | 18.8 |
| 10 | 19.276 |
| 11 | 20.259 |
| 12 | 21.361 |
| 13 | 22.68 |
| 14 | 23.302 |
| 15 | 24.237 |
| 16 | 24.9 |
| 17 | 25.259 |
| 18 | 25.964 |
| 19 | 27.12 |
| 20 | 27.461 |
| 21 | 28.86 |
| 22 | 29.319 |
| 23 | 30.217 |
| 24 | 32.383 |
| 25 | 33.14 |
| 26 | 33.8 |
| 27 | 34.758 |
| 28 | 35.061 |
| 29 | 36.157 |
| 30 | 36.637 |
| 31 | 38.02 |
| 32 | 40.384 |
| 33 | 42.105 |
| 34 | 43.36 |
| 35 | 44.204 |
| 36 | 49.332 |

In some embodiments, the XRPD pattern comprises 2 of the peaks in Table 1H. In some embodiments, the XRPD pattern comprises 3 of the peaks in Table 1H. In some embodiments, the XRPD pattern comprises 4 of the peaks in Table 1H. In some embodiments, the XRPD pattern comprises 5 of the peaks in Table 1H. In some embodiments, the XRPD pattern comprises 6 of the peaks in Table 1H. In some embodiments, the XRPD pattern comprises 7 of the peaks in Table 1H. In some embodiments, the XRPD pattern comprises 8 of the peaks in Table 1H. In some embodiments, the XRPD pattern comprises 9 of the peaks in Table 1H. In some embodiments, the XRPD pattern comprises 10 of the peaks in Table 1H. In some embodiments, the XRPD pattern comprises 11 of the peaks in Table 1H. In some embodiments, the XRPD pattern comprises 12 of the peaks in Table 1H. In some embodiments, the XRPD pattern comprises 13 of the peaks in Table 1H. In some embodiments, the XRPD pattern comprises 14 of the peaks in Table 1H. In some embodiments, the XRPD pattern comprises 15 of the peaks in Table 1H. In some embodiments, the XRPD pattern comprises 16 of the peaks in Table 1H. In some embodiments, the XRPD pattern comprises 17 of the peaks in Table 1H. In some embodiments, the XRPD pattern comprises 18 of the peaks in Table 1H. In some embodiments, the XRPD pattern comprises 19 of the peaks in Table 1H. In some embodiments, the XRPD pattern comprises 20 of the peaks in Table 1H. In some embodiments, the XRPD pattern comprises 21 of the peaks in Table 1H. In some embodiments, the XRPD pattern comprises 22 of the peaks in Table 1H. In some embodiments, the XRPD pattern comprises 23 of the peaks in Table 1H. In some embodiments, the XRPD pattern comprises 24 of the peaks in Table 1H. In some embodiments, the XRPD pattern comprises 25 of the peaks in Table 1H. In some embodiments, the XRPD pattern comprises 26 of the peaks in Table 1H. In some embodiments, the XRPD pattern comprises 27 of the peaks in Table 1H. In some embodiments, the XRPD pattern comprises 28 of the peaks in Table 1H. In some embodiments, the XRPD pattern comprises 29 of the peaks in Table 1H. In some embodiments, the XRPD pattern comprises 30 of the peaks in Table 1H.

In some embodiments, the crystalline polymorph of formula XVII or formula XVIII is characterized by an X-ray powder diffraction pattern comprising the peaks, expressed in degrees 2θ, of 23.302±0.3, 18.121±0.3, and 24.9±0.3. In some embodiments, the crystalline polymorph of formula XVII or formula XVIII is characterized by an X-ray powder diffraction pattern further comprising the peaks, expressed in degrees 2θ, of 25.259±0.3, 16.36±0.3, and 20.259±0.3. In some embodiments, the crystalline polymorph of formula XVII or formula XVIII is characterized by an X-ray powder diffraction pattern further comprising the peaks, expressed in degrees 2θ, of 21.361±0.3 and 22.68±0.3. In some embodiments, the crystalline polymorph of formula XVII or formula XVIII is characterized by an X-ray powder diffraction pattern further comprising the peaks, expressed in degrees 2θ, of 9.861±0.3 and 18.8±0.3.

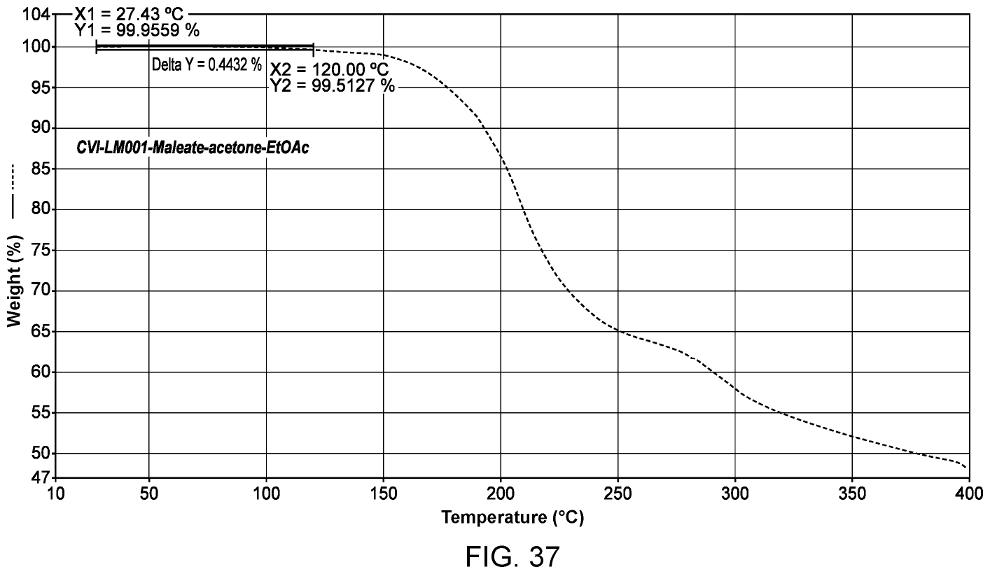

In some embodiments, the crystalline polymorph of formula XVII or formula XVIII is characterized by the X-ray powder diffraction (XRPD) pattern substantially as shown in FIG. 37.

In some embodiments, the crystalline polymorph of formula XVII or formula XVIII is characterized by a DSC thermogram comprising an onset at about 129.72° C. (e.g., about 130° C.). In some embodiments, the onset is 130° C.±2%, and in some embodiments, the onset is 130° C.±1%. In some embodiments, the crystalline polymorph of formula XVII or formula XVIII does not decompose at temperatures below 129.72° C.

In some embodiments, the crystalline polymorph of any embodiment herein comprises from about 0% to about 0.05% water by mass. In some embodiments, the crystalline polymorph of any embodiment herein comprises from about 0% to about 0.005% water by mass. In some embodiments, the crystalline polymorph of any embodiment herein comprises from about 0.005% to about 0.01% water by mass. In some embodiments, the crystalline polymorph of any embodiment herein comprises from about 0.01% to about 0.015% water by mass. In some embodiments, the crystalline polymorph of any embodiment herein comprises from about 0.015% to about 0.025% water by mass. In some embodiments, the crystalline polymorph of any embodiment herein comprises from about 0.025% to about 0.03% water by mass. In some embodiments, the crystalline polymorph of any embodiment herein comprises from about 0.03% to about 0.04% water by mass.

In some embodiments, the crystalline polymorph of any embodiment herein comprises from about 90% to about 100% enantiomeric excess (ee). In some embodiments, the crystalline polymorph of any embodiment herein comprises from about 95% to about 100% ee. In some embodiments, the crystalline polymorph of any embodiment herein comprises from about 97.5% to about 100% ee. In some embodiments, the crystalline polymorph of any embodiment herein comprises from about 99% to about 100% ee. In some embodiments, the crystalline polymorph of any embodiment herein comprises about 99% ee. In some embodiments, the crystalline polymorph of any embodiment herein comprises about 99.9% ee. In some embodiments, the crystalline polymorph of any embodiment herein comprises about 99.99% ee.

Those of skill in the art will appreciate that compounds and polymorphs of the present technology may exhibit the phenomena of tautomerism, conformational isomerism, geometric isomerism and/or stereoisomerism. As the formula drawings within the specification and claims can represent only one of the possible tautomeric, conformational isomeric, stereoisomeric or geometric isomeric forms, it should be understood that the technology encompasses any tautomeric, conformational isomeric, stereoisomeric and/or geometric isomeric forms of the compounds having one or more of the utilities described herein, as well as mixtures of these various different forms. Those having ordinary skill in the art will readily understand that two different enantiomers of the same crystalline polymorph will have the same physical properties, for example, thermal stability and hygroscopicity.

### Pharmaceutical Compositions

In another aspect, a pharmaceutical composition is provided comprising a pharmaceutically effective amount of the crystalline polymorph of any embodiment herein and a carrier. The pharmaceutical compositions of any embodiment herein may be formulated for oral, parenteral, nasal, topical administration or any of the routes discussed herein. In any embodiment herein, the pharmaceutical composition may include an effective amount of a crystalline polymorph of any embodiment of the present technology. The effective amount may be an effective amount for a dyslipidemia disclosed herein.

Such compositions may be prepared by mixing one or more polymorphs of the present technology, with pharmaceutically acceptable carriers, excipients, binders, diluents or the like to treat dyslipidemia. The polymorphs and compositions of the present technology may be used to prepare formulations and medicaments that treat a variety of dyslipidemias as disclosed herein. Such compositions can be in the form of, for example, granules, powders, tablets, capsules, creams, ointments, syrup, suppositories, injections, emulsions, inhalable compositions, aerosols, dry powders, elixirs, suspensions or solutions. The instant compositions can be formulated for various routes of administration, for example, by oral, parenteral, topical, injection, inhalation, rectal, nasal, vaginal, or via implanted reservoir. Parenteral or systemic administration includes, but is not limited to, subcutaneous, intravenous, intraperitoneally, intramuscular, intrathecal, intracranial, and intracerebroventricular injections. The following dosage forms are given by way of example and should not be construed as limiting the instant technology.

For oral, buccal, and sublingual administration, powders, suspensions, granules, tablets, pills, films, capsules, gelcaps, and caplets are acceptable as solid dosage forms. These can be prepared, for example, by mixing one or more polymorphs disclosed herein with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations (compositions) and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions, which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Polymorphs of the present technology also may be formulated as a composition for topical or transdermal administration. These formulations may contain various excipients known to those skilled in the art. Suitable excipients may include, but are not limited to, cetyl esters wax, cetyl alcohol, white wax, glyceryl monostearate, propylene glycol monostearate, methyl stearate, benzyl alcohol, sodium lauryl sulfate, glycerin, mineral oil, water, carbomer, ethyl alcohol, acrylate adhesives, polyisobutylene adhesives, and silicone adhesives.

Dosage units for rectal administration may be prepared in the form of suppositories which may contain the composition of matter in a mixture with a neutral fat base, or they may be prepared in the form of gelatin-rectal capsules which contain the active polymorph in a mixture with a vegetable oil or paraffin oil.

Polymorphs of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. Formulations for inhalation administration contain as excipients, for example, lactose, polyoxyethylene-9-lauryl ether, glycocholate and deoxycholate. Aqueous and nonaqueous aerosols are typically used for delivery of inventive polymorphs by inhalation.

Ordinarily, an aqueous aerosol is made by formulating an aqueous solution or suspension of the polymorph together with conventional pharmaceutically acceptable carriers and stabilizers. The carriers and stabilizers vary with the requirements of the particular polymorph, but typically include nonionic surfactants (Tweens, Pluronics, or polyethylene glycol), innocuous proteins such as serum albumin, sorbitan esters, oleic acid, lecithin, amino acids such as glycine, buffers, salts, sugars or sugar alcohols. Aerosols generally are prepared from isotonic solutions. A nonaqueous suspension (e.g., in a fluorocarbon propellant) can also be used to deliver polymorphs of the present technology.

Aerosols containing polymorphs for use according to the present technology are conveniently delivered using an inhaler, atomizer, pressurized pack or a nebulizer and a suitable propellant, e.g., without limitation, pressurized dichlorodifluoromethane, trichlorofluoromethane, dichlorotetrafluoroethane, nitrogen, air, or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the polymorph and a suitable powder base such as lactose or starch. Delivery of aerosols of the present technology using sonic nebulizers is advantageous because nebulizers minimize exposure of the agent to shear, which can result in degradation of the polymorph.

For nasal administration, the pharmaceutical formulations and medicaments may be a spray, nasal drops or aerosol containing an appropriate solvent(s) and optionally other compounds such as, but not limited to, stabilizers, antimicrobial agents, antioxidants, pH modifiers, surfactants, bioavailability modifiers and combinations of these. For administration in the form of nasal drops, the polymorphs may be formulated in oily solutions or as a gel. For administration of nasal aerosol, any suitable propellant may be used including compressed air, nitrogen, carbon dioxide, or a hydrocarbon based low boiling solvent.

Besides those representative dosage forms described above, pharmaceutically acceptable excipients and carriers are generally known to those skilled in the art and are thus included in the instant present technology. Such excipients and carriers are described, for example, in “Remington's Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant technology

In some embodiments, the pharmaceutical composition may comprise from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, or from about 450 mg to about 500 mg of the crystalline polymorph of any embodiment herein.

### Methods of Manufacture

In another aspect, a method for producing the crystalline polymorph of freebase formula V or formula VI (as provided herein) is provided, the method comprising dissolving the freebase compound of formula V or formula VI in a solvent, and crystallizing the crystalline polymorph from the solvent.

In another aspect, a method for producing the crystalline polymorph of formula I or formula II is provided, the method comprising contacting a freebase compound of formula V or formula VI (as provided herein) dissolved in a solvent, with HCl and crystallizing the crystalline polymorph from the solvent.

In another aspect, a method for producing the crystalline polymorph of formula XI or formula XII is provided, the method comprising contacting a freebase compound of formula V or formula VI (as provided herein) dissolved in a solvent, with HBr and crystallizing the crystalline polymorph from the solvent.

In another aspect, a method for producing the crystalline polymorph of formula IX or formula X is provided, the method comprising contacting a freebase compound of formula V or formula VI (as provided herein) dissolved in a solvent, with H₂SO₄ and crystallizing the crystalline polymorph from the solvent. In some embodiments, the H₂SO₄ is dissolved in the same solvent as the freebase compound. In some embodiments, the method comprises concentrating a solution of formula V or formula VI with H₂SO₄ or collecting a solid residue and redissolving the concentrate/solid residue in a second solvent from which the crystalline polymorph is crystallized.

In another aspect, a method for producing the crystalline polymorph of formula III or formula IV is provided, the method comprising crystallizing a salt of a freebase compound formula V or formula VI (as provided herein) with CH₃SO₃H, from a solvent. In some embodiments, the method comprises contacting the freebase compound of formula V or formula VI, dissolved in a solvent, with CH₃SO₃H. In some embodiments, the method comprises concentrating a solution of formula V or formula VI with CH₃SO₃H or collecting a solid residue and redissolving the concentrate/solid residue in a second solvent from which the crystalline polymorph is crystallized.

In another aspect, a method for producing the crystalline polymorph of formula XV or formula XVI is provided, the method comprising crystallizing a salt of a freebase compound formula V or formula VI (as provided herein) with benzenesulfonic acid, from a solvent. In some embodiments, the method comprises contacting the freebase compound of formula V or formula VI, dissolved in a solvent, with benzenesulfonic acid. In some embodiments, the method comprises concentrating a solution of formula V or formula VI with benzenesulfonic acid or collecting a solid residue and redissolving the concentrate/solid residue in a second solvent from which the crystalline polymorph is crystallized.

In another aspect, a method for producing the crystalline polymorph of formula XIII or formula XIV is provided, the method comprising crystallizing a salt of a freebase compound formula V or formula VI (as provided herein) with p-toluenesulfonic acid, from a solvent. In some embodiments, the method comprises contacting the freebase compound of formula V or formula VI, dissolved in a solvent, with p-toluenesulfonic acid. In some embodiments, the method comprises concentrating a solution of formula V or formula VI with p-toluenesulfonic acid or collecting a solid residue and redissolving the concentrate/solid residue in a second solvent from which the crystalline polymorph is crystallized.

In another aspect, a method for producing the crystalline polymorph of formula XVII or formula XVIII is provided, the method comprising crystallizing a salt of a freebase compound formula V or formula VI (as provided herein) with maleic acid, from a solvent. In some embodiments, the method comprises contacting the freebase compound of formula V or formula VI, dissolved in a solvent, with maleic acid. In some embodiments, the method comprises concentrating a solution of formula V or formula VI with maleic acid or collecting a solid residue and redissolving the concentrate/solid residue in a second solvent from which the crystalline polymorph is crystallized.

The solvent (and/or the second solvent) may comprise ethanol, methanol, propanol, isopropanol, butanol, ethyl acetate, tetrahydrofuran (THF), acetone, toluene, diethyl ether, acetonitrile, dichloromethane, methyl ethyl ketone (MEK), heptane, combinations of two or more thereof, or any organic solvent well known in the art. In some embodiments, the solvent comprises ethanol. In some embodiments, the solvent comprises methanol. In some embodiments, the solvent comprises acetone. In some embodiments, the solvent comprises ethyl acetate. In some embodiments, the solvent comprises MEK. In some embodiments, the solvent comprises THF. In some embodiments, the second solvent comprises isopropanol. In some embodiments, the second solvent comprises ethyl acetate. In some embodiments, the second solvent comprises a mixture ethyl acetate and acetone. In some embodiments, the method further comprises seeding the freebase compound in solvent with the crystalline polymorph produced by the method.

In some embodiments, the method further comprises heating the solvent with the dissolved freebase compound and the respective acid (e.g., HCl, HBr, H2504, methanesulfonic acid, benzenesulfonic acid, p-toluenesulfonic acid, or maleic acid) then cooling the solvent to crystalize the crystalline polymorph. The solvent may be heated to reflux or a to a temperature that is about 50%, about 55%, about 60%, about 65%, about 70%, about 75%, about 80%, about 85%, about 90%, about 95%, or about 100% of the boiling point of the solvent.

In some embodiments, the cooling is to a temperature that is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the boiling point of the solvent. In some embodiments, the cooling is to about 5° C. In some embodiments, the cooling is to about 0° C. In some embodiments, the cooling is to about −5° C. In some embodiments, the cooling is to about −10° C. In some embodiments, the cooling is to about 10° C. to about −10° C. In some embodiments, the cooling is to about 5° C. to about 0° C. In some embodiments, the cooling is to about −10° C. to about −30° C. In some embodiments, the cooling is to about −30° C. to about −60° C. In some embodiments, the cooling is to about −60° C. to about −90° C.

In some embodiments, the method further comprises pH adjusting to a pH greater than about 7 (e.g., about 8 to about 10 or about 8.5 to about 9.5). In some embodiments, the pH adjusting comprises adding a basic solution (e.g., basic aqueous solution including, but not limited to, NaOH solution, LiOH solution, and/or KOH) to composition comprising the compound dissolved in the solvent to crystalize the crystalline polymorph.

The freebase compound may be obtained from chiral resolution of a racemate of formula VII:

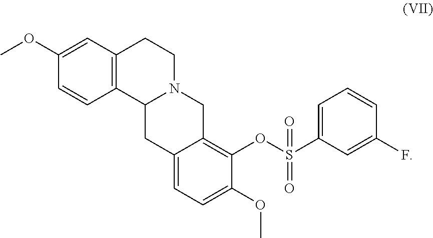 [SMILES: COc1ccc2c(c1)CCN1Cc3c(ccc(OC)c3OS(=O)(=O)c3cccc(F)c3)CC21]

In some embodiments, the chiral resolution of the racemate comprises chromatography with chiral stationary phase, contacting the racemate with chiral reagent, or entrainment and crystallization.

In some embodiments, the freebase compound obtained from chiral resolution comprises from about 90% ee to about 100% enantiomeric excess (ee). In some embodiments, the freebase compound obtained from chiral resolution comprises from about 95% ee to about 100% ee. In some embodiments, the freebase compound obtained from chiral resolution comprises from about 97.5% ee to about 100% ee. In some embodiments, the freebase compound obtained from chiral resolution comprises from about 99% ee to about 100% ee. In some embodiments, the freebase compound obtained from chiral resolution comprises about 99% ee. In some embodiments, the freebase compound obtained from chiral resolution comprises about 99.9% ee. In some embodiments, the freebase compound obtained from chiral resolution comprises about 99.99% ee. Enantiomeric excess may be ascertained by those of skill in the art by, for example, chiral liquid chromatography mass spectrometry or functionalization with a chiral auxiliary (i.e., Mosher's ester) and NMR analysis.

### Methods of Treatment

In another aspect, a method of treating or preventing dyslipidemia in a subject in need thereof is provided, comprising administering an effective amount of the crystalline polymorph of any embodiment herein, to the subject. For example, the polymorph may include the crystalline freebase or crystalline hydrochloride, hydrobromide, sulfate, mesylate, besylate, tosylate, or maleate salt of formula 1a or formula 1b as set forth herein, including but not limited to the HCl salt polymorph of formula I or formula II, the mesylate salt polymorph of formula III or formula IV, the freebase polymorph of formula V or formula VI, the sulfate salt polymorph of formula IX or formula X, the HBr salt polymorph of formula XI or formula XII, the tosylate salt polymorph of formula XIII or formula XIV, the besylate salt polymorph of formula XV or formula XVI, or the maleate salt polymorph of formula XVII or formula XVIII. In some embodiments, the dyslipidemia comprises hyperlipidemia. In some embodiments, the hyperlipidemia comprises hypercholesterolemia or hyperglyceridemia. In some embodiments, the dyslipidemia comprises hyperlipoproteinemia.

In some embodiments, the effective amount comprises from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, or from about 450 mg to about 500 mg of the crystalline polymorph of any embodiment herein.

The administration may be about once, twice, three, or four times per day, or per week. The effective amount may be delivered with each administration or between the one, two, three or four administrations per day or per week. In any embodiments, the administration may be one or two times per day or per week. In any embodiments, the administration may be once per day or per week.

Typically, the compound or compounds of the instant technology are selected to provide a formulation that exhibits a high therapeutic index. The therapeutic index is the dose ratio between toxic and therapeutic effects and can be expressed as the ratio between LD₅₀ and ED₅₀. The LD₅₀ is the dose lethal to 50% of the population and the ED₅₀ is the dose therapeutically effective in 50% of the population. The LD₅₀ and ED50 are determined by standard pharmaceutical procedures in animal cell cultures or experimental animals.

In some embodiments, the hyperlipidemia comprises elevated lipid selected from total cholesterol, triglycerides, high density lipoproteins (HDL), low density lipoproteins (LDL), or very low density lipoproteins (VLDL), in the subject. In some embodiments, the method reduces the elevated lipid in the subject by about 5%, about 10%, about 15%, about 20%, about 25%, about 30%, about 35%, about 40%, about 45%, about 50%, about 55%, about 60%, about 65%, about 70%, about 75%, or about 80%.

In some embodiments, the dyslipidemia comprises a lipid level outside of the clinical reference range for a healthy control, in the subject. In some embodiments, the method results in lipid levels in the subject within a clinically acceptable reference range. Such clinically acceptable reference ranges will be known to those of skill in the art. The healthy control may be of the same sex, age, and/or race as the subject. In some embodiments, the clinically acceptable reference range comprises <200 mg/dL for total cholesterol, <130 mg/dL for LDL, >60 mg/dL for HDL, and/or <150 mg/dL for triglycerides.

### Definitions

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively. The term “or” is inclusive unless modified, for example, by “either.” Thus, unless context or an express statement indicates otherwise, the word “or” means any one member of a particular list and also includes any combination of members of that list. Other than in the examples, or where otherwise indicated, all numbers expressing quantities of ingredients or reaction conditions used herein should be understood as modified in all instances by the term “about.”

Headings are provided for convenience only and are not to be construed to limit the invention in any way. Unless defined otherwise, all technical and scientific terms used herein have the same meaning as those commonly understood to one of ordinary skill in the art. The terminology used herein is for the purpose of describing particular embodiments only and is not intended to limit the scope of the present invention, which is defined solely by the claims. In order that the present disclosure can be more readily understood, certain terms are first defined. All numerical designations, e.g., pH, temperature, time, concentration, solubilities, and molecular weight, including ranges, are approximations which are varied (+) or (−) by increments of 1, 2, 5, or 10%. It is to be understood, although not always explicitly stated that all numerical designations are preceded by the term “about.” Numbers or quantities preceded by “about” may indicate a range of ±1%, ±2%, ±5%, or ±10% of the number to which “about” refers. It also is to be understood, although not always explicitly stated, that the reagents described herein are merely exemplary and that equivalents of such are known in the art and are set forth throughout the detailed description.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention. When an embodiment is defined by one of these terms (e.g., “comprising”) it should be understood that this disclosure also includes alternative embodiments, such as “consisting essentially of” and “consisting of” for said embodiment.

As used herein, an “effective amount” of a polymorph of the present technology refers to an amount of the polymorph by itself or in combination with another lipid lowering medicines such as a statin (HMG-CoA reductase inhibitor) that alleviates, in whole or in part, symptoms associated with a disorder or disease, or slows or halts of further progression or worsening of those symptoms, or prevents or provides prophylaxis for the disease or disorder in a subject at risk for developing the disease or disorder. Those skilled in the art are readily able to determine an effective amount. For example, one way of assessing an effective amount for a particular disease state is by simply administering a polymorph of the present technology to a patient in increasing amounts until progression of the disease state is decreased or stopped or reversed. An “effective amount” of a polymorph of the present technology also refers to an amount of the polymorph that, for example, reduces total cholesterol or reduces LDL-cholesterol or ApoB or PCSK9 in a hyperlipidemic subject to within the reference range for a healthy subject.

“Substantially” or “essentially” means nearly totally or completely, for instance, 95%, 96%, 97%, 98%, 99%, or greater of some given quantity.

The terms “subject,” “individual” or “patient” are used interchangeably herein and refer to a vertebrate, preferably a mammal. Mammals include, but are not limited to, mice, rodents, rats, simians, humans, farm animals, dogs, cats, sport animals, and pets.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the disclosure.

As used herein, the term “treatment” or “treating” means any treatment of a disease or condition or associated disorder, in a patient, including:

Inhibiting or preventing the disease or condition, that is, arresting or suppressing the development of clinical symptoms, such as neurological deficits resulting from cerebral ischemia, also included within “treatment” is provision of neuroprotection; and/or relieving the disease or condition that is, causing the regression of clinical symptoms, e.g., increasing neurological performance or reducing neurological deficits.

“Preventing” refers to stopping a healthy subject from developing a pathological condition, for example, dyslipidemia. While “treating” refers to treatment of a subject with a condition, for example, dyslipidemia.

“Compound 2a,” “formula I” or simply “2a” refers to a crystalline HCl polymorph of formula:

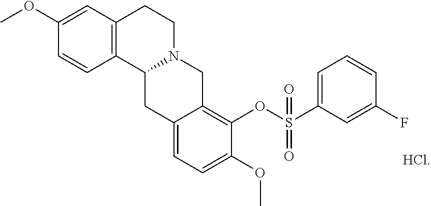 [SMILES: COc1ccc2c(c1)CCN1Cc3c(ccc(OC)c3OS(=O)(=O)c3cccc(F)c3)C[C@H]21]

Likewise, any other compounds or polymorphs referenced by a different number (i.e., 2b, 3b, 3a, etc.) correspond to their respective polymorph as defined in the following Examples.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology is further illustrated by the following examples, which should not be construed as limiting in any way.

### Examples

### Materials and Methods:

| Reagents | Grade | Manufacturer |
|---|---|---|
| Water (H₂O) | Purified | Sundia |
| Methanol (MeOH) | AR | Richjoint |
| Acetone | AR | Richjoint |
| Ethanol (EtOH) | AR | Richjoint |
| Acetonitrile (ACN) | HPLC | FULLTIME |
| Isopropanol (IPA) | AR | Richjoint |
| 2-butanone (MEK) | AR | Richjoint |
| Dichloromethane (DCM) | AR | Richjoint |
| Ethyl acetate (EtOAc) | AR | Richjoint |
| Tetrahydrofuran (THF) | HPLC | Aladdin |
| Hydrochloric acid (HCl) | AR | Richjoint |
| Hydrobromic acid (HBr) | AR | Richjoint |
| Sulfuric acid (H₂SO₄) | AR | Richjoint |
| Phosphoric acid (H₃PO₄) | HPLC | Aladdin |
| Methanesulfonic acid | CP | ACROS |
| Benzenesulfonic acid | CP | Macklin |
| P-toluenesulfonic acid | CP | Innochem |
| (TsOH) | | |
| Acetic acid (HAc) | CP | Richjoint |
| Benzoic acid | CP | Innochem |
| Fumaric acid | CP | Innochem |
| Maleic acid | CP | Innochem |
| Succinic acid | CP | Innochem |
| Citric acid | AR | Innochem |
| L-Tartaric acid | CP | Innochem |
| L-Malic acid | CP | Innochem |
| lactic acid | CP | Innochem |
| Oxalic acid | CP | Innochem |
| Formic acid | HPLC | CNW |
| Propionic acid | CP | Energy Chemical |
| Malonic acid | AR | Aladdin |
| Aspartic acid | CP | Innochem |
| L-Glutamic acid | GR | Aladdin |

XRPD (X-ray Powder Diffractometer): The X-ray powder diffraction (XRPD) pattern was obtained on a Shimadzu XRD-6000 instrument. Samples were run on XRPD using a 40 kV, 30 mA tube, Cu˜Kα (1.54056 Å) generator, and 20˜500, 5 deg/min scan scope.

DSC (Differential Scanning calorimeter): Samples of compounds (˜1 mg) were tested in a pinhole aluminum pans under nitrogen purge using a ramp rate of 20° C./min over the range 30° C.-300° C.

TGA (Thermal Gravimetric Analysis): Samples of compounds (4˜6 mg) were weighed into the pan, and heated under nitrogen purge using a ramp rate of 20° C./min over the range of 30° C.˜350° C.

DVS (Dynamic Vapor Sorption): Around 10-20 mg of samples were used to test its moisture sorption/desorption profiles at 25° C. under 0%˜95%˜0% relative humidity (RH) cycle using dm/dt: 0.01%/min equilibrium and a measurement step of 5% RH.

PLM (Polarized Light Microscope): Samples dispersed in silicone oil were observed using ocular lens: 10× and objective lens: 10× under crossed polarizers, and recorded by camera/computer system with magnification scale.

¹H-NMR (Nuclear Magnetic Resonance): About 3 mg of compound was weighed out into the nuclear magnetic resonance tube and 0.5 mL deuterated dimethyl sulfoxide or deuterated methanol was added to dissolve the sample completely. The tube was put into the rotor, and placed on the open position of the automatic sampler and scanned by BRUKER AVANCE III (400M).

Hygroscopicity Classification: Typical %-moisture gain as a function of increased RH was determined by DVS. Samples went through a dynamic test isothermally at 25° C. The test was divided into 2 processes, one was absorption process, the other was desorption process. RH ranged from 0% to 95% with 5% RH for each step, and when dm/dt<=0.01%, it was assumed the water sorption/desorption equilibrium is reached.

| Hygroscopicity Classification | Water Sorption Criterion* |
|---|---|
| Deliquescent | Sufficient water is absorbed to |
| | form a liquid |
| Very hygroscopic | W % ≥ 15% |
| Hygroscopic | W % ≥ 2% |
| Slightly hygroscopic | W % ≥ 0.2% |
| Non-hygroscopic | W % < 0.2% |

*At 25 ± 1° C. and 80 ± 2% RH (European Pharmacopoeia 6.0)

Solubility: Excess drug substance was equilibrated with SGF (Simulated Gastric Fluid), FaSSIF (Simulated Small Intestinal Fluid under Fasted state), FeSSIF (Simulated Small Intestinal Fluid under Fed state), 0.1M HCl, pH 4.5 aqueous, and pH 6.8 aqueous at 25° C. After stirring for 20˜24 h, each sample was filtered, diluted appropriately and analyzed by HPLC. The experiment was terminated once a solubility target of 5 mg/mL was reached. HPLC conditions used are shown in Table 2 below.

TABLE 2
HPLC conditions for solubility
Conditions

| Equipment | Agilent 1200 |
|---|---|
| Mobile phase | A: Water with 0.1% TFA (trifluoroacetic acid) |
| | B: ACN with 0.1% TFA |
| | A/B (v/v, 55/45) |
| Column | Eclipse XDB-C18 (5 μm, 4.6 × 150 mm) |
| | SN: USKH010297 |
| UV Detector, nm | 280 |
| Injection | 5 |
| volume, μL | |
| Column | 25 |
| temperature, ° C. | |
| Flow rate, mL/min | 1.0 |
| Run time, min | 8 |

### Example 1: Free Base Polymorph

Preparation of free base (1b): 1b was prepared by combining freebase (14.16 g, 27.8 mmol) and methanol (280.0 mL) in a 500 mL three port flask, and the compound was dissolved by stirring at room temperature. After the system was cooled to 0-5° C. with ice water, 2 mol/L NaOH aqueous solution (20.0 mL, 40.0 mmol) was slowly added (pH about 9). The reaction system was kept stirring for 1 h at 0-5° C., then filtered. The wet cake was transferred into a 100 mL single-neck flask. Purified water (52.0 mL) was added and stirred for 1 hour for cleaning excess NaOH. After filtration, the filter cake was transferred into a 100 mL single port glass bottle, and the purification procedure was repeated once. After filtration, the wet cake was moved to a watch glass, placed in a vacuum oven at 40˜45° C., and dried until constant weight. 12.4 g of white product was obtained (yield of 95%).

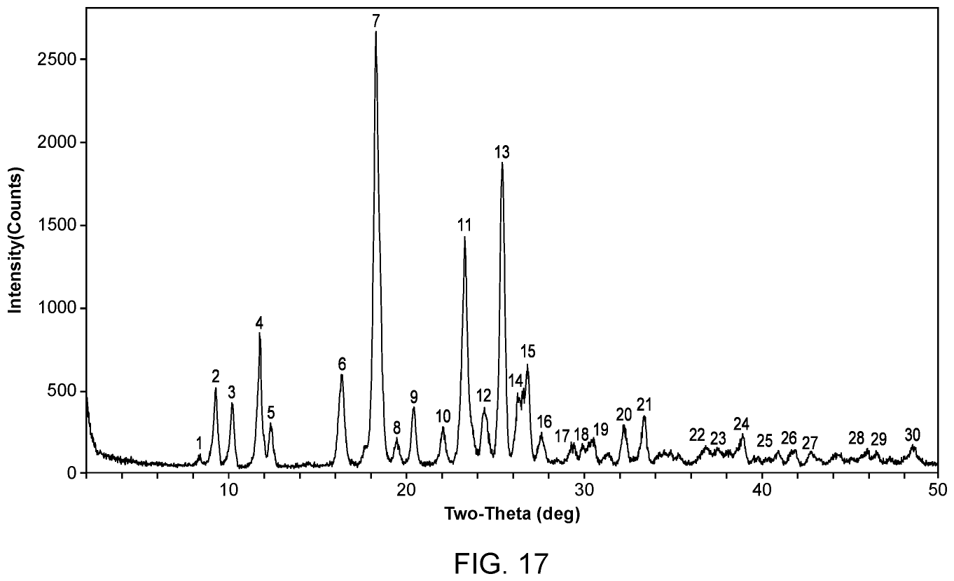

XRPD: The XRPD peak spectrum of 1b had a sharp diffraction peak and was a crystalline compound (Table 3A, FIG. 17). PLM showed birefringence and DVS shows that the compound was non-hygroscopic.

TABLE 3A
XRPD Parameters and Peaks for Crystalline Freebase of Compound 1b
Peak Search Report (31 Peaks, Max P/N = 25.2)
[Freebase(E02802-18420-09-01).RAW] CVI-LM001-Freebase
PEAK: 23-pts/Parabolic Filter, Threshold = 1.0, Cutoff = 2.0%, BG = 3/1.0,
Peak-Top = Summit

| 2-Theta | d(?) | BG | Height | I % | Area | I % | FWHM |
|---|---|---|---|---|---|---|---|
| 9.279 | 9.5229 | 65 | 453 | 17.4 | 6307 | 11.5 | 0.237 |
| 10.182 | 8.6807 | 52 | 374 | 14.3 | 5347 | 9.8 | 0.243 |
| 11.742 | 7.5302 | 47 | 803 | 30.8 | 12436 | 22.7 | 0.263 |
| 12.361 | 7.1547 | 41 | 263 | 10.1 | 4337 | 7.9 | 0.28 |
| 16.342 | 5.4198 | 55 | 543 | 20.8 | 10886 | 19.9 | 0.341 |
| 17.68 | 5.0124 | 50 | 116 | 4.4 | 1550 | 2.8 | 0.227 |
| 18.28 | 4.8492 | 67 | 2609 | 100 | 54764 | 100 | 0.357 |
| 19.46 | 4.5576 | 72 | 144 | 5.5 | 1913 | 3.5 | 0.226 |
| 20.419 | 4.3457 | 60 | 340 | 13 | 5359 | 9.8 | 0.268 |
| 22.042 | 4.0293 | 55 | 217 | 8.3 | 3427 | 6.3 | 0.268 |
| 23.28 | 3.8177 | 96 | 1336 | 51.2 | 25966 | 47.4 | 0.33 |
| 24.38 | 3.648 | 121 | 277 | 10.6 | 4884 | 8.9 | 0.3 |
| 25.38 | 3.5064 | 115 | 1767 | 67.7 | 30000 | 54.8 | 0.289 |
| 26.24 | 3.3934 | 108 | 378 | 14.5 | 14139 | 25.8 | 0.636 |
| 26.8 | 3.3237 | 98 | 562 | 21.5 | 17096 | 31.2 | 0.517 |
| 27.599 | 3.2294 | 82 | 166 | 6.4 | 2554 | 4.7 | 0.262 |
| 29.418 | 3.0337 | 86 | 98 | 3.8 | 1593 | 2.9 | 0.276 |
| 29.881 | 2.9878 | 71 | 107 | 4.1 | 4129 | 7.5 | 0.656 |
| 30.521 | 2.9265 | 77 | 139 | 5.3 | 3715 | 6.8 | 0.454 |
| 32.24 | 2.7743 | 74 | 218 | 8.4 | 3449 | 6.3 | 0.269 |
| 33.359 | 2.6837 | 80 | 268 | 10.3 | 4169 | 7.6 | 0.264 |
| 34.185 | 2.6208 | 75 | 49 | 1.9 | 1450 | 2.6 | 0.503 |
| 34.494 | 2.598 | 82 | 60 | 2.3 | 1453 | 2.7 | 0.412 |
| 34.859 | 2.5716 | 63 | 79 | 3 | 3237 | 5.9 | 0.697 |
| 36.82 | 2.439 | 83 | 85 | 3.3 | 2681 | 4.9 | 0.536 |
| 38.9 | 2.3132 | 80 | 160 | 6.1 | 2838 | 5.2 | 0.302 |
| 40.9 | 2.2046 | 67 | 71 | 2.7 | 1307 | 2.4 | 0.313 |
| 41.879 | 2.1553 | 60 | 82 | 3.1 | 1691 | 3.1 | 0.351 |
| 42.76 | 2.113 | 58 | 74 | 2.8 | 1643 | 3 | 0.377 |
| 45.917 | 1.9747 | 73 | 77 | 3 | 1635 | 3 | 0.361 |
| 48.458 | 1.877 | 59 | 113 | 4.3 | 2933 | 5.4 | 0.441 |

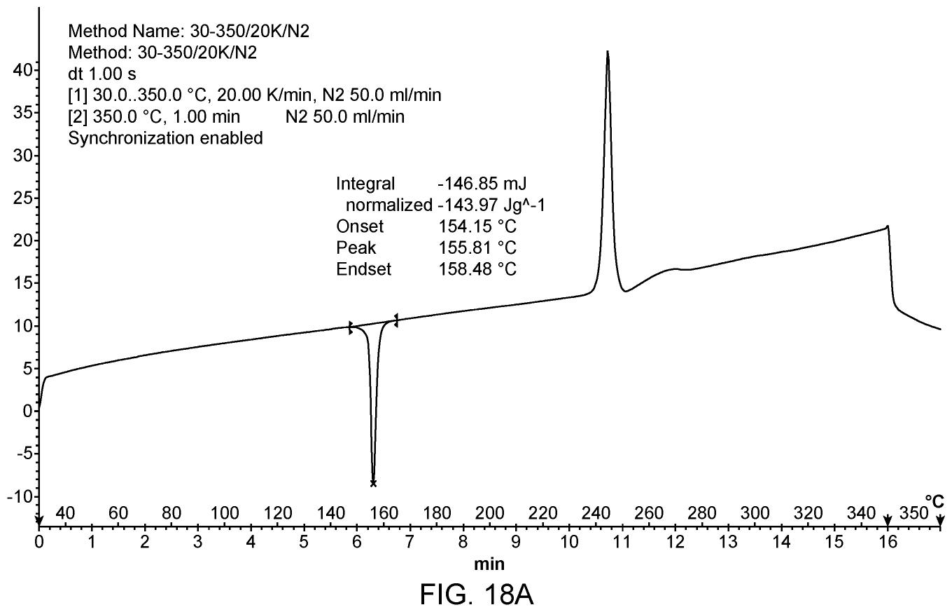

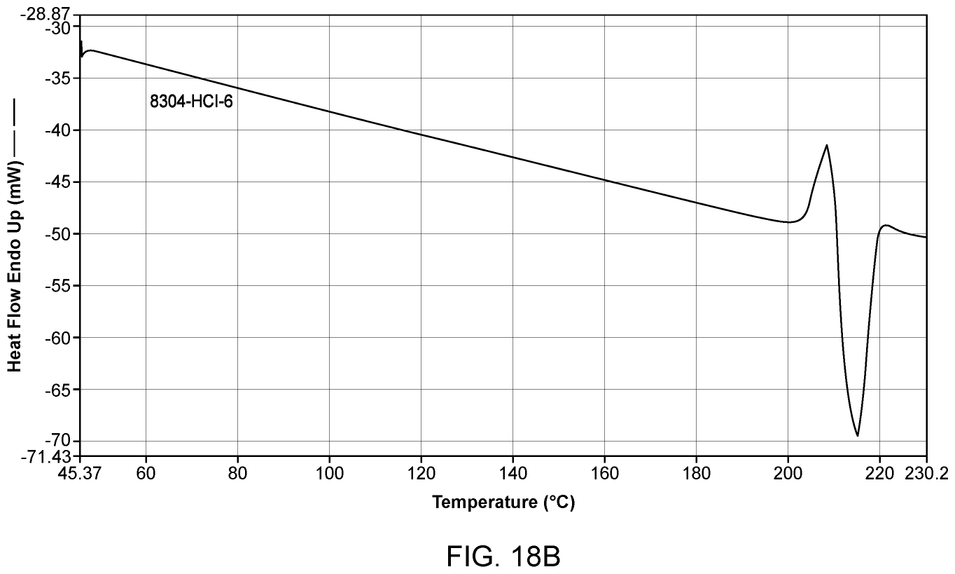

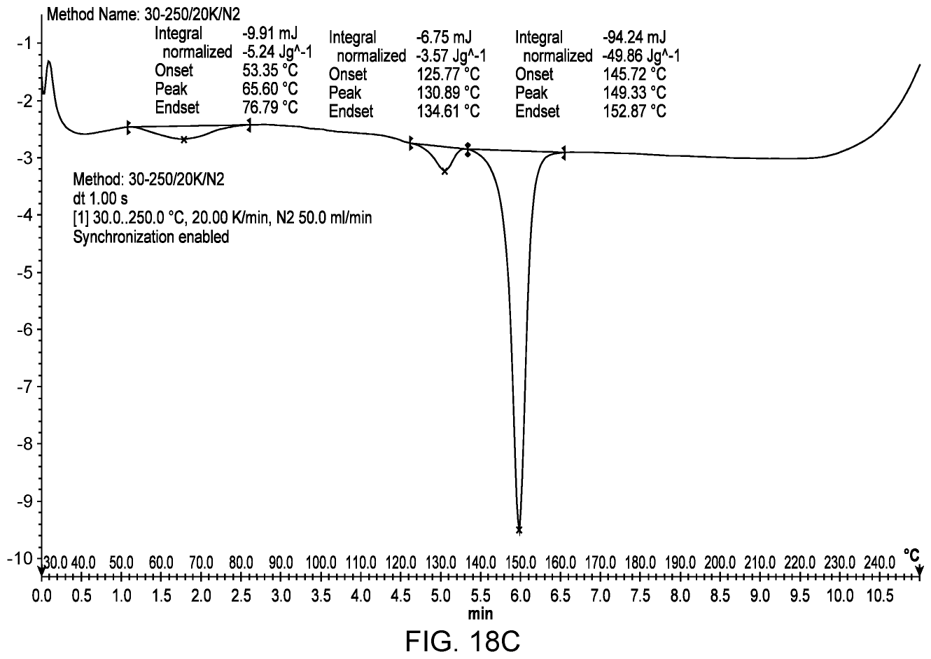

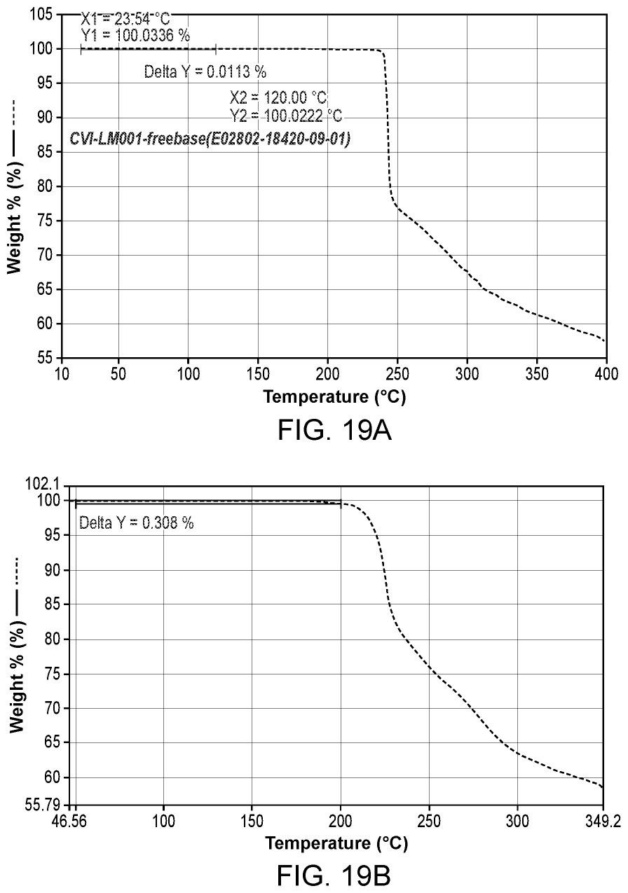

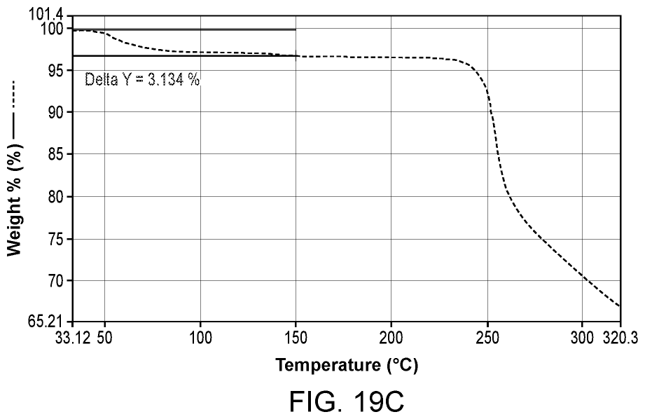

DSC and TGA: DSC thermogram showed 1b has a single melting point at about 153.45° C. (FIG. 18A). TGA showed there was almost no weight loss from room temperature to 120° C. (FIG. 19A). DSC showed an obvious endothermic peak (onset 154.15° C.) should be the melting peak of the compound.

PLM: PLM showed birefringence and DVS showed that the compound was non-hygroscopic.

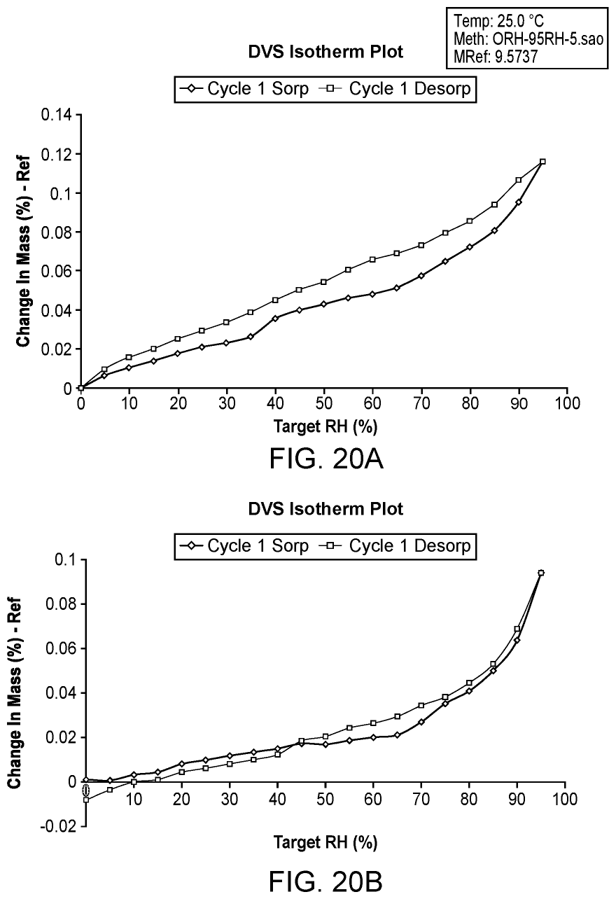

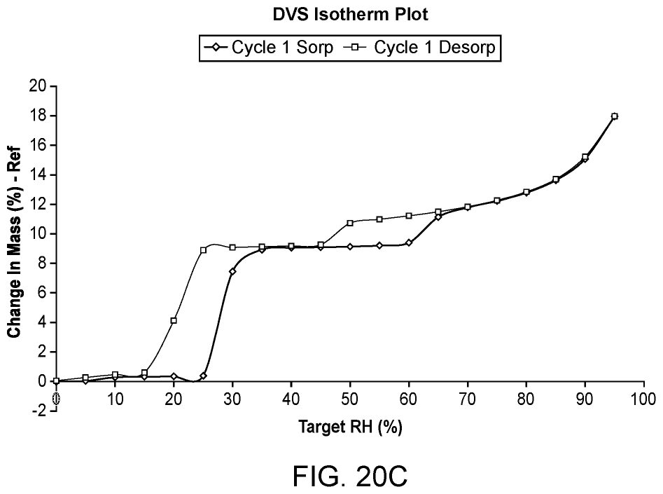

DVS Hygroscopicity of 1b: FIG. 20A and Table 3 showed that 1b was not hygroscopic, it picks up˜0.2% of moisture at 95% RH.

TABLE 3B
DVS results for 1b

| | Target | Change In Mass (%) - ref | | |
|---|---|---|---|---|
| | RH (%) | Sorption | Desorption | Hysteresis |
| Cycle 1 | 0.0 | 0.0000 | 0.0000 | |
| | 5.0 | 0.0063 | 0.0094 | 0.0031 |
| | 10.0 | 0.0104 | 0.0157 | 0.0052 |
| | 15.0 | 0.0136 | 0.0198 | 0.0063 |
| | 20.0 | 0.0178 | 0.0251 | 0.0073 |
| | 25.0 | 0.0209 | 0.0292 | 0.0084 |
| | 30.0 | 0.0230 | 0.0334 | 0.0104 |
| | 35.0 | 0.0261 | 0.0386 | 0.0125 |
| | 40.0 | 0.0355 | 0.0449 | 0.0094 |
| | 45.0 | 0.0397 | 0.0501 | 0.0104 |
| | 50.0 | 0.0428 | 0.0543 | 0.0115 |
| | 55.0 | 0.0460 | 0.0606 | 0.0146 |
| | 60.0 | 0.0480 | 0.0658 | 0.0178 |
| | 65.0 | 0.0512 | 0.0689 | 0.0178 |
| | 70.0 | 0.0574 | 0.0731 | 0.0157 |
| | 75.0 | 0.0648 | 0.0794 | 0.0146 |
| | 80.0 | 0.0721 | 0.0857 | 0.0136 |
| | 85.0 | 0.0804 | 0.0940 | 0.0136 |
| | 90.0 | 0.0951 | 0.1065 | 0.0115 |
| | 95.0 | 0.1159 | 0.1159 | |

### Example 2—Screening of Potential Salts of 1b

Various acids were tested for their ability to form a crystalline salt of freebase (1b) as follows. About 100 mg of 1b was dissolved in about 3 mL organic solvent at about 50˜60° C. and excess acid solution (1.2 eq) of each acid listed in Table 4 was added dropwise to the solution with stirring. The solid was precipitated out, filtered, and dried overnight. If the salt did not precipitate out, the solution was dried with N₂ and recrystallized from ethanol. Results are shown in Table 4.

The hydrochloride, hydrobromide, and mesylate salt polymorphs could be directly obtained by crystallization from tetrahydrofuran (THF), although among them the mesylate was obtained by solvent evaporation. Sulfate, besylate and tosylate polymorphs could be obtained by re-crystallization (slurry method) from weak polar solvents such as MTBE and ethyl acetate. Other selected acids, such as phosphoric acid, acetic acid, benzoic acid, fumaric acid, maleic acid, succinic acid, citric acid, tartaric acid, malic acid, lactic acid, oxalic acid, formic acid, propionic acid, malonic acid, aspartic acid and glutamic acid did not form crystalline polymorphs from THF.

From acetone, hydrochloride and hydrobromide polymorphs could be obtained directly. Sulfate, mesylate, benzenesulfonate, and maleate polymorphs were produced by the slurry method and recrystallization in MTBE, ethyl acetate, and isopropyl ether. Other selected acids, such as phosphoric acid, P-toluenesulfonic acid, acetic acid, fumaric acid, maleic acid, succinic acid, citric acid, L-tartaric acid, L-malic acid, lactic acid, oxalic acid, formic acid, propionic acid, malonic acid, aspartic acid, and glutamic acid, did not form crystalline polymorphs from acetone.

In THF mixed with n-heptane, where heptane was the anti-solvent, sulfate and mesylate salts could be formed and subsequently recrystallized from acetone or ethyl acetate to obtain crystalline polymorphs. Other acids such as phosphoric acid, benzenesulfonic acid, p-toluenesulfonic acid, acetic acid, benzoic acid, fumaric acid, maleic acid, succinic acid, citric acid, tartaric acid, malic acid, lactic acid, oxalic acid, formic acid, propionic acid, malonic acid, aspartic acid and glutamic acid could only be obtained as an oil or precipitated in the form of free base.

From ethyl acetate, sulfate and mesylate salt polymorphs could be obtained by recrystallization from ethyl acetate and acetone by slurry method. Other selected acids, such as phosphoric acid, acetic acid, benzoic acid, fumaric acid, maleic acid, succinic acid, citric acid, tartaric acid, maleic acid, lactic acid, oxalic acid, formic acid, propionic acid, malonic acid, aspartic acid and glutamic acid, did not form crystalline polymorphs or precipitated only as the freebase.

From methyl ethyl ketone (MEK), sulfate, mesylate, benzenesulfonate and p-toluenesulfonate salt polymorphs could be obtained by slurry method and recrystallization in petroleum ether and ethyl acetate. Other acids, such as acetic acid, benzoic acid, fumaric acid, maleic acid, succinic acid, citric acid, L-tartaric acid, L-malic acid, lactic acid, oxalic acid, formic acid, propionic acid, malonic acid, aspartic acid and glutamic acid, were mainly precipitated in the form of free base.

In order to increase the possibility of salt formation for some weak acids, a larger excess of acid was added. Two equivalents of acids were added to prepare salt in acetone. The results showed that the added phosphoric acid, acetic acid, benzoic acid, fumaric acid, maleic acid, succinic acid, citric acid, L-tartaric acid, L-malic acid, lactic acid, formic acid, propionic acid, malonic acid, aspartic acid and glutamic acid all produced an oily substance or precipitated in the form of free base.

As shown in Table 4 (below), among the 22 selected counterions, the freebase could form only seven crystalline salt polymorphs: hydrochloride salt, hydrobromide salt, sulfate, mesylate, besylate, tosylate and maleate. The remainder either did not form a salt at all or formed an amorphous salt.

TABLE 4
Salt formation of 1a with various acids

| | | | XRD (THF/n- | XRD (ethyl | |
|---|---|---|---|---|---|
| Acid | XRD (THF) | XRD (acetone) | heptane) | acetate) | XRD (MEK) |
| Hydrobromic Acid (HBr) | Crystal | Crystal | N/A | N/A | N/A |
| Hydrochloric Acid (HCl) | Crystal | Crystal | N/A | N/A | N/A |
| Phosphoric Acid (H₃PO₄) | Amorphous | Amorphous | N/A | Freebase | Amorphous |
| Citric Acid (C₆H₈O₇) | Freebase | Amorphous | N/A | Freebase | Freebase |
| L-(+)-Tartaric Acid (C₄H₆O₆) | Freebase | Freebase | Freebase | Freebase | Freebase |
| Benzenesulfonic Acid (C₆H₆O₃S) | Amorphous | Amorphous (recrystallized | N/A | N/A | (recrystallized |
| | (recrystallized in | in EtOAc-DIPE- EtOAc - | | | in EtOAc- |
| | MTBE- Amorphous; | Amorphous) | | | Crystal) |
| | Recrystallized in | | | | |
| | EtOAc-Crystal) | | | | |
| Sulfuric Acid (H₂SO₄) | Amorphous; | Amorphous; | (recrystallized in | Crystal; | (recrystallized |
| | (recrystallized in | (recrystallized in MTBE- | acetone-MEK- | (recrystallized in | in petroleum |
| | EtOAc- Crystal) | Amorphous) | Crystal) | acetone- Crystal) | ether -EtOAc- |
| | | (recrystallized in EtOAc- | | | Crystal |
| | | Crystal) | | | |
| Sulfuric Acid (½ H₂SO₄) | Amorphous; | Amorphous; | N/A | (recrystallized | Crystal; |
| | | (recrystallized in MTBE- | | in acetone- | |
| | | Freebase) | | Crystal; | |
| Acetic acid | Freebase | Freebase | Freebase | Freebase | Freebase |
| Lactic acid | (recrystallized in | Freebase | Freebase | Freebase | Freebase |
| | petroleum ether- | | | | |
| | Freebase) | | | | |
| Methanesulfonic acid | Crystal | (recrystallized in EtOAc- | (recrystallized in | (recrystallized | Crystal |
| | | Crystal) | acetone-EA-Crystal) | in acetone- | |
| | | | | EtOAc-Crystal) | |
| Maleic Acid (C₄H₄O₄) | Amorphous | Amorphous (recrystallized | Freebase | Freebase | (recrystallized |
| | (recrystallized in | in EtOAc-Crystal) | | | in EtOAc- |
| | MTBE- Amorphous) | | | | Freebase) |
| Fumaric acid (C₄H₄O₄) | Amorphous | Freebase | Freebase | Freebase | Freebase |
| | (recrystallized in | | | | |
| | MTBE-Freebase) | | | | |
| Succinic acid (C₄H₆O₄) | Freebase | Freebase | Freebase | Freebase | Freebase |
| Benzoic acid | (recrystallized in | Freebase | Freebase | Freebase | Freebase |
| | petroleum ether- | | | | |
| | Freebase) | | | | |
| Ethanesulfonic | N/A | N/A | N/A | N/A | N/A |
| L-Glutamic acid | Freebase | Freebase | Freebase | Freebase | Freebase |
| D-Glutamic acid | N/A | N/A | N/A | N/A | N/A |
| p-Toluene sulfonic acid | Amorphous | Amorphous | x | x | (recrystallized |
| | (recrystallized in | | | | in EtOAc- |
| | MTBE- Amorphous; | | | | Crystal) |
| | recrystallized in | | | | |
| | EtOAc- Crystal) | | | | |
| Malic acid | Amorphous | Freebase | Freebase | Freebase | (recrystallized |
| | | | | | in petroleum |
| | | | | | ether -EtOAc- |
| | | | | | Freebase) |
| Oxalic acid | Amorphous | Amorphous | (recrystallized in | Freebase | (recrystallized |
| | (recrystallized in | | acetone- MEK | | in petroleum |
| | MTBE- Amorphous) | | Freebase) | | ether - |
| | | | | | cyclohexane- |
| | | | | | DCM- |
| | | | | | Freebase) |
| Formic acid | Freebase | Freebase | Freebase | Freebase | Freebase |
| Propionic acid | Freebase | Freebase | Freebase | Freebase | Freebase |
| Malonic acid | Amorphous | Freebase | (recrystallized in | Freebase | (recrystallized |
| | (recrystallized in | | acetone -Freebase) | | in petroleum |
| | MTBE- Freebase) | | | | ether - |
| | | | | | Freebase) |
| Aspartic acid | Freebase | Freebase | Freebase | Freebase | Freebase |

Notes:
N/A—no results available
Amorphous—salt may have formed, but not crystalline
Crystal—salt formed crystalline material with listed acid
Freebase—salt did not form with listed acid

### Example 3: Hydrochloric acid Polymorph 2a

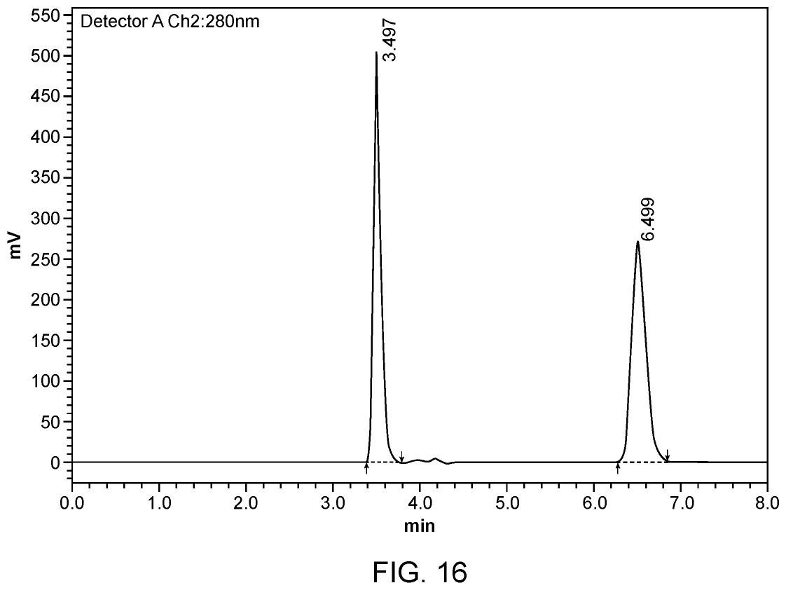

Preparation of crystalline HCl polymorph 2a: The racemate 1 may be prepared, e.g., according to the procedure in U.S. Pat. No. 8,710,071 (which yields an amorphous material). Racemate 1 was resolved into enantiomers 1b and 1a as follows. 650 g of 1a was obtained from 1350 g of 1 after chiral separation. Chiral separation was performed via HPLC with UV detection according to the parameters in Table 5 below. The two enantiomers resolved at retention times of 3.497 min and 6.499 min as shown in FIG. 16.

As shown in Scheme 1, 2a was prepared as follows: to a suspension of the free base 1a (650 g) in EtOH (2200 mL) and 95% EtOH (2600 mL) was added conc. HCl (120 mL) and 32 g of activated carbon was added at rt, then it was heated to reflux for 2 h. The solution was felted to remove the carbon. The solution was refluxed and then cooled to 60° C., the solid precipitated out, then it was cooled to 0˜5° C. The mixture was filtered and dried to furnish 608 g 2a as white-off solid.

TABLE 5
Chiral resolution parameters

| | |
|---|---|
| Column: | CHIRALPAK ICs |
| Column size: | 0.46 cm I.D. × 15 cm L |
| Injection: | 1 μl |
| Mobile phase: | Dichloromethane (DCM)/EtOAc/Diethylamine |
| | (DEA) = 50/50/0.1 |
| Flow rate: | 1.0 ml/min |
| Wave length: | UV 280 nm |
| Temperature: | 35° C. |
| Grade of solvents: | DCM, EA: HPLC grade |
| Sample structure: | Racemate |

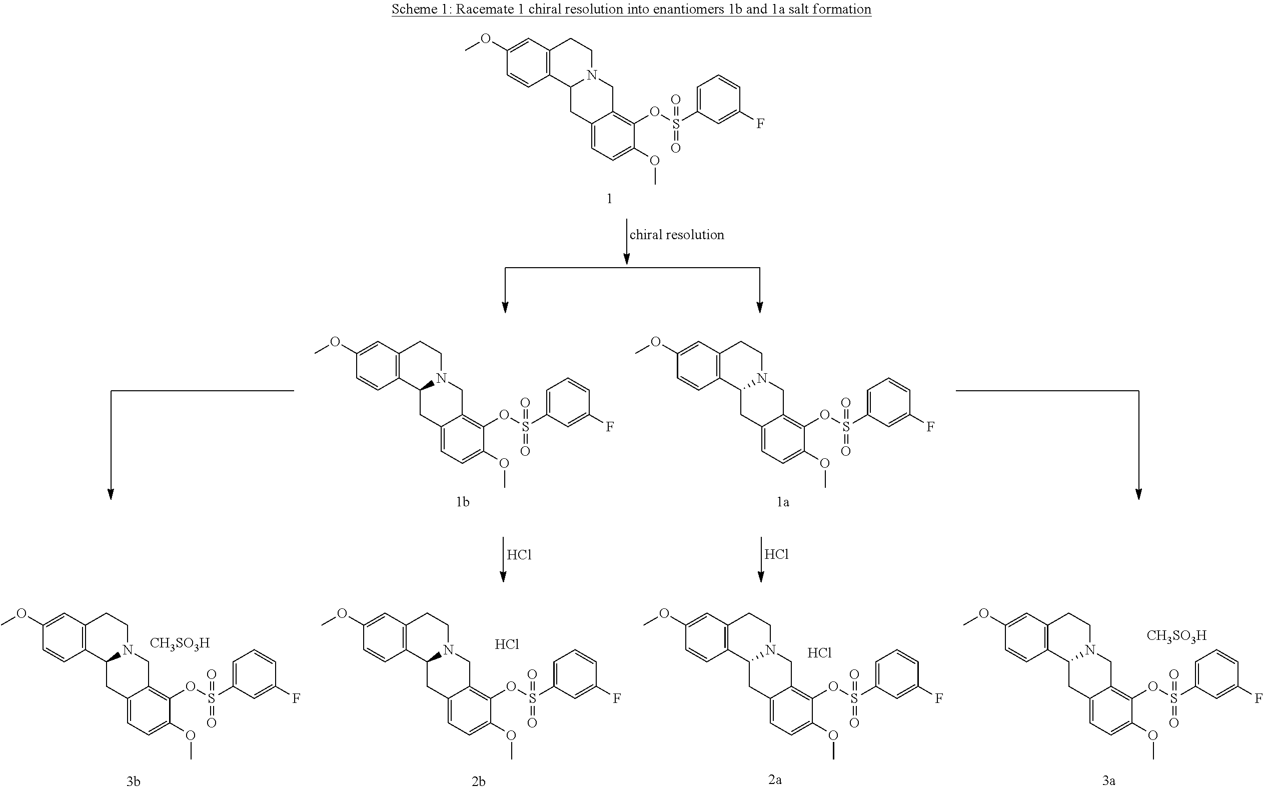

### X-Ray Powder Diffraction of 2a

2a was characterized by XRPD, TGA, DSC, and PLM. The XRPD spectra obtained for two different batches of the crystalline HCl salt of formula I are shown in FIGS. 1A and 1B. The peaks are listed in Tables 6A and 6B, respectively.

TABLE 6A
Peak table for XRPD spectrum in FIG.
1A of crystalline HCl salt 2a.

| Peak No. | 2Theta | FWHM | d-value | Intensity | I/Io |
|---|---|---|---|---|---|
| 1 | 6.840 | 0.141 | 12.9123 | 7362 | 24 |
| 2 | 9.500 | 0.165 | 9.3020 | 3012 | 10 |
| 3 | 9.860 | 0.188 | 8.9632 | 1926 | 6 |
| 4 | 10.940 | 0.165 | 8.0806 | 2162 | 7 |
| 5 | 13.300 | 0.165 | 6.6516 | 4953 | 16 |
| 6 | 14.200 | 0.188 | 6.2320 | 5900 | 19 |
| 7 | 15.880 | 0.188 | 5.5763 | 25176 | 80 |
| 8 | 16.580 | 0.165 | 5.3424 | 3673 | 12 |
| 9 | 17.640 | 0.165 | 5.0237 | 23696 | 76 |
| 10 | 18.540 | 0.188 | 4.7818 | 6371 | 20 |
| 11 | 19.040 | 0.306 | 4.6573 | 2742 | 9 |
| 12 | 19.640 | 0.188 | 4.5164 | 5469 | 17 |
| 13 | 19.980 | 0.141 | 4.4403 | 1807 | 6 |
| 14 | 20.380 | 0.188 | 4.3540 | 14530 | 46 |
| 15 | 20.660 | 0.141 | 4.2956 | 1922 | 6 |
| 16 | 21.620 | 0.188 | 4.1070 | 3794 | 12 |
| 17 | 22.020 | 0.165 | 4.0333 | 5134 | 16 |
| 18 | 23.420 | 0.235 | 3.7953 | 1268 | 4 |
| 19 | 24.280 | 0.188 | 3.6628 | 10252 | 33 |
| 20 | 25.000 | 0.188 | 3.5589 | 31291 | 100 |
| 21 | 25.380 | 0.235 | 3.5064 | 2551 | 8 |
| 22 | 25.940 | 0.188 | 3.4320 | 3432 | 11 |
| 23 | 26.660 | 0.165 | 3.3409 | 5116 | 16 |
| 24 | 26.940 | 0.165 | 3.3068 | 4018 | 13 |
| 25 | 27.320 | 0.188 | 3.2617 | 4767 | 15 |
| 26 | 27.580 | 0.165 | 3.2315 | 6215 | 20 |
| 27 | 28.880 | 0.212 | 3.0890 | 1791 | 6 |
| 28 | 29.200 | 0.165 | 3.0558 | 1941 | 6 |
| 29 | 29.900 | 0.188 | 2.9859 | 4104 | 13 |
| 30 | 30.760 | 0.188 | 2.9043 | 1522 | 5 |
| 31 | 31.460 | 0.259 | 2.8413 | 1044 | 3 |
| 32 | 31.920 | 0.212 | 2.8014 | 1777 | 6 |
| 33 | 32.480 | 0.188 | 2.7543 | 1409 | 5 |
| 34 | 33.560 | 0.188 | 2.6681 | 1473 | 5 |
| 35 | 35.280 | 0.235 | 2.5419 | 1657 | 5 |
| 36 | 35.740 | 0.259 | 2.5102 | 2536 | 8 |
| 37 | 36.480 | 0.282 | 2.4610 | 1178 | 4 |
| 38 | 37.620 | 0.235 | 2.3890 | 1136 | 4 |
| 39 | 38.740 | 0.306 | 2.3225 | 2652 | 8 |
| 40 | 39.240 | 0.282 | 2.2940 | 1743 | 6 |
| 41 | 40.520 | 0.329 | 2.2244 | 1307 | 4 |
| 42 | 42.080 | 0.259 | 2.1455 | 1122 | 4 |
| 43 | 44.100 | 0.165 | 2.0518 | 1351 | 4 |
| 44 | 44.900 | 0.259 | 2.0171 | 1865 | 6 |
| 45 | 46.600 | 0.306 | 1.9474 | 1704 | 5 |

TABLE 6B
Peak table for XRPD spectrum in FIG.
1B of crystalline HCl salt 2a

| | | | | | Height | | Area | |
|---|---|---|---|---|---|---|---|---|
| # | 2-Theta | d(?) | BG | Height | % | Area | % | FWHM |
| 1 | 7.002 | 12.6148 | 43 | 97 | 12.7 | 1544 | 13.9 | 0.271 |
| 2 | 9.718 | 9.0934 | 29 | 83 | 10.8 | 1751 | 15.8 | 0.359 |
| 3 | 11.119 | 7.9511 | 24 | 28 | 3.7 | 577 | 5.2 | 0.350 |
| 4 | 13.420 | 6.5923 | 22 | 90 | 11.8 | 1549 | 14.0 | 0.293 |
| 5 | 14.380 | 6.1543 | 23 | 141 | 18.4 | 1872 | 16.9 | 0.226 |
| 6 | 16.119 | 5.4941 | 21 | 765 | 100.0 | 11075 | 100.0 | 0.246 |
| 7 | 16.798 | 5.2735 | 34 | 56 | 7.3 | 448 | 4.0 | 0.136 |
| 8 | 17.741 | 4.9953 | 22 | 236 | 30.8 | 3570 | 32.2 | 0.257 |
| 9 | 18.703 | 4.7404 | 22 | 150 | 19.6 | 2180 | 19.7 | 0.247 |
| 10 | 19.219 | 4.6142 | 22 | 88 | 11.5 | 1408 | 12.7 | 0.272 |
| 11 | 19.838 | 4.4718 | 50 | 92 | 12.0 | 1208 | 10.9 | 0.223 |
| 12 | 20.598 | 4.3085 | 20 | 326 | 42.6 | 5806 | 52.4 | 0.303 |
| 13 | 21.763 | 4.0804 | 22 | 100 | 13.1 | 2051 | 18.5 | 0.349 |
| 14 | 22.260 | 3.9903 | 20 | 58 | 7.6 | 1589 | 14.3 | 0.466 |
| 15 | 24.479 | 3.6334 | 28 | 158 | 20.7 | 3954 | 35.7 | 0.425 |
| 16 | 25.162 | 3.5363 | 51 | 451 | 59.0 | 6914 | 62.4 | 0.261 |
| 17 | 26.198 | 3.3988 | 37 | 77 | 10.1 | 811 | 7.3 | 0.179 |
| 18 | 26.839 | 3.3190 | 37 | 71 | 9.3 | 899 | 8.1 | 0.215 |
| 19 | 27.758 | 3.2112 | 22 | 208 | 27.2 | 5600 | 50.6 | 0.458 |
| 20 | 29.060 | 3.0702 | 28 | 38 | 5.0 | 901 | 8.1 | 0.403 |
| 21 | 29.384 | 3.0371 | 25 | 39 | 5.1 | 1265 | 11.4 | 0.551 |
| 22 | 30.179 | 2.9589 | 26 | 44 | 5.8 | 714 | 6.4 | 0.276 |
| 23 | 30.919 | 2.8897 | 23 | 33 | 4.3 | 495 | 4.5 | 0.255 |
| 24 | 32.114 | 2.7849 | 26 | 30 | 3.9 | 459 | 4.1 | 0.260 |
| 25 | 32.670 | 2.7387 | 28 | 26 | 3.4 | 168 | 1.5 | 0.110 |
| 26 | 33.698 | 2.6575 | 24 | 30 | 3.9 | 535 | 4.8 | 0.303 |
| 27 | 35.454 | 2.5298 | 30 | 32 | 4.2 | 537 | 4.8 | 0.285 |
| 28 | 36.020 | 2.4914 | 29 | 45 | 5.9 | 1008 | 9.1 | 0.381 |
| 29 | 38.939 | 2.3111 | 22 | 66 | 8.6 | 1441 | 13.0 | 0.371 |
| 30 | 39.435 | 2.2831 | 20 | 38 | 5.0 | 715 | 6.5 | 0.320 |
| 31 | 40.719 | 2.2140 | 24 | 26 | 3.4 | 393 | 3.5 | 0.257 |
| 32 | 43.862 | 2.0624 | 22 | 28 | 3.7 | 442 | 4.0 | 0.268 |
| 33 | 45.081 | 2.0094 | 23 | 29 | 3.8 | 342 | 3.1 | 0.200 |
| 34 | 46.898 | 1.9357 | 21 | 37 | 4.8 | 576 | 5.2 | 0.265 |

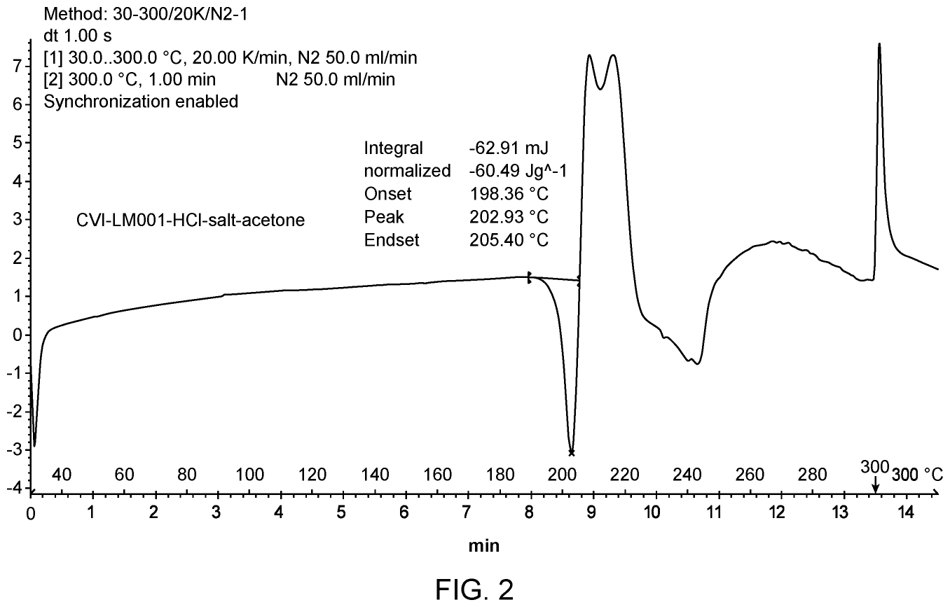

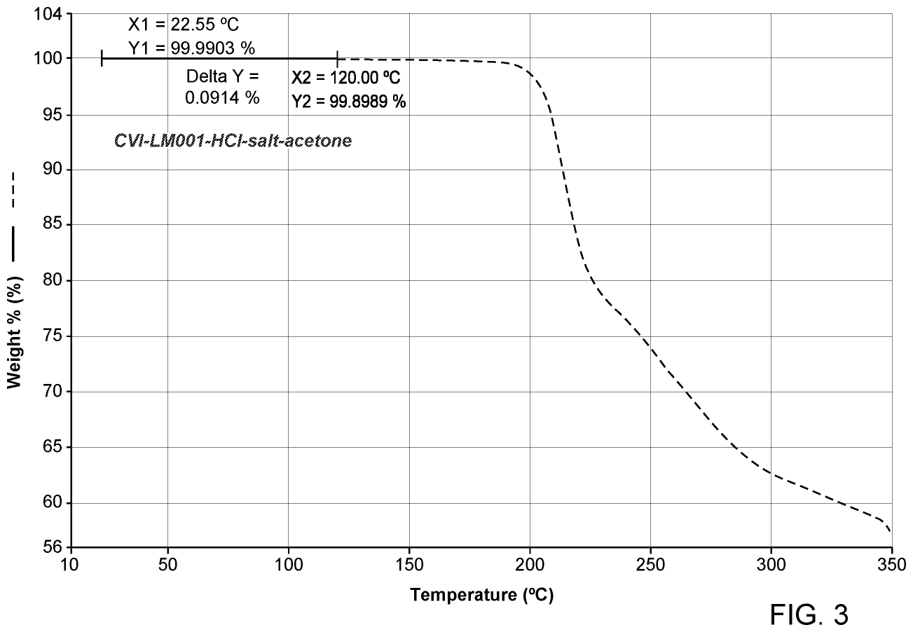

DSC and TGA: Based on the XRPD results, 2a showed a good crystallinity with sharp peaks pattern, which was consistent with PLM result. 2a did not undergoing a melting. There is no weight loss until about 150° C. DSC (FIG. 2) had a sharp endothermic peak at the onset of 196.44° C. like the melting peak. TGA (FIG. 3) results showed 2a had almost no weight loss from room temperature (RT) to 120° C.

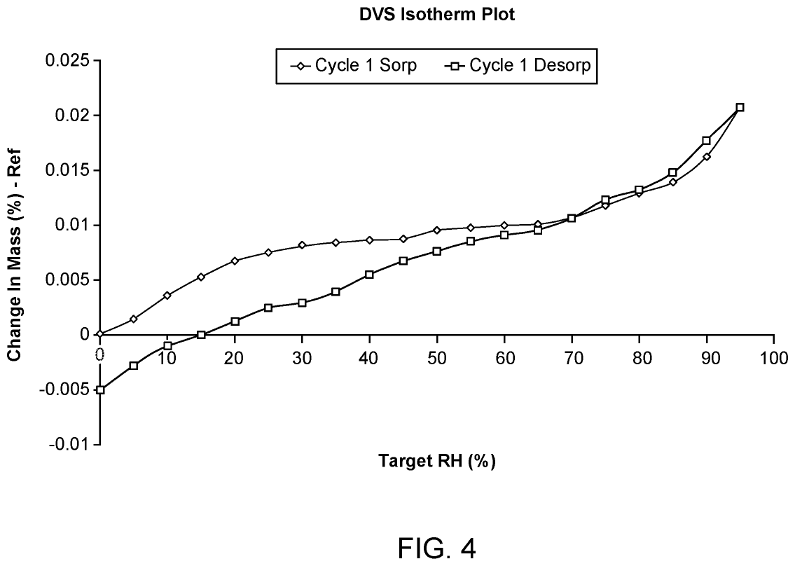

DVS Hygroscopicity: 2a was non-hygroscopic; it picks up about 0.02% of moisture at 95% RH as shown in FIG. 4 and Table 7 below.

TABLE 7
DVS Isotherm Analysis Report for 2a
Temp: 25.0° C.
MRef: 17.8268 from Mass at end of first 0% RH stage

| | Target | Change In Mass (%) - ref | | |
|---|---|---|---|---|
| | RH (%) | Sorption | Desorption | Hysteresis |
| Cycle 1 | 0.0 | 0.00011 | −0.00505 | |
| | 5.0 | 0.00146 | −0.00280 | −0.00426 |
| | 10.0 | 0.00359 | −0.00101 | −0.00460 |
| | 15.0 | 0.00527 | 0.00000 | −0.00527 |
| | 20.0 | 0.00673 | 0.00123 | −0.00550 |
| | 25.0 | 0.00752 | 0.00247 | −0.00505 |
| | 30.0 | 0.00819 | 0.00292 | −0.00527 |
| | 35.0 | 0.00841 | 0.00393 | −0.00449 |
| | 40.0 | 0.00864 | 0.00550 | −0.00314 |
| | 45.0 | 0.00875 | 0.00673 | −0.00202 |
| | 50.0 | 0.00954 | 0.00763 | −0.00191 |
| | 55.0 | 0.00976 | 0.00853 | −0.00123 |
| | 60.0 | 0.00998 | 0.00909 | −0.00090 |
| | 65.0 | 0.01010 | 0.00954 | −0.00056 |
| | 70.0 | 0.01066 | 0.01066 | 0.00000 |
| | 75.0 | 0.01178 | 0.01234 | 0.00056 |
| | 80.0 | 0.01290 | 0.01324 | 0.00034 |
| | 85.0 | 0.01391 | 0.01481 | 0.00090 |
| | 90.0 | 0.01627 | 0.01773 | 0.00146 |
| | 95.0 | 0.02076 | 0.02076 | |

Solubility: 2a substance was equilibrated with organic solvents to visually test the apparent solubility. The experiment was terminated once or if the target of 20 mg/mL was achieved. Solubilities observed are tabulated in Table 6 below. The crystalline polymorph hydrochloride salt of formula I (2a) dissolved well in some organic solvents, the solubility in methanol (MeOH) was more than 17 mg/ml, and in acetonitrile (ACN) the solubility was 6.17˜18.50 mg/ml.

TABLE 8
Solubility in organic solvents

| Solvents | Solubility (mg/ml) |
|---|---|
| EtOH | 2.10~3.15 |
| 50% EtOH (aq) | <0.99 |
| Acetone | 2.22~3.33 |
| ACN | 6.17~18.5 |
| Ethyl Acetate | <1.80 |
| MeOH | >17.32 |
| THF | <1.14 |
| Water | <1.08 |
| Heptane | <1.43 |

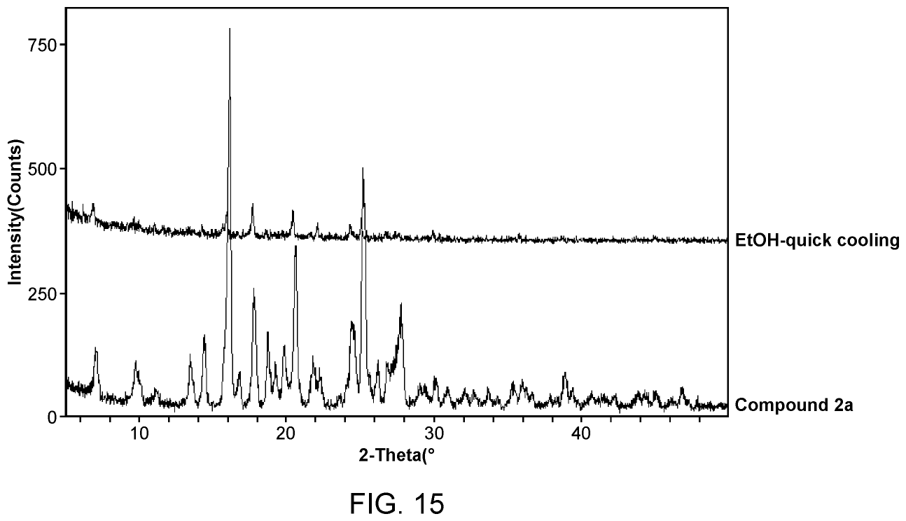

Crystallization from hot saturated solutions: About 100 mg 2a was dissolved with minimal amount of solvent at about 60° C. The solution was filtrated and separated into two portions. One portion was put in ice bath and agitated till the end of the experiment; the other portion was cooled down naturally. The precipitates were collected on a filter, dried and analyzed by XRPD. As the results in Table 9 below show, the crystalline polymorph hydrochloride salt of 2a transformed to partially crystalline when crystallization from hot saturated solutions was done. FIG. 15 shows the XRPD spectra of the 2a crystalized from hot saturated ethanol overlaid on the spectra of undisturbed 2a.

TABLE 9
Crystallization from hot saturated solutions

| Solvent | Methods | Solid form |
|---|---|---|
| EtOH | quick | partially crystalline |
| | slow | no crystal, failed |

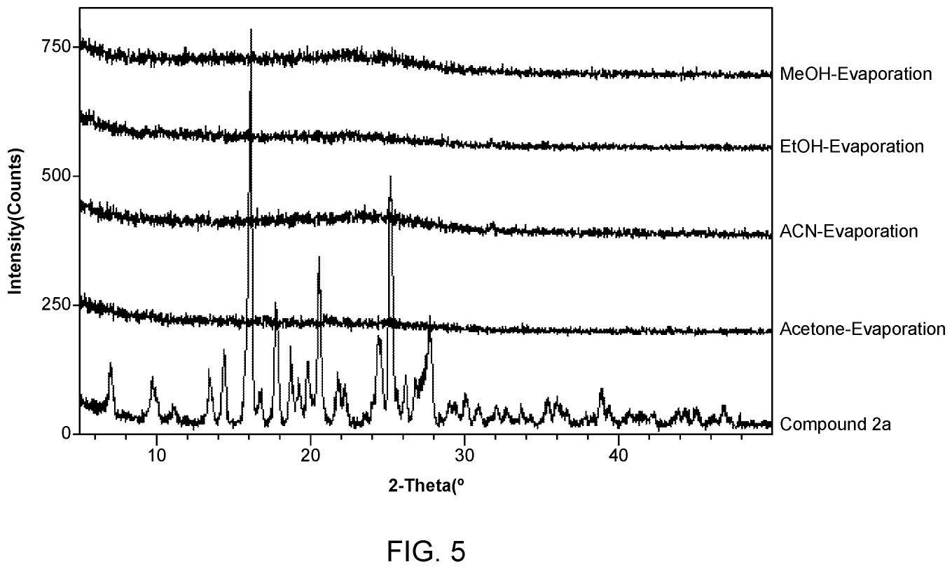

Solvent evaporation: About 20 mg 2a was dissolved in organic solvents. The solution was filtrated and dried in the air. The obtained solid part from the evaporation process was analyzed by XRPD. As shown in Table 10, after evaporation from different organic solvents, the crystal form transformed to amorphous. FIG. 5 shows the XRPD patterns of the solids left over after evaporation of each solvent in Table 10.

TABLE 10
After evaporation

| Solvents | Solid form |
|---|---|
| MeOH | Amorphous |
| Acetone | Amorphous |
| ACN | Amorphous |
| EtOH | Amorphous |

Precipitation by addition of anti-solvent: 2a was dissolved in a solvent in which 2a exhibits high solubility to get a saturated solution, then a miscible solvent in which 2a exhibits low solubility (anti-solvent) was added while stirring. The precipitates were collected and analyzed by XRPD.

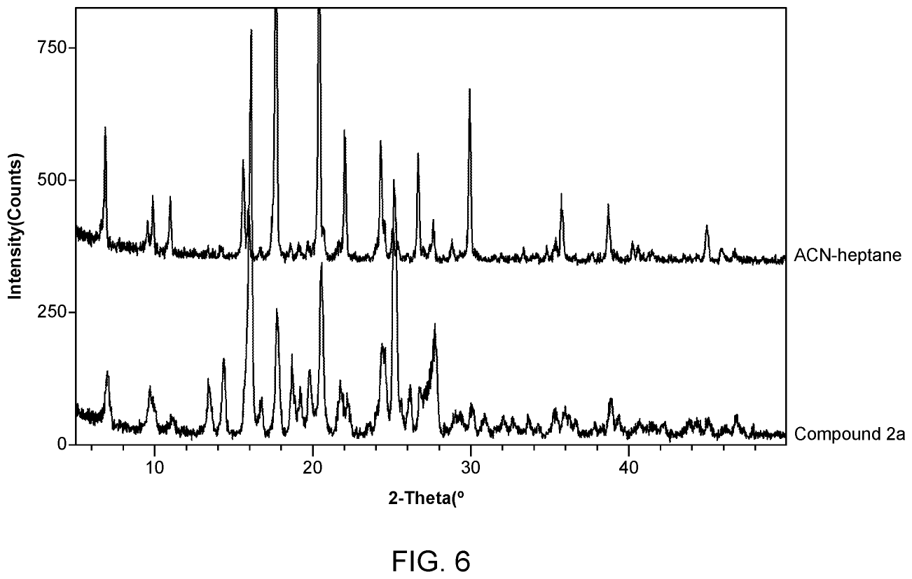

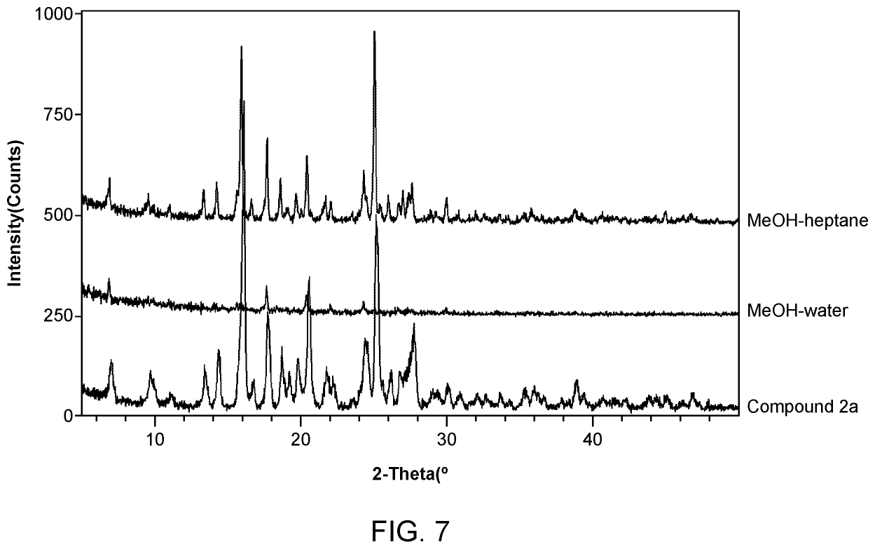

2a was dissolved in ACN or MeOH, and water or heptane was added as anti-solvent. As shown in Table 11, the crystal form was not changed when heptane was used as anti-solvent, and transformed to amorphous when water was added to MeOH solution. The XRPD pattern of compound 2a (bottom) and compound 2a dissolved in ACN then precipitated with heptane (top) is shown in FIG. 6. The XRPD pattern of compound 2a (bottom trace) and compound 2a dissolved in MeOH then precipitated with heptane (top trace) or water (center trace) is shown in FIG. 7.

TABLE 11
Precipitation by addition of anti-solvent

| Solvent | Anti-solvent | Solid form |
|---|---|---|
| ACN | Water (6)* | No crystal, failed |
| | Heptane (6) | Crystal, no change |
| MeOH | Water (2) | Amorphous |
| | Heptane (3) | Crystal, no change |

*The bracket indicates the number of volumes of anti-solvents added to the good solvent.
No change means the XRPD pattern is comparable to original 2a before the test.

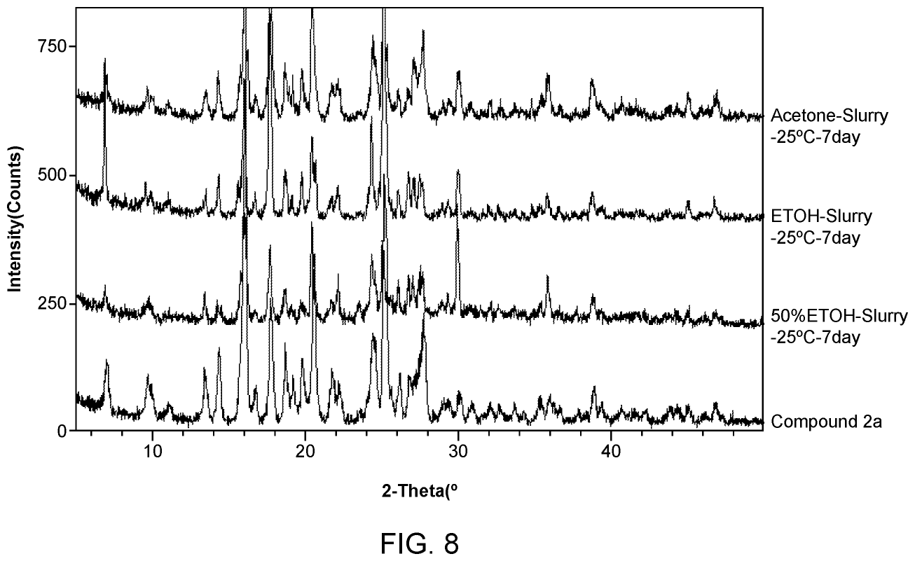

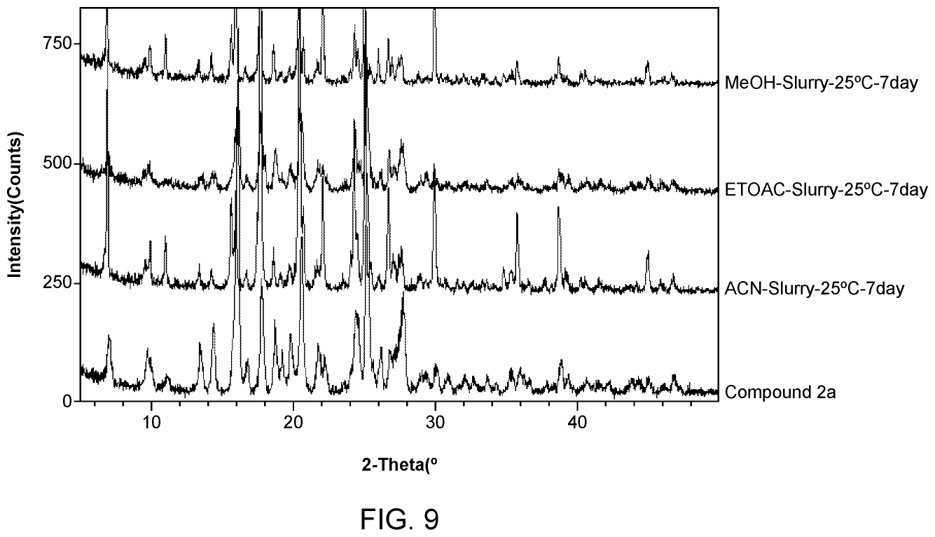

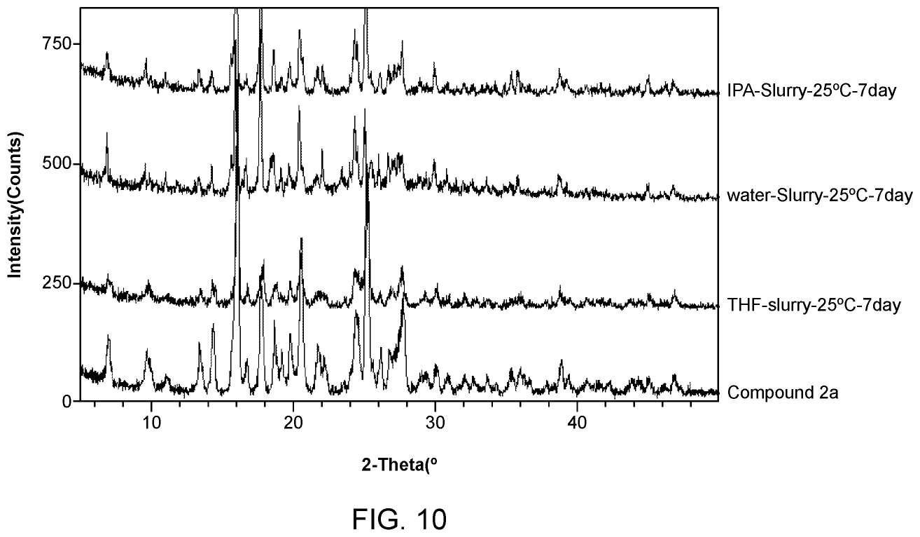

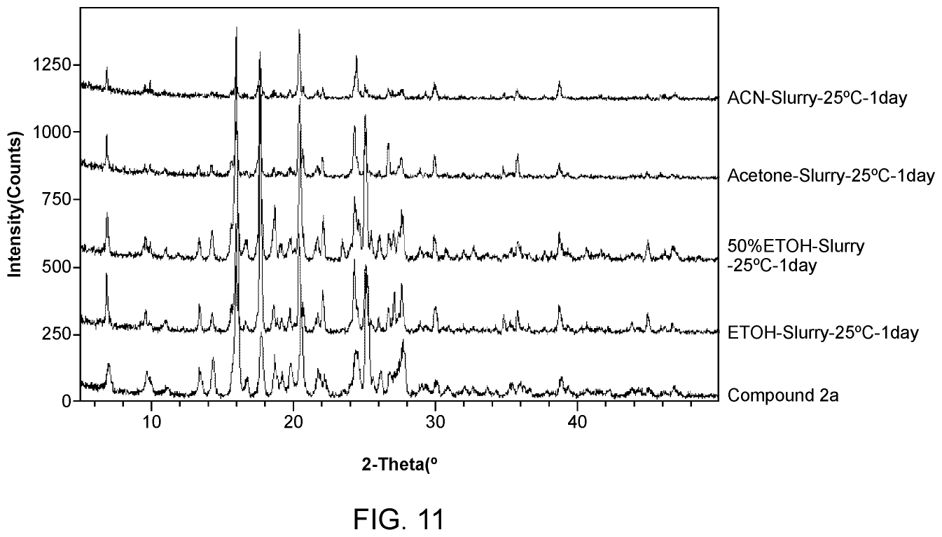

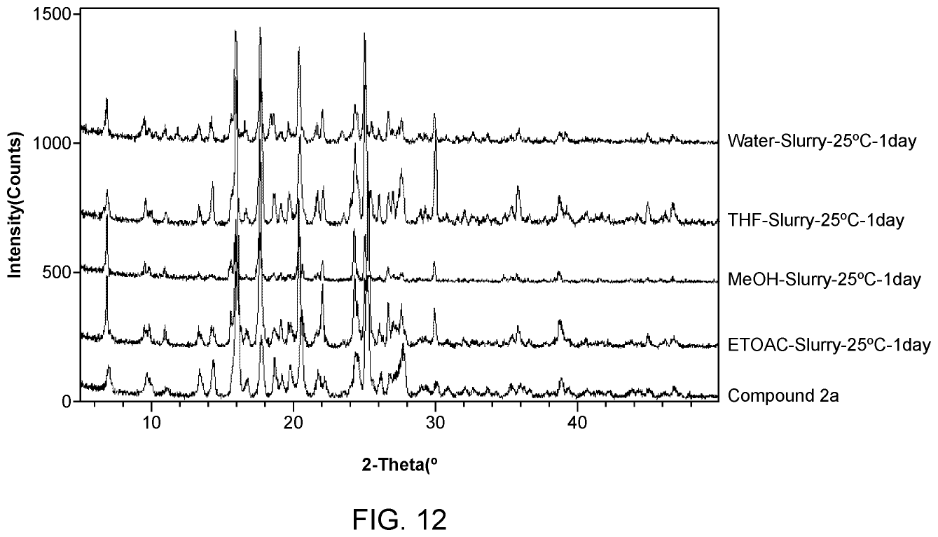

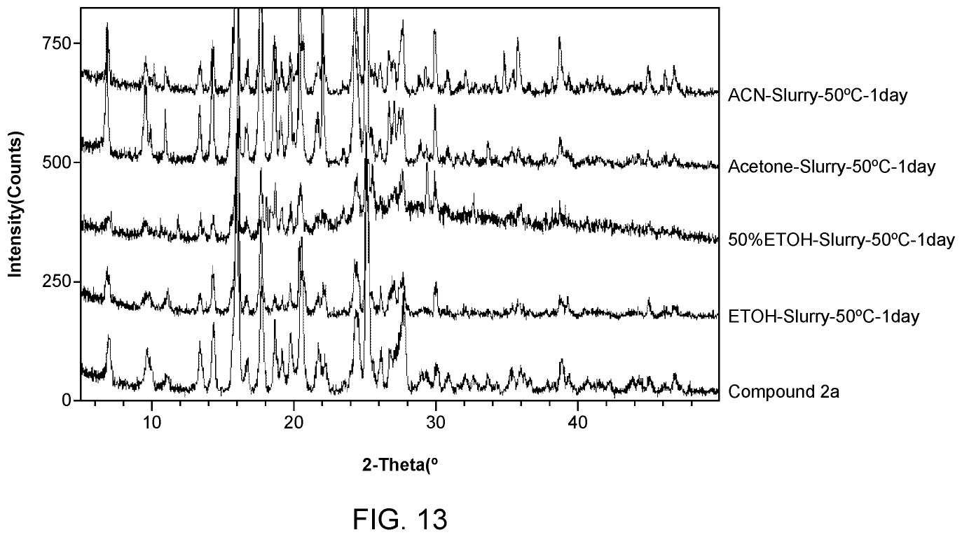

Equilibration with solvent at 25° C. and 50° C.: About 50 mg of 2a was equilibrated with 1 ml solvents at 25° C. and 50° C. After stirring for 1 day and 7 days, the suspensions were centrifuged. The solid part was collected and analyzed by XRPD, then dried in the air for 10 min and analyzed by XRPD. After equilibrium with solvent at 25° C. and 50° C. for 1 and 7 days, the crystal form was not changed. As shown in Table 12, in most conditions the solid form of comparable XRPD pattern were obtained; indicating solid form stability of 2a. The XRPD spectra of the solid equilibrated with each solvent listed in Table 12, at 25° C. and 50° C., are shown overlaid that of undisturbed 2a in FIG. 8 through FIG. 14.

TABLE 12
Equilibration of 2a with solvents at 25° C. and 50° C.
Slurry

| Solvent | 25° C.-1 day | 50° C.-1 day | 25° C.-7 day |
|---|---|---|---|
| EtOH | Crystal, no change | Crystal, no change | Crystal, no change |
| 50% | Crystal, no change | partially crystalline | Crystal, no change |
| EtOH | | | |
| (aq) | | | |
| Acetone | Crystal, no change | Crystal, no change | Crystal, no change |
| ACN | Crystal, no change | Crystal, no change | Crystal, no change |
| EtOAc | Crystal, no change | Crystal, no change | Crystal, no change |
| MeOH | Crystal, no change | Clear solution | Crystal, no change |
| THF | Crystal, no change | Crystal, no change | Crystal, no change |
| Water | Crystal, no change | partially crystalline | Crystal, no change |
| IPA | Crystal, no change | — | Crystal, no change |

No change means the XRPD pattern is comparable to lot 20110101.
—: not analyzed.

Solid stability: 5 mg of 2a was weighed into scintillation vials sealed properly and stored at 40° C./0% RH(closed), 40° C./75% RH(open) under dark conditions to avoid the effect of light on stability. Samples were collected at day 0, 5 and 10 and analyzed by HPLC. As shown in Table 13, 2a appears to have excellent solid-state stability as determined by HPLC with UV detection according to the parameters in Table 14 below. 2a is substantially stable at both conditions for up to 10 days.

TABLE 14
HPLC parameters

| Equipment | Agilent 1100 | | |
|---|---|---|---|
| Mobile phase | A: Water (with 0.05% TFA) | | |
| | B: ACN (with 0.05% TFA) | | |
| Column | Agilent Eclipse XDB C18 (5 μm, 4.6 × | | |
| | 150 mm) | | |
| | Time (min) | Phase A (%) | Phase B (%) |
| Timetable | 0 | 90 | 10 |
| | 10 | 20 | 80 |
| | 15 | 20 | 80 |
| | 15.1 | 90 | 10 |
| | 20 | 90 | 10 |
| UV Detector, nm | | 210 | |
| Injection volume, μL | | 5 | |
| Column temperature, ° C. | | 30 | |
| Flow rate, mL/min | | 1.0 | |
| Run time, min | | 20 | |

TABLE 15
Solid stability

| | | 0 day % | 5 day % | 10 day % |
|---|---|---|---|---|
| Sample | | remaining | remaining | remaining |
| Compound 2a | 40° C. | 99.65 | 101.65 | 100.85 |
| | 40° C./75% RH | 99.65 | 100.66 | 101.31 |

### Example 3: Hydrochloric Acid Polymorph (2b)

Preparation of crystalline HCl polymorph (2b): About 200 mg of 1b was dissolved in 3 ml Acetone at about 50˜60° C. and the solution was clear. 0.6 mL of 1.0 mol/L HCl was added dropwise to the solution with stirring at 50˜60° C. Then it was cooled to RT slowly, white powder precipitated out quickly. The solid part was filtered and dried. 2.5 ml of 95% EtOH was added with stirred at 50˜60° C., and the solid dissolved. Then it was cooled to RT slowly, white crystal precipitated out. The solid part was filtered and dried, to yield crystalline HCl salt 2b.

XRPD: The XRPD result of 1b showed it was amorphous as provide in FIG. 21 of freebase 1b (top) overlaid that of 2b (bottom).

DSC and TGA: There is no obvious weight loss until about 200° C. in the case of 2b (FIG. 18B) and no melting observed. TGA of the compound (FIG. 19B) confirms the conclusion from DSC.

DVS Hygroscopicity: FIG. 20B and Table 16 showed that 2b was not hygroscopic, it picks up ˜0.1% of moisture at 95% RH.

TABLE 16
DVS results for 2b

| | Target | Change In Mass (%) - ref | | |
|---|---|---|---|---|
| | RH (%) | Sorption | Desorption | Hysteresis |
| Cycle 1 | 0.0 | 0.0011 | −0.0081 | |
| | 5.0 | 0.0007 | −0.0035 | −0.0042 |
| | 10.0 | 0.0032 | 0.0000 | −0.0032 |
| | 15.0 | 0.0044 | 0.0011 | −0.0033 |
| | 20.0 | 0.0081 | 0.0044 | −0.0037 |
| | 25.0 | 0.0099 | 0.0062 | −0.0037 |
| | 30.0 | 0.0118 | 0.0081 | −0.0037 |
| | 35.0 | 0.0134 | 0.0100 | −0.0033 |
| | 40.0 | 0.0150 | 0.0123 | −0.0026 |
| | 45.0 | 0.0173 | 0.0185 | 0.0012 |
| | 50.0 | 0.0169 | 0.0204 | 0.0035 |
| | 55.0 | 0.0187 | 0.0243 | 0.0056 |
| | 60.0 | 0.0201 | 0.0264 | 0.0063 |
| | 65.0 | 0.0211 | 0.0294 | 0.0083 |
| | 70.0 | 0.0270 | 0.0344 | 0.0074 |
| | 75.0 | 0.0352 | 0.0382 | 0.0030 |
| | 80.0 | 0.0409 | 0.0446 | 0.0037 |
| | 85.0 | 0.0500 | 0.0530 | 0.0030 |
| | 90.0 | 0.0638 | 0.0689 | 0.0051 |
| | 95.0 | 0.0941 | 0.0941 | |

Counterion analysis: The freebase 1b was dissolved in water to prepare a calibration curve. HCl was dissolved in water to prepare a calibration curve. The sample of 2b was dissolved in MeOH and analyzed by HPLC for counterion concentration. HPLC data analyses was acquired using the parameters of Table 17.

TABLE 17
HPLC conditions for analysis of solution of 2b
Conditions

| Equipment | Agilent 1200 |
|---|---|
| Mobile phase | 50% ACN with 0.1M HCOONH₄ pH = 4.7 |
| Column | Phenosphere SAX 5μ 250*4.6 mm |
| ELSD Detector, T | 40° C. |
| Injection volume, μL | 20 |
| Column temperature, ° C. | 30 |
| Flow rate, mL/min | 1.0 |
| Run time, min | 10 |

Two samples of 2b were analyzed (Table 18) and the HCl salt was shown to be 1:1 stoichiometric.

TABLE 18
Counter-ion analysis of HCl salt of compound 1b

| Samples | Theoretical conc. (ug/ml) | Area | Measured conc. (ug/ml) |
|---|---|---|---|
| sample 1 | 500 | 292147 | 597.8 |
| sample 2 | 462 | 223038 | 456.4 |

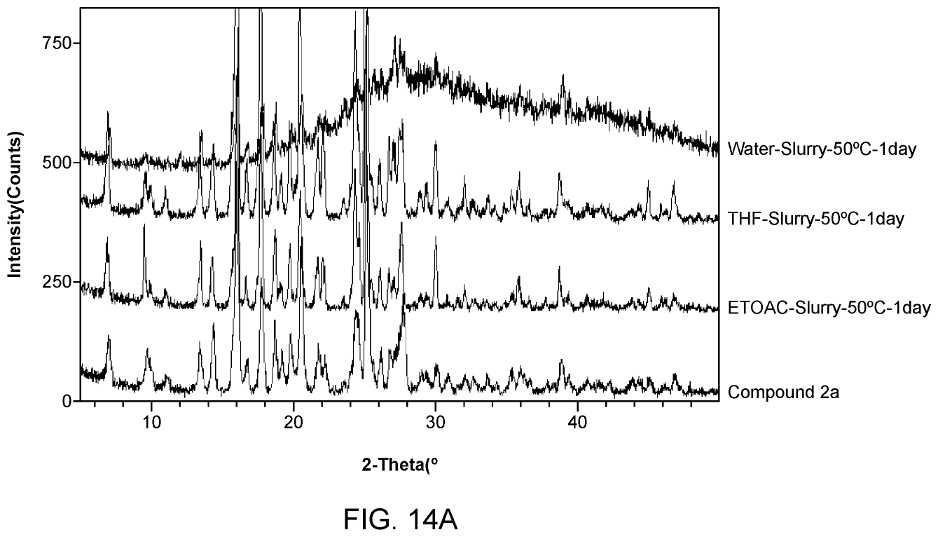

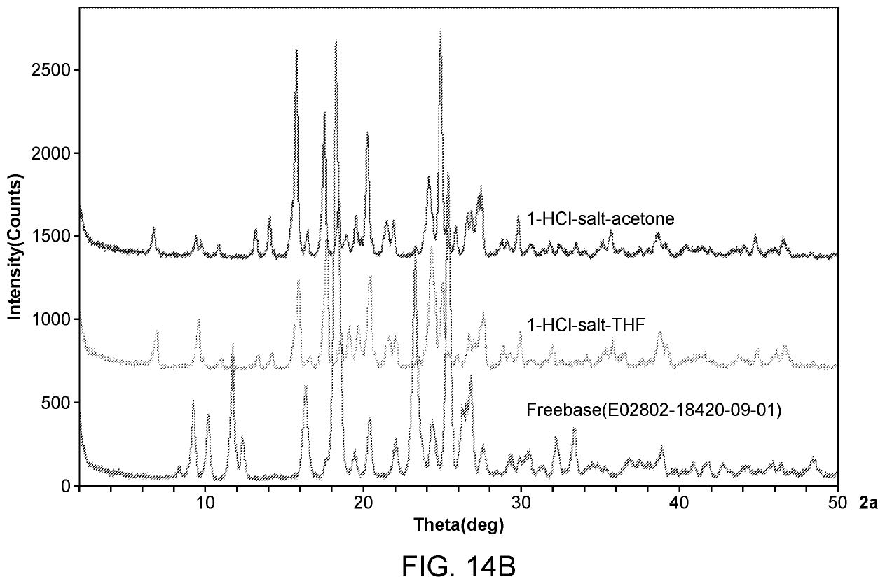

Hydrochloride salt of compound 1a was readily prepared and could be obtained from a variety of solvent systems (e.g., THF and acetone) (FIG. 14B). DSC showed that the melting point of hydrochloric acid was 198.36° C. (onset), TGA had almost no weight loss from RT to 120° C., indicating that there was almost no residual solvent or water. The hydrochloride is an excellent salt for formulation development of both compounds 1a and 1b.

### Example 4: Mesylate Polymorph (3b)

Preparation of crystalline mesylate polymorph (3b): About 100 mg of 1b was dissolved in 2 ml acetone at about 50˜60° C. and the solution was clear. 0.36 mL of 1.0 mol/L methanesulfonate was added dropwise to the solution with stirring at 50˜60° C. Then it was cooled to RT slowly, and evaporated with N₂. 1 mL IPA was added with stirring at 50˜60° C., and the solid dissolved. Then it was cooled to RT slowly, white crystal 3b precipitated out.

The XRPD spectrum of 3b (FIG. 22) was obtained according to the conditions in Table 19 and produced the peaks as tabulated therein.

TABLE 19
XRPD peak table for spectrum of FIG. 22
SCAN: 5.0/50.0/0.02/5(deg/m), Cu(40 kV,30 mA), I(max) = 3754,
PEAK: 17-pts/Parabolic Filter, Threshold = 3.0, Cutoff = 1.0%, BG = 3/1.0,
Peak-Top = Summit

| | | | | | Height | | Area | |
|---|---|---|---|---|---|---|---|---|
| # | 2-Theta | d(?) | BG | Height | % | Area | % | FWHM |
| 1 | 5.979 | 14.7697 | 57 | 311 | 8.4 | 2720 | 8.9 | 0.149 |
| 2 | 7.099 | 12.4423 | 49 | 3705 | 100.0 | 30609 | 100.0 | 0.140 |
| 3 | 8.501 | 10.3928 | 41 | 1401 | 37.8 | 13774 | 45.0 | 0.167 |
| 4 | 9.482 | 9.3199 | 36 | 182 | 4.9 | 1956 | 6.4 | 0.183 |
| 5 | 10.120 | 8.7338 | 35 | 157 | 4.2 | 1332 | 4.4 | 0.144 |
| 6 | 11.558 | 7.6497 | 27 | 109 | 2.9 | 1416 | 4.6 | 0.221 |
| 7 | 14.142 | 6.2574 | 32 | 372 | 10.0 | 4761 | 15.6 | 0.218 |
| 8 | 14.601 | 6.0619 | 3 | 832 | 22.5 | 9287 | 30.3 | 0.190 |
| 9 | 15.839 | 5.5907 | 42 | 214 | 5.8 | 1793 | 5.9 | 0.142 |
| 10 | 16.917 | 5.2367 | 50 | 1140 | 30.8 | 11966 | 39.1 | 0.178 |
| 11 | 17.679 | 5.0128 | 49 | 385 | 10.4 | 4091 | 13.4 | 0.181 |
| 12 | 18.139 | 4.8864 | 44 | 122 | 3.3 | 2030 | 6.6 | 0.283 |
| 13 | 18.843 | 4.7056 | 55 | 137 | 3.7 | 2398 | 7.8 | 0.298 |
| 14 | 19.255 | 4.6058 | 68 | 1006 | 27.2 | 10649 | 34.8 | 0.180 |
| 15 | 19.921 | 4.4532 | 75 | 2315 | 62.5 | 25568 | 83.5 | 0.188 |
| 16 | 21.076 | 4.2118 | 64 | 616 | 16.6 | 7057 | 23.1 | 0.195 |
| 17 | 21.641 | 4.1031 | 42 | 400 | 10.8 | 4740 | 15.5 | 0.201 |
| 18 | 22.483 | 3.9512 | 61 | 161 | 4.3 | 1455 | 4.8 | 0.154 |
| 19 | 23.059 | 3.8538 | 58 | 152 | 4.1 | 2399 | 7.8 | 0.268 |
| 20 | 23.520 | 3.7794 | 68 | 142 | 3.8 | 2135 | 7.0 | 0.256 |
| 21 | 23.900 | 3.7201 | 69 | 305 | 8.2 | 2754 | 9.0 | 0.154 |
| 22 | 24.979 | 3.5618 | 58 | 422 | 11.4 | 6367 | 20.8 | 0.256 |
| 23 | 25.404 | 3.5032 | 57 | 49 | 1.3 | 460 | 1.5 | 0.160 |
| 24 | 25.839 | 3.4452 | 51 | 113 | 3.0 | 1129 | 3.7 | 0.170 |
| 25 | 26.322 | 3.3830 | 43 | 59 | 1.6 | 672 | 2.2 | 0.194 |
| 26 | 27.563 | 3.2335 | 65 | 133 | 3.6 | 1070 | 3.5 | 0.137 |
| 27 | 28.441 | 3.1356 | 70 | 650 | 17.5 | 9815 | 32.1 | 0.257 |
| 28 | 29.719 | 3.0037 | 59 | 471 | 12.7 | 6886 | 22.5 | 0.249 |
| 29 | 31.601 | 2.8289 | 49 | 279 | 7.5 | 7625 | 24.9 | 0.465 |
| 30 | 32.099 | 2.7862 | 55 | 715 | 19.3 | 11342 | 37.1 | 0.270 |
| 31 | 33.161 | 2.6993 | 63 | 67 | 1.8 | 355 | 1.2 | 0.090 |
| 32 | 33.522 | 2.6711 | 54 | 160 | 4.3 | 1914 | 6.3 | 0.203 |
| 33 | 34.077 | 2.6288 | 63 | 147 | 4.0 | 1114 | 3.6 | 0.129 |
| 34 | 34.983 | 2.5628 | 54 | 116 | 3.1 | 3149 | 10.3 | 0.461 |
| 35 | 35.361 | 2.5363 | 55 | 929 | 25.1 | 12982 | 42.4 | 0.238 |
| 36 | 36.677 | 2.4482 | 57 | 389 | 10.5 | 4100 | 13.4 | 0.179 |
| 37 | 37.263 | 2.4111 | 64 | 44 | 1.2 | 439 | 1.4 | 0.170 |
| 38 | 37.842 | 2.3754 | 61 | 63 | 1.7 | 1246 | 4.1 | 0.336 |
| 39 | 38.220 | 2.3528 | 58 | 66 | 1.8 | 1004 | 3.3 | 0.259 |
| 40 | 39.284 | 2.2916 | 54 | 102 | 2.8 | 693 | 2.3 | 0.116 |
| 41 | 39.700 | 2.2685 | 48 | 46 | 1.2 | 2843 | 9.3 | 1.051 |
| 42 | 40.219 | 2.2404 | 57 | 459 | 12.4 | 6364 | 20.8 | 0.236 |
| 43 | 41.720 | 2.1632 | 51 | 103 | 2.8 | 1400 | 4.6 | 0.231 |
| 44 | 42.720 | 2.1148 | 51 | 73 | 2.0 | 1653 | 5.4 | 0.385 |
| 45 | 43.801 | 2.0651 | 48 | 218 | 5.9 | 2909 | 9.5 | 0.227 |
| 46 | 45.676 | 1.9846 | 47 | 67 | 1.8 | 815 | 2.7 | 0.207 |
| 47 | 46.720 | 1.9427 | 47 | 147 | 4.0 | 1914 | 6.3 | 0.221 |
| 48 | 48.716 | 1.8676 | 43 | 45 | 1.2 | 650 | 2.1 | 0.246 |
| 49 | 49.357 | 1.8449 | 48 | 74 | 2.0 | 687 | 2.2 | 0.158 |

NOTE:
Intensity = Counts, 2T(0) = 0.0(deg), Wavelength to Compute d-Spacing = 1.54056? (Cu/K-alpha1)

The mesylate 3a could be obtained from MEK system or other systems only after proper recrystallization because methanesulfonic acid solution was prepared with water and aqueous solution was added into the reaction system, which affected the crystallization of mesylate. Based on the results of XRPD, the diffraction peaks of mesylate obtained from different systems were different from each other, indicating that the mesylate formed different polymorphs.

DSC and TGA: The mesylate salt prepared from ethyl acetate and recrystallized by acetone and ethyl acetate were tested by TGA (FIG. 19C) and DSC (FIG. 19B). TGA showed that there was a weight loss of 1.1% from room temperature to 120° C. DSC showed that the melting point of mesylate was 145.72° C.

NMR: The results of ¹H-NMR showed that the molar ratio of mesylate was 1:1.3 (base: acid), and methanesulfonic acid was a little higher than theoretical molar ratio of 1:1.

DVS Hygroscopicity of 3b: FIG. 20C and Table 20 showed that 3b was hygroscopic, it picks up ˜13% of moisture at 80% RH, and ˜18% of moisture at 95% RH.

TABLE 20
DVS results for 3b

| | Target | Change In Mass (%) - ref | | |
|---|---|---|---|---|
| | RH (%) | Sorption | Desorption | Hysteresis |
| Cycle 1 | 0.0 | 0.00 | 0.05 | |
| | 5.0 | 0.03 | 0.26 | 0.23 |
| | 10.0 | 0.28 | 0.46 | 0.18 |
| | 15.0 | 0.32 | 0.61 | 0.29 |
| | 20.0 | 0.34 | 4.13 | 3.79 |
| | 25.0 | 0.39 | 8.89 | 8.50 |
| | 30.0 | 7.46 | 9.08 | 1.62 |
| | 35.0 | 8.93 | 9.14 | 0.21 |
| | 40.0 | 9.06 | 9.18 | 0.13 |
| | 45.0 | 9.10 | 9.26 | 0.16 |
| | 50.0 | 9.14 | 10.73 | 1.59 |
| | 55.0 | 9.22 | 10.99 | 1.77 |
| | 60.0 | 9.41 | 11.23 | 1.82 |
| | 65.0 | 11.14 | 11.50 | 0.36 |
| | 70.0 | 11.79 | 11.84 | 0.04 |
| | 75.0 | 12.23 | 12.27 | 0.04 |
| | 80.0 | 12.79 | 12.85 | 0.05 |
| | 85.0 | 13.64 | 13.71 | 0.08 |
| | 90.0 | 15.08 | 15.23 | 0.15 |
| | 95.0 | 17.97 | 17.97 | |

Counterion analysis: The freebase 1b was dissolved in water to prepare a calibration curve. Methanesulfonate was dissolved in water to prepare a calibration curve. The sample of 3b was dissolved in MeOH and analyzed by HPLC for counterion concentration. HPLC data analysis was obtained using the parameters of Table 21.

TABLE 21
HPLC conditions for analysis of solution of 3b
Conditions

| Equipment | Agilent 1200 |
|---|---|
| Mobile phase | 50% ACN with 0.1M HCOONH₄, pH = 4.6 |
| Column | Phenosphere SAX 5μ 250*4.6 mm |
| ELSD Detector, T | 40° C. |
| Injection volume, μL | 20 |
| Column temperature, ° C. | 30 |
| Flow rate, mL/min | 1.5 |
| Run time, min | 10 |

Two samples of 3b were analyzed (Table 22) and the mesylate salt was shown to be 1:1 stoichiometric.

TABLE 22
Counter-ion analysis of mesylate 3b

| Samples | Theoretical conc. (ug/ml) | Area | Measured conc. (ug/ml) |
|---|---|---|---|
| sample 1 | 1155 | 231079 | 1404 |
| sample 2 | 1222 | 224006 | 1352 |

### Example 5: Solubility and Stability of 1b, 2b, and 3b

Solubility: Solubilities were determined for 1b, 2b, and 3b and are tabulated in Table 23 below.

TABLE 23
Solubility data

| | 1b | | 2b | | 3b | |
|---|---|---|---|---|---|---|
| Fluid | conc.(mg/ml) | Final pH | conc.(mg/ml) | Final pH | conc.(mg/ml) | Final pH |
| 0.1N HCL | 0.605 | 1.68 | 0.206 | 1.59 | 0.261 | 1.56 |
| pH 4.5 | 0.008 | 4.59 | 0.010 | 4.44 | 0.012 | 4.42 |
| pH 6.8 | 0.011 | 6.91 | 0.008 | 6.67 | 0.014 | 6.64 |
| Water | 0.008 | 5.42 | 0.096 | 2.74 | 0.145 | 2.71 |
| SGF | 0.588 | 1.67 | 0.147 | 1.59 | 1.828 | 1.55 |
| FaSSIF | 0.007 | 6.52 | 0.010 | 6.23 | 0.009 | 6.04 |
| FeSSIF | 0.009 | 5.14 | 0.011 | 4.93 | 0.010 | 4.87 |

* the solubility of salts was transformed to freebase.
Media:
pH 4.5 Acetate Buffer (USP)
pH 6.8 Phosphate Buffer (USP)
SGF: Simulated Gastric Fluid
FaSSIF: Simulated Small Intestinal Fluid under Fasted state
FeSSIF: Simulated Small Intestinal Fluid under Fed state

Solution stability: A stock solution of compound (1.0 mg/mL) in MeOH was diluted with 0.1 N HCl, pH 4.5 buffer (acetate) and pH 6.8 buffer to reach constant ionic strength of 0.15 M and the same final concentration (50 μg/mL, 40% MeOH).

A stock solution of 1b and 3b (1.0 mg/mL) in MeOH was diluted with pH 6.8 buffer to reach constant ionic strength of 0.15M and the same final concentration (25 μg/mL, 40% DMSO).

The solutions were stored at 25° C. and 37° C. At predetermined time points (0, 3, 24 h), the samples were withdrawn and analyzed for potency/degradation by HPLC. The results are tabulated in Table 24 and Table 25 below.

TABLE 24
Remaining % of 1b, 2b, and 3b stored at 25° C. in different pH buffers for 24 hours

| | 1b | | | 2b | | | 3b | | |
|---|---|---|---|---|---|---|---|---|---|
| Time (h) | 0.1N HCL | pH 4.5 | pH 6.8* | 0.1N HCL | pH 4.5 | pH 6.8 | 0.1N HCL | pH 4.5 | pH 6.8* |
| 0 | 100.00 | 100.00 | 100.00 | 100.00 | 100.00 | 100.00 | 100.00 | 100.00 | 100.00 |
| 3 | 100.53 | 99.94 | 99.18 | 100.07 | 99.80 | 99.52 | 100.31 | 100.00 | 99.64 |
| 24 | 100.88 | 98.79 | 91.23 | 100.37 | 99.52 | 98.01 | 100.62 | 100.08 | 99.64 |

*the final conc. was 25 μg/mL with 40% DMSO.

2b and 3b were stable 24 h at 25° C. 1b was stable at 0.1N HCl, pH 4.5 buffer in 24 h at 25° C., but clear degradations were observed at pH 6.8 buffer.

TABLE 25
Remaining % of 1b, 2b, and 3b stored at 37° C. in different pH buffers for 24 hours

| | 1b | | | 2b | | | 3b | | |
|---|---|---|---|---|---|---|---|---|---|
| Time (h) | 0.1N HCL | pH 4.5 | pH 6.8* | 0.1N HCL | pH 4.5 | pH 6.8 | 0.1N HCL | pH 4.5 | pH 6.8* |
| 0 | 100.00 | 100.00 | 100.00 | 100.00 | 100.00 | 100.00 | 100.00 | 100.00 | 100.00 |
| 3 | 101.92 | 99.51 | 98.97 | 100.34 | 100.00 | 100.28 | 99.92 | 100.39 | 99.22 |
| 24 | 100.14 | 98.83 | 94.70 | 100.95 | 99.45 | 99.86 | 100.92 | 100.47 | 85.89 |

*the final conc. was 25 μg/mL with 40% DMSO.

2b was stable in 24 h at 37° C. 1b and 3b were stable at 0.1N HCl, pH 4.5 buffer in 24 h at 37° C. but decomposed at pH 6.8 buffer. Overall, 2b had the best solution stability behaviors.

Solid stability: 5 mg of compound was weighed into vials sealed properly and stored at 40° C./0% RH (closed), 40° C./75% RH (open) under dark conditions to avoid the effect of light. Samples were analyzed by HPLC at days 0, 7, and 21. Results are shown below in Table 26.

TABLE 26
Solid stability results

| | 0 day % | 7 day % | 21 day % |
|---|---|---|---|
| Sample | remaining | remaining | remaining |

| | | | | |
|---|---|---|---|---|
| 1b | 40° C. | 100.0 | 99.3 | 98.4 |
| | 40° C./75% RH | 100.0 | 99.2 | 102.0 |
| 3b | 40° C. | 100.0 | 99.1 | 99.2 |
| | 40° C./75% RH | 100.0 | 100.3 | 99.8 |
| 2b | 40° C. | 100.0 | 100.4 | 100.0 |
| | 40° C./75% RH | 100.0 | 100.3 | 100.1 |

1b, 2b, and 3b appear to have excellent solid-state stability. They are substantially stable at both conditions for up to 3 weeks.

Solid stability of compound 1a: 5 mg compound were accurately weighed into scintillation vials sealed properly and stored at 40° C./0% RH(closed), 40° C./75% RH(open) under dark condition to avoid the effect of light on stability. Samples should be collected at day 0, 5 and 10 days and analyzed by HPLC.

TABLE 27
Solid stability

| | | 0 day % | 5 day % | 10 day % |
|---|---|---|---|---|
| Sample | | remaining | remaining | remaining |
| Compound 1a | 40° C. | 99.65 | 101.65 | 100.85 |
| | 40° C./75% RH | 99.65 | 100.66 | 101.31 |

Compound 1a appears to have excellent solid-state stability. It is substantially stable at both conditions for up to 10 days.

Additional stability test results in Table 28 showed that the compound was stable up to 36 months under conditions of 30° C./65% RH.

TABLE 28
Long term stability of Compound 1a

| | % Remaining | | | | | | | |
|---|---|---|---|---|---|---|---|---|
| | | 1 | 3 | 6 | 12 | 18 | 24 | 36 |
| Time Period | 0 | month | month | month | month | month | month | month |
| Cmpd 1a | 100.10% | 100.30% | 100.00% | 99.30% | 99.60% | 99.20% | 99.50% | 100.40% |

### Example 6: Sulfate Polymorph of Compound 1a

Preparation of crystalline sulfate polymorph (Formula X): 1.0 g of freebase was weighed into a round bottom flask, 20 mL of ethyl acetate was added, and sonicated a few minutes to form a uniform suspension. Most of the freebase was dissolved visually. At room temperature, 2236 μL of sulfuric acid solution (1 mol/L in ethyl acetate) was added into the flask. The sample was stirred continuously for 24 hours. Preparation of 1 mol/L of sulfuric acid solution: take 1.111 mL concentrated sulfuric acid, dissolve it with ethyl acetate, dilute it to 20 mL by a volumetric flask, and shake well. The suspension was filtered to obtain a white wet cake, which was washed by ethyl acetate once, and dried in vacuum oven at 40° C. for 4 hours. 1.149 g of the sulfate polymorph was collected (yield was 95%).

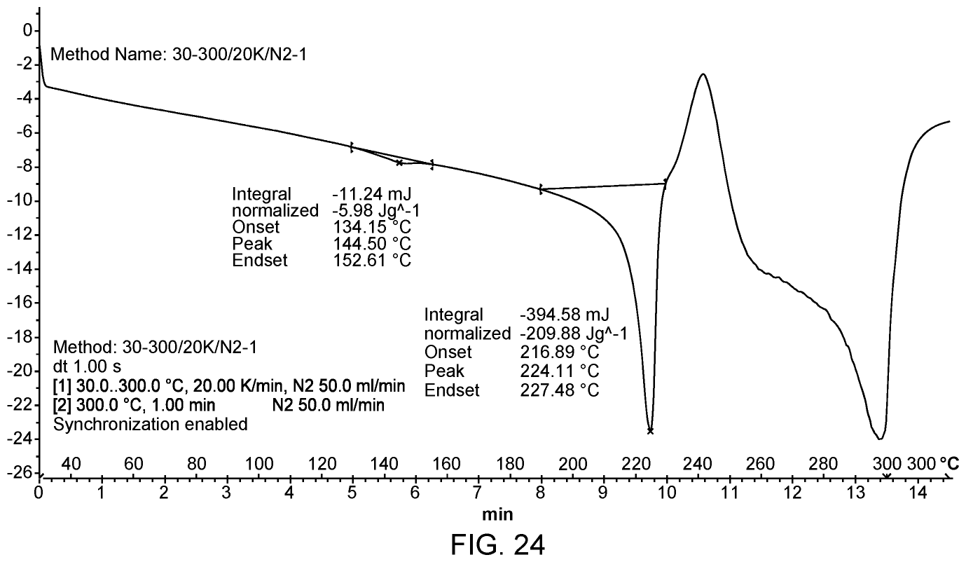

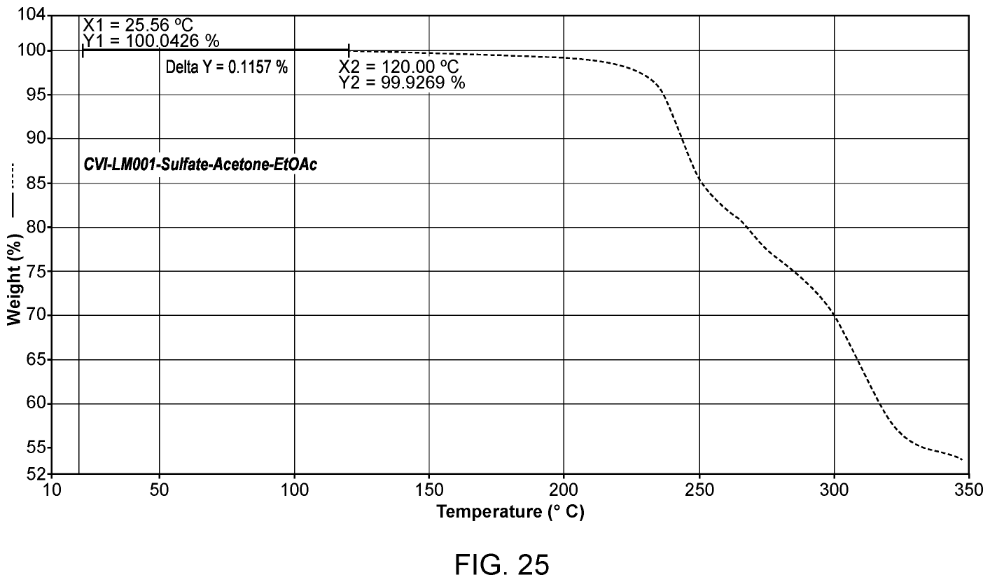

It was difficult to obtain sulfate directly from some solvent systems in the small-scale of salt screening. The results of XRPD (Table 29 and FIG. 23) showed that sulfate was prepared by different solvent systems, with only one crystal form and good crystallinity. Sulfate salt which was obtained from acetone and recrystallized in ethyl acetate was as an example. TGA showed no weight loss from room temperature to 120° C. (See FIG. 25.) DSC showed that the melting point of sulfate was 216.89° C. (Onset). (See FIG. 24.)

TABLE 29
Peak Search Report (29 Peaks, Max P/N = 18.9)
[3_2-Sulfate-Acetone-EtOAc.RAW] Sulfate-Acetone-EtOAc
PEAK: 23-pts/Parabolic Filter, Threshold = 1.0, Cutoff = 2.0%, BG = 3/1.0,
Peak-Top = Summit

| 2-Theta | d(?) | BG | Height | I % | Area | I % | FWHM |
|---|---|---|---|---|---|---|---|
| 7.424 | 11.8986 | 53 | 101 | 6.5 | 1475 | 3.9 | 0.248 |
| 12.278 | 7.2027 | 51 | 465 | 30 | 6749 | 17.8 | 0.247 |
| 13.6 | 6.5054 | 54 | 244 | 15.8 | 5128 | 13.5 | 0.357 |
| 14.08 | 6.2848 | 93 | 205 | 13.2 | 10140 | 26.7 | 0.841 |
| 14.459 | 6.121 | 59 | 767 | 49.5 | 16866 | 44.4 | 0.374 |
| 14.961 | 5.9166 | 59 | 155 | 10 | 1870 | 4.9 | 0.205 |
| 17.321 | 5.1155 | 59 | 455 | 29.4 | 10223 | 26.9 | 0.382 |
| 17.9 | 4.9514 | 109 | 325 | 2 | 6703 | 17.6 | 0.351 |
| 18.733 | 4.7328 | 119 | 97 | 6.3 | 1135 | 3 | 0.199 |
| 19.261 | 4.6045 | 101 | 161 | 10.4 | 1554 | 4.1 | 0.164 |
| 20.022 | 4.4311 | 87 | 129 | 8.3 | 3447 | 9.1 | 0.454 |
| 20.478 | 4.3334 | 88 | 106 | 6.8 | 3447 | 9.1 | 0.553 |
| 21.42 | 4.1449 | 118 | 272 | 17.6 | 5343 | 14.1 | 0.334 |
| 21.758 | 4.0813 | 136 | 136 | 8.8 | 3965 | 10.4 | 0.496 |
| 22.82 | 3.8937 | 133 | 1549 | 100 | 38009 | 100 | 0.417 |
| 24.721 | 3.5984 | 131 | 865 | 55.8 | 17599 | 46.3 | 0.346 |
| 25.159 | 3.5368 | 130 | 196 | 12.7 | 5296 | 13.9 | 0.459 |
| 26.019 | 3.4217 | 132 | 92 | 5.9 | 1656 | 4.4 | 0.306 |
| 26.48 | 3.3632 | 111 | 245 | 15.8 | 7227 | 19 | 0.501 |
| 29.058 | 3.0704 | 73 | 105 | 6.8 | 1698 | 4.5 | 0.275 |
| 30.541 | 2.9246 | 96 | 104 | 6.7 | 2251 | 5.9 | 0.368 |
| 31.64 | 2.8255 | 96 | 104 | 6.7 | 1703 | 4.5 | 0.278 |
| 34.186 | 2.6207 | 66 | 42 | 2.7 | 1521 | 4 | 0.616 |
| 35.281 | 2.5418 | 68 | 38 | 2.5 | 1683 | 4.4 | 0.753 |
| 36.402 | 2.4661 | 70 | 46 | 3 | 1264 | 3.3 | 0.467 |
| 37.559 | 2.3927 | 70 | 68 | 4.4 | 1523 | 4 | 0.381 |
| 43.781 | 2.066 | 67 | 29 | 1.9 | 1056 | 2.8 | 0.619 |
| 44.278 | 2.044 | 65 | 43 | 2.8 | 1106 | 2.9 | 0.437 |
| 47.143 | 1.9262 | 66 | 46 | 3 | 1099 | 2.9 | 0.406 |

### Example 7: Besylate Polymorph of Compound 1a

Preparation of crystalline besylate polymorph: The besylate polymorph was obtained by recrystallization in ethyl acetate by slurry method. The results of XRPD (Table 30, FIG. 32) showed that the besylate had good crystallinity. TGA and DSC were tested for the besylate salt which was prepared from THF and recrystallized in ethyl acetate. DSC results showed that the melting point of besylate was 173.25° C. (onset), and TGA showed that there was no significant weight loss from room temperature to 120° C. The result of 1H-NMR showed that the molar ratio of besylate should be 1:1.

TABLE 30
Peak Search Report (33 Peaks, Max P/N = 7.7)
[6-Besylate-THF-EtOAc.RAW] 6-Besylate-THF-EA
PEAK: 23-pts/Parabolic Filter, Threshold = 1.0, Cutoff = 4.0%, BG = 3/1.0,
Peak-Top = Summit

| 2-Theta | d(?) | BG | Height | I % | Area | I % | FWHM |
|---|---|---|---|---|---|---|---|
| 6.638 | 13.304 | 62 | 120 | 36.6 | 2013 | 12.5 | 0.285 |
| 7.758 | 11.3857 | 55 | 101 | 30.8 | 2104 | 13 | 0.354 |
| 9.638 | 9.1689 | 47 | 113 | 34.5 | 2679 | 16.6 | 0.403 |
| 10.319 | 8.5653 | 58 | 72 | 22 | 1601 | 9.9 | 0.378 |
| 11.86 | 7.4559 | 50 | 44 | 13.4 | 1019 | 6.3 | 0.394 |
| 12.538 | 7.0539 | 57 | 77 | 23.5 | 2076 | 12.9 | 0.458 |
| 13.759 | 6.4307 | 66 | 234 | 71.3 | 7420 | 45.9 | 0.539 |
| 14.457 | 6.1217 | 70 | 122 | 37.2 | 4324 | 26.8 | 0.603 |
| 15.56 | 5.6902 | 70 | 168 | 51.2 | 3246 | 20.1 | 0.328 |
| 17.24 | 5.1394 | 70 | 268 | 81.7 | 7600 | 47.1 | 0.482 |
| 17.842 | 4.9671 | 123 | 307 | 93.6 | 16151 | 100 | 0.894 |
| 18.38 | 4.8231 | 125 | 245 | 74.7 | 11019 | 68.2 | 0.765 |
| 19.264 | 4.6038 | 139 | 135 | 41.2 | 4633 | 28.7 | 0.583 |
| 19.562 | 4.5342 | 147 | 207 | 63.1 | 6575 | 40.7 | 0.54 |
| 20.799 | 4.2672 | 146 | 328 | 100 | 9023 | 55.9 | 0.468 |
| 21.921 | 4.0513 | 121 | 141 | 43 | 4749 | 29.4 | 0.573 |
| 22.2 | 4.001 | 115 | 177 | 54 | 4776 | 29.6 | 0.459 |
| 23.599 | 3.7669 | 103 | 315 | 96 | 8406 | 52 | 0.454 |
| 24.4 | 3.6451 | 217 | 191 | 58.2 | 4003 | 24.8 | 0.356 |
| 25.079 | 3.5479 | 172 | 128 | 39 | 2037 | 12.6 | 0.271 |
| 26.238 | 3.3937 | 115 | 121 | 36.9 | 2315 | 14.3 | 0.325 |
| 26.957 | 3.3048 | 112 | 86 | 26.2 | 2697 | 16.7 | 0.533 |
| 27.602 | 3.229 | 87 | 123 | 37.5 | 2834 | 17.5 | 0.392 |
| 28.94 | 3.0827 | 69 | 91 | 27.7 | 2540 | 15.7 | 0.475 |
| 29.287 | 3.047 | 109 | 65 | 19.8 | 2540 | 15.7 | 0.664 |
| 29.745 | 3.001 | 122 | 36 | 11 | 1871 | 11.6 | 0.884 |
| 30.094 | 2.9671 | 130 | 34 | 10.4 | 953 | 5.9 | 0.477 |
| 32.018 | 2.793 | 82 | 72 | 22 | 1669 | 10.3 | 0.394 |
| 36.579 | 2.4546 | 73 | 37 | 11.3 | 1159 | 7.2 | 0.533 |
| 37.197 | 2.4152 | 73 | 29 | 8.8 | 995 | 6.2 | 0.583 |
| 40.867 | 2.2064 | 75 | 39 | 11.9 | 1235 | 7.6 | 0.538 |
| 41.216 | 2.1885 | 76 | 44 | 13.4 | 1227 | 7.6 | 0.474 |
| 43.177 | 2.0935 | 79 | 45 | 13.7 | 955 | 5.9 | 0.361 |

### Example 8: HBr Polymorph of Compound 1a

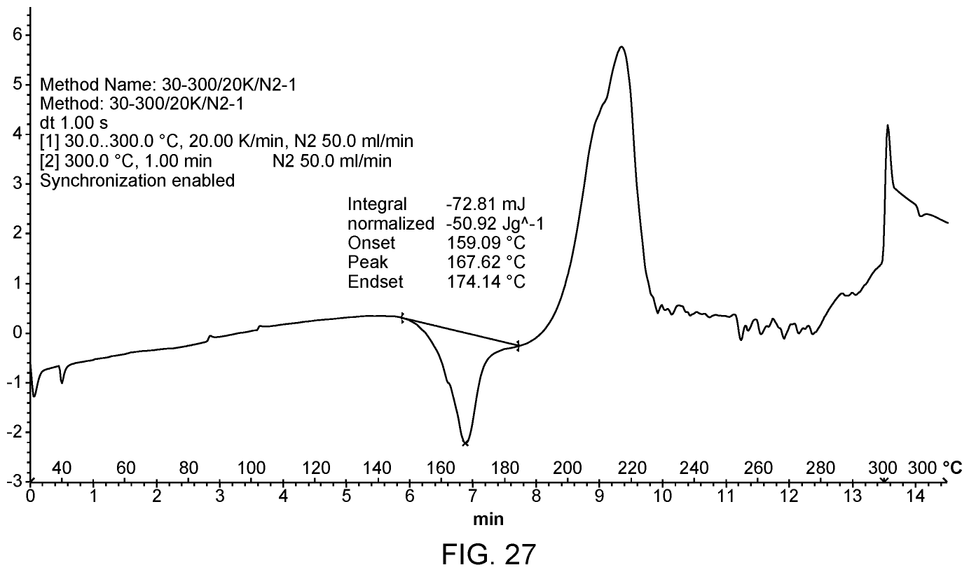

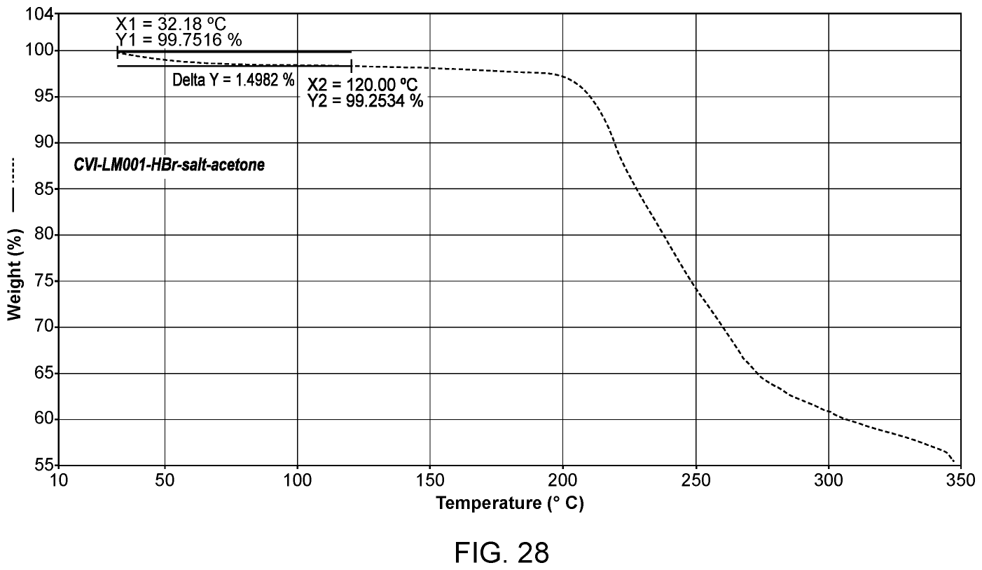

Preparation of crystalline HBr polymorph: Hydrobromide salt was prepared and from a variety of solvent systems (e.g., THF and acetone). However, the crystallinity of hydrobromide salt was poor based on the XRPD results (see FIG. 26 and Table 31 below). Hydrobromide obtained from acetone system was used for TGA and DSC test (FIGS. 27, 28) DSC showed that the melting point of hydrobromide salt was 159.09° C. (onset), and the weight loss of TGA from RT to 120° C. was 1.50%, indicating that there was residual water in the hydrobromide.

TABLE 31
Peak Search Report (15 Peaks, Max P/N = 7.2)
[2-HBr-salt-acetone.RAW] 2-HBr-salt-acetone
PEAK: 23-pts/Parabolic Filter, Threshold = 1.0, Cutoff = 10.0%, BG = 3/1.0,
Peak-Top = Summit

| 2-Theta | d(?) | BG | Height | I % | Area | I % | FWHM |
|---|---|---|---|---|---|---|---|
| 5.416 | 16.3022 | 80 | 52 | 15.4 | 847 | 10.3 | 0.277 |
| 9.706 | 9.105 | 62 | 50 | 14.8 | 846 | 10.2 | 0.288 |
| 9.981 | 8.8547 | 60 | 36 | 10.7 | 1187 | 14.4 | 0.561 |
| 13.496 | 6.5553 | 79 | 53 | 15.7 | 1040 | 12.6 | 0.334 |
| 15.555 | 5.6918 | 159 | 81 | 24 | 1594 | 19.3 | 0.335 |
| 16.339 | 5.4205 | 148 | 130 | 38.6 | 2288 | 27.7 | 0.299 |
| 18.614 | 4.7628 | 128 | 46 | 13.6 | 1001 | 12.1 | 0.37 |
| 18.913 | 4.6882 | 120 | 64 | 19 | 2474 | 30 | 0.657 |
| 21.471 | 4.1352 | 155 | 41 | 12.2 | 1528 | 18.5 | 0.634 |
| 22.982 | 3.8665 | 171 | 39 | 11.6 | 886 | 10.7 | 0.386 |
| 24.5 | 3.6304 | 207 | 337 | 100 | 8255 | 100 | 0.416 |
| 25.735 | 3.4589 | 248 | 68 | 20.2 | 1203 | 14.6 | 0.301 |
| 26.381 | 3.3756 | 208 | 130 | 38.6 | 4161 | 50.4 | 0.544 |
| 29.172 | 3.0587 | 116 | 38 | 11.3 | 1054 | 12.8 | 0.472 |
| 29.637 | 3.0118 | 117 | 51 | 15.1 | 1065 | 12.9 | 0.355 |

### Example 9: Tosylate Polymorph of Compound 1a

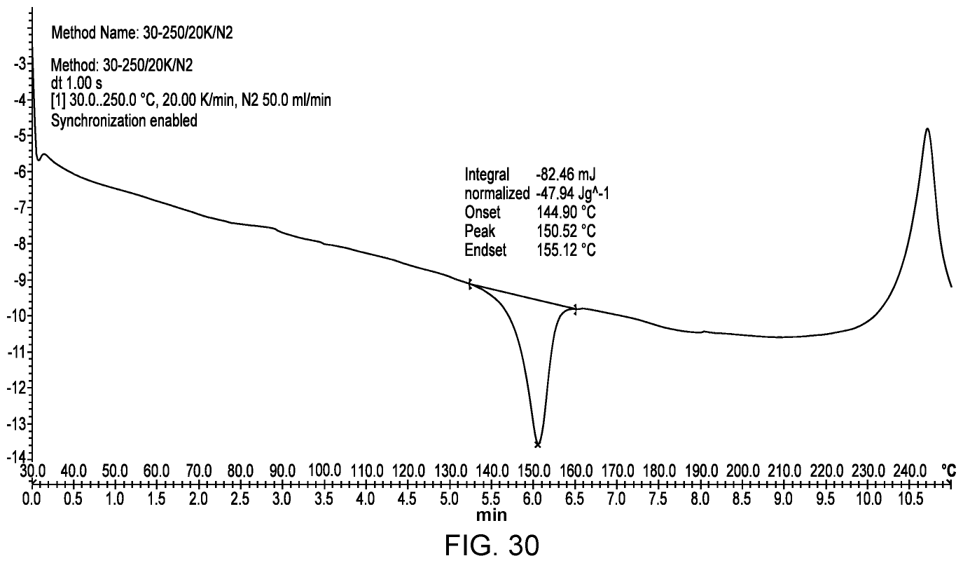

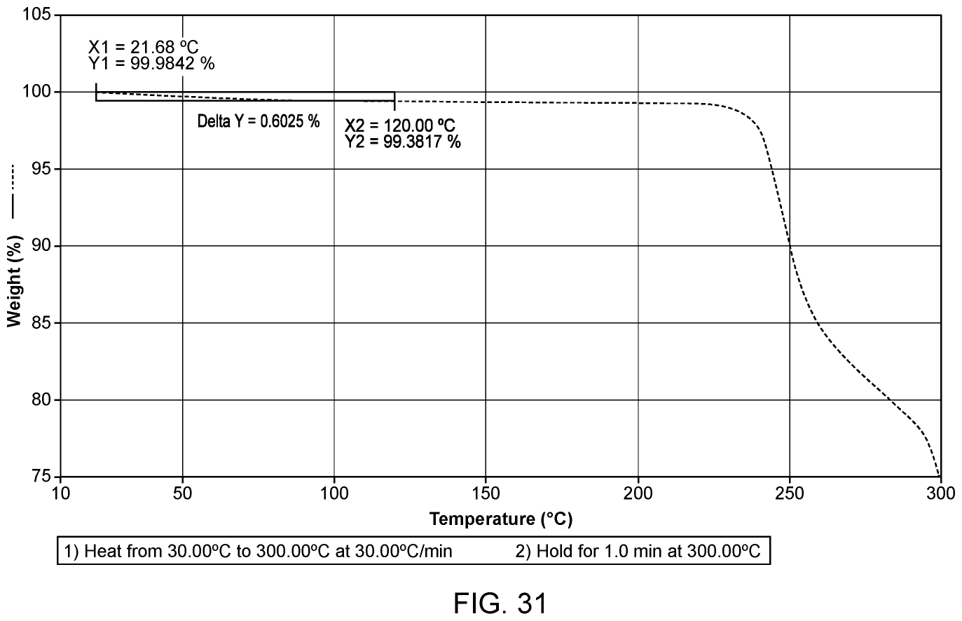

Preparation of crystalline tosylate polymorph: In the small-scale of salt screening, tosylate was obtained by recrystallization by slurry method in ethyl acetate. XRPD results (Table 32, FIG. 29) showed that tosylate had good crystallinity. DSC results (FIG. 30) showed that the melting point of tosylate prepared from MEK system and recrystallized in ethyl acetate was 144.90° C. (onset), which was a little lower. TGA (FIG. 31) result showed that there was almost no significant weight loss from room temperature to 120° C. ¹H-NMR result showed that the molar ratio of tosylate was 1:1 (base: acid).

TABLE 32
Peak Search Report (40 Peaks, Max P/N = 14.2)
[7-Tosilate-MEK-EtOAc.RAW] 7-Tosilate-MEK-EtOAc
PEAK: 23-pts/Parabolic Filter, Threshold = 1.0, Cutoff = 4.0%, BG = 3/1.0,
Peak-Top = Summit

| 2-Theta | d(?) | BG | Height | I % | Area | I % | FWHM |
|---|---|---|---|---|---|---|---|
| 6.381 | 13.8406 | 61 | 111 | 12.5 | 1282 | 9.2 | 0.196 |
| 9.241 | 9.5617 | 54 | 286 | 32.2 | 3942 | 28.3 | 0.234 |
| 9.859 | 8.9642 | 60 | 320 | 36 | 3561 | 25.6 | 0.189 |
| 11.883 | 7.4413 | 56 | 184 | 20.7 | 2112 | 15.2 | 0.195 |
| 12.547 | 7.049 | 68 | 64 | 7.2 | 1081 | 7.8 | 0.287 |
| 12.863 | 6.8764 | 60 | 202 | 22.7 | 4089 | 29.4 | 0.344 |
| 13.362 | 6.6207 | 64 | 524 | 59 | 6534 | 47 | 0.212 |
| 14.241 | 6.2142 | 61 | 379 | 42.7 | 5315 | 38.2 | 0.238 |
| 14.843 | 5.9634 | 60 | 142 | 16 | 1896 | 13.6 | 0.227 |
| 16.56 | 5.3487 | 86 | 364 | 41 | 5468 | 39.3 | 0.255 |
| 17.401 | 5.0922 | 85 | 647 | 72.9 | 7598 | 54.6 | 0.2 |
| 18.661 | 4.751 | 77 | 375 | 42.2 | 8736 | 62.8 | 0.396 |
| 19.463 | 4.557 | 120 | 134 | 15.1 | 1033 | 7.4 | 0.131 |
| 20.059 | 4.423 | 79 | 461 | 51.9 | 13906 | 100 | 0.513 |
| 20.281 | 4.375 | 156 | 460 | 51.8 | 10035 | 72.2 | 0.371 |
| 21.14 | 4.1991 | 143 | 671 | 75.6 | 9365 | 67.3 | 0.237 |
| 22.521 | 3.9447 | 100 | 74 | 8.3 | 1612 | 11.6 | 0.37 |
| 23.139 | 3.8407 | 98 | 448 | 50.5 | 6586 | 47.4 | 0.25 |
| 24.141 | 3.6835 | 90 | 888 | 100 | 13766 | 99 | 0.264 |
| 25.4 | 3.5037 | 95 | 391 | 44 | 5776 | 41.5 | 0.251 |
| 25.8 | 3.4503 | 106 | 126 | 14.2 | 2936 | 21.1 | 0.396 |
| 26.078 | 3.4141 | 119 | 119 | 13.4 | 3027 | 21.8 | 0.432 |
| 26.605 | 3.3477 | 116 | 72 | 8.1 | 951 | 6.8 | 0.225 |
| 26.999 | 3.2998 | 100 | 128 | 14.4 | 2344 | 16.9 | 0.311 |
| 28.28 | 3.1531 | 95 | 289 | 32.5 | 3410 | 24.5 | 0.201 |
| 28.944 | 3.0822 | 100 | 48 | 5.4 | 932 | 6.7 | 0.33 |
| 29.379 | 3.0376 | 95 | 73 | 8.2 | 1236 | 8.9 | 0.288 |
| 30.199 | 2.9569 | 83 | 181 | 20.4 | 3033 | 21.8 | 0.285 |
| 31.221 | 2.8624 | 68 | 100 | 11.3 | 1822 | 13.1 | 0.31 |
| 32.516 | 2.7513 | 66 | 66 | 7.4 | 624 | 4.5 | 0.161 |
| 33.12 | 2.7026 | 59 | 69 | 7.8 | 1620 | 11.6 | 0.399 |
| 33.659 | 2.6605 | 61 | 101 | 11.4 | 1521 | 10.9 | 0.256 |
| 36.037 | 2.4902 | 51 | 95 | 10.7 | 3259 | 23.4 | 0.583 |
| 36.499 | 2.4597 | 60 | 38 | 4.3 | 702 | 5 | 0.314 |
| 38.437 | 2.3401 | 56 | 34 | 3.8 | 739 | 5.3 | 0.37 |
| 40.349 | 2.2335 | 64 | 50 | 5.6 | 835 | 6 | 0.284 |
| 41.118 | 2.1935 | 75 | 37 | 4.2 | 733 | 5.3 | 0.337 |
| 43.32 | 2.0869 | 65 | 55 | 6.2 | 1534 | 11 | 0.474 |
| 44.887 | 2.0177 | 58 | 50 | 5.6 | 1114 | 8 | 0.379 |
| 47.356 | 1.9181 | 49 | 51 | 5.7 | 908 | 6.5 | 0.303 |

### Example 10: Maleate Polymorph of Compound 1a

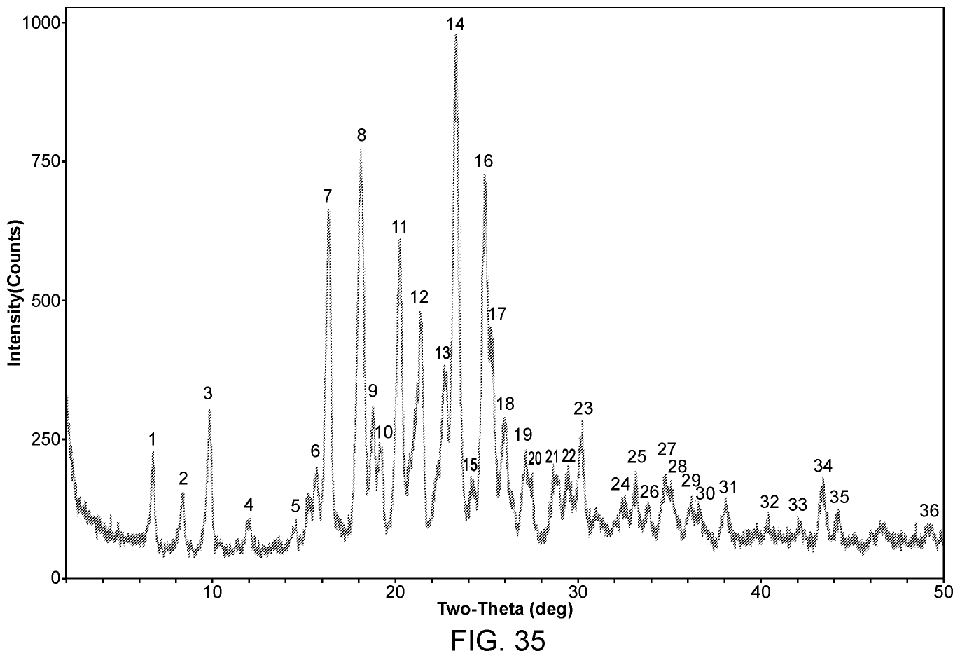

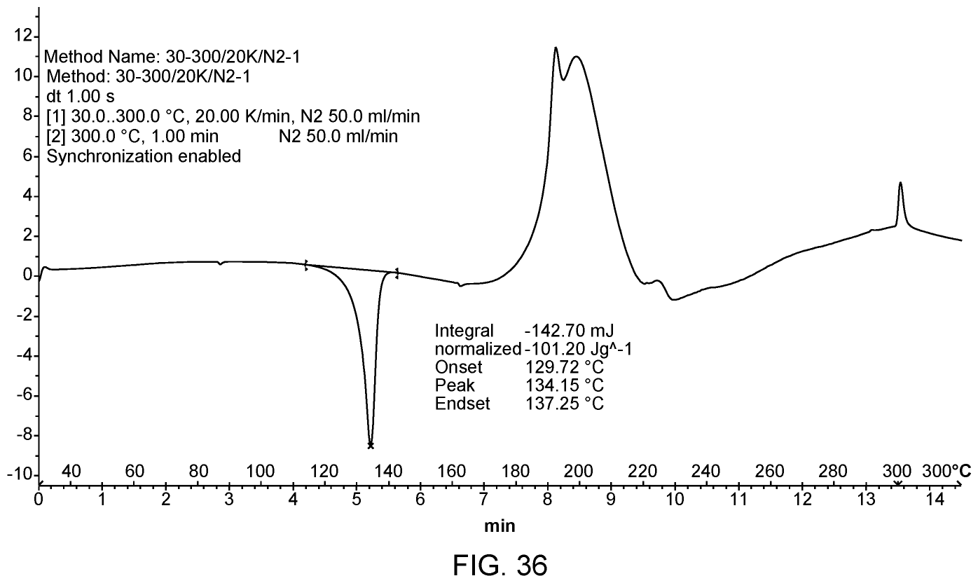

Preparation of crystalline maleate polymorph: The small-scale study demonstrated it was difficult to obtain maleate salt. Maleate salt was obtained by recrystallization from acetone and ethyl acetate by slurry method. The results of XRPD (Table 33, FIG. 35) showed that maleate had good crystallinity. DSC results (FIG. 36) showed that the melting point of maleic acid was 129.72° C. TGA showed that there was almost no obvious weight loss from room temperature to 120° C. (See FIG. 37.) 1H-NMR results showed that the molar ratio of maleate to compound should be 1:1 (base: acid).

TABLE 33
Peak Search Report (36 Peaks, Max P/N = 13.7)
[Maleate-Acetone-EtOAc.RAW] Maleate-Acetone-EtOAc
PEAK: 23-pts/Parabolic Filter, Threshold = 1.0, Cutoff = 2.0%, BG = 3/1.0,
Peak-Top = Summit

| 2-Theta | d(?) | BG | Height | I % | Area | I % | FWHM |
|---|---|---|---|---|---|---|---|
| 6.779 | 13.0281 | 61 | 179 | 20.4 | 2343 | 13.5 | 0.223 |
| 8.437 | 10.472 | 49 | 113 | 12.9 | 2026 | 11.7 | 0.305 |
| 9.861 | 8.9624 | 47 | 273 | 31.1 | 4772 | 27.5 | 0.297 |
| 12.058 | 7.3339 | 51 | 61 | 6.9 | 1133 | 6.5 | 0.316 |
| 14.578 | 6.0711 | 65 | 45 | 5.1 | 425 | 2.4 | 0.161 |
| 15.7 | 5.6399 | 87 | 123 | 14 | 3848 | 22.2 | 0.532 |
| 16.36 | 5.4137 | 107 | 593 | 67.5 | 9993 | 57.5 | 0.286 |
| 18.121 | 4.8915 | 86 | 730 | 83 | 15372 | 88.5 | 0.358 |
| 18.8 | 4.7162 | 95 | 231 | 26.3 | 4536 | 26.1 | 0.334 |
| 19.276 | 4.6008 | 95 | 153 | 17.4 | 3683 | 21.2 | 0.409 |
| 20.259 | 4.3796 | 151 | 493 | 56.1 | 8152 | 46.9 | 0.281 |
| 21.361 | 4.1562 | 131 | 375 | 42.7 | 7992 | 46 | 0.362 |
| 22.68 | 3.9175 | 146 | 258 | 29.4 | 7594 | 43.7 | 0.5 |
| 23.302 | 3.8143 | 153 | 879 | 100 | 17369 | 100 | 0.336 |
| 24.237 | 3.6691 | 140 | 46 | 5.2 | 352 | 2 | 0.13 |
| 24.9 | 3.573 | 165 | 601 | 68.4 | 14454 | 83.2 | 0.409 |
| 25.259 | 3.5229 | 148 | 322 | 36.6 | 10338 | 59.5 | 0.546 |
| 25.964 | 3.4289 | 159 | 145 | 16.5 | 2437 | 14 | 0.286 |
| 27.12 | 3.2853 | 90 | 152 | 17.3 | 3552 | 20.5 | 0.397 |
| 27.461 | 3.2453 | 85 | 115 | 13.1 | 2660 | 15.3 | 0.393 |
| 28.86 | 3.091 | 114 | 80 | 9.1 | 1801 | 10.4 | 0.383 |
| 29.319 | 3.0437 | 131 | 67 | 7.6 | 817 | 4.7 | 0.207 |
| 30.217 | 2.9553 | 115 | 185 | 21 | 2846 | 16.4 | 0.262 |
| 32.383 | 2.7624 | 105 | 47 | 5.3 | 478 | 2.8 | 0.173 |
| 33.14 | 2.701 | 100 | 102 | 11.6 | 2291 | 13.2 | 0.382 |
| 33.8 | 2.6497 | 94 | 50 | 5.7 | 540 | 3.1 | 0.184 |
| 34.758 | 2.5788 | 83 | 115 | 13.1 | 3576 | 20.6 | 0.529 |
| 35.061 | 2.5572 | 82 | 102 | 11.6 | 3577 | 20.6 | 0.596 |
| 36.157 | 2.4822 | 81 | 75 | 8.5 | 1865 | 10.7 | 0.423 |
| 36.637 | 2.4508 | 72 | 58 | 6.6 | 2463 | 14.2 | 0.722 |
| 38.02 | 2.3648 | 71 | 79 | 9 | 1270 | 7.3 | 0.273 |
| 40.384 | 2.2316 | 73 | 49 | 5.6 | 737 | 4.2 | 0.256 |
| 42.105 | 2.1443 | 70 | 38 | 4.3 | 679 | 3.9 | 0.304 |
| 43.36 | 2.0851 | 70 | 120 | 13.7 | 2321 | 13.4 | 0.329 |
| 44.204 | 2.0472 | 69 | 61 | 6.9 | 755 | 4.3 | 0.21 |
| 49.332 | 1.8457 | 66 | 34 | 3.9 | 603 | 3.5 | 0.301 |

### Example 11: Polymorph Physical Properties: Solubilities and Purities

About 5 mg of seven kinds of salts were weighed into each vial, and 2 mL of medium was added, respectively. The medium included water, SGF and FaSSIF. All samples were stirred at 25° C. for 24 hours. After that, the sample was taken out, centrifuged at 12000 rpm for 10 min, and the supernatant was diluted at an appropriate concentration to determine its solubility by HPLC (shown below). HPLC condition were as shown below. The results indicated that the solubility of sulfate in water and SGF was better than that of other salts.

TABLE 34
HPLC Condition for the solubility measurement

| Chromatographic | Waters Sunfire C18, 150*4.6 mm, 5 μm | | |
|---|---|---|---|
| Column | | | |
| Mobile Phase | A: 0.1% TFA in water | | |
| | B: 0.1% TFA in MeOH | | |
| | Time(min) | Phase A | Phase B |
| Gradient elution | 0 | 80 | 20 |
| | 8 | 55 | 45 |
| | 22 | 45 | 55 |
| | 40 | 25 | 75 |

| | |
|---|---|
| Flow Rate | 40° C. |
| Column Temperature | 1 mL/min |
| Injection volume | 10 μL |
| Stop Time | 50 min |
| Detection Wavelength | 278 nm |

TABLE 35
Solubility results of different salts of compound 1a

| | | Solubility |
|---|---|---|
| Salts | Medium | (mg/mL) |
| HCl-salt | Water | 0.129 |
| | SGF | 0.112 |
| | FaSSIF | 0.002 |
| HBr-salt | Water | 0.163 |
| | SGF | 0.156 |
| | FaSSIF | 0.002 |
| sulfate | Water | 1.370 |
| | SGF | 0.496 |
| | FaSSIF | 0.002 |
| Tosylate | Water | 0.411 |
| | SGF | 0.399 |
| | FaSSIF | <0.001 |
| Mesylate | Water | 0.145 |
| | SGF | 0.218 |
| | FaSSIF | 0.002 |
| Besylate | Water | 0.110 |
| | SGF | 0.214 |
| | FaSSIF | 0.001 |
| Maleate | Water | 0.086 |
| | SGF | 0.221 |
| | FaSSIF | 0.001 |

About 5 mg of sample was weighed into a glass bottle, and 2.5 mL of methanol was added in to dissolve it, and the concentration of sample solution was 2 mg/mL. The HPLC conditions were the same as the solubility test method. The chemical purity and relative substance are in Table 36.

TABLE 36
HPLC purity

| | | | RT | 5.44 | 9.58 | 10.70 | 11.94 | 12.52 | 13.09 |
|---|---|---|---|---|---|---|---|---|---|
| Salts | Purity % | TRS % | RRT | 0.38 | 0.68 | 0.76 | 0.84 | 0.89 | 0.93 |
| HCl-salt | 99.45 | 0.55 | \ | \ | \ | 0.10 | \ | \ | \ |
| HBr-salt | 97.66 | 2.34 | \ | \ | \ | 0.18 | 0.04 | 0.17 | 0.14 |
| Sulfate | 99.47 | 0.53 | \ | \ | \ | \ | \ | \ | 0.04 |
| Tosylate | 87.29 | 12.71 | \ | 0.62 | 0.11 | 0.51 | \ | \ | 0.10 |
| Mesylate | 99.69 | 0.31 | \ | \ | \ | \ | \ | \ | \ |
| Besylate | 99.63 | 0.37 | \ | \ | \ | \ | \ | \ | \ |
| Maleate | 96.70 | 3.30 | \ | \ | \ | 0.87 | 0.08 | 0.12 | 0.12 |
| | | | 15.05 | 15.80 | 17.38 | 18.26 | 19.86 | 21.93 | 27.23 |
| Salts | Purity % | TRS % | 1.06 | 1.12 | 1.23 | 1.29 | 1.40 | 1.55 | 1.92 |
| HCl-salt | 99.45 | 0.55 | \ | 0.46 | \ | \ | \ | \ | \ |
| HBr-salt | 97.66 | 2.34 | \ | 1.68 | \ | \ | \ | 0.13 | \ |
| Sulfate | 99.47 | 0.53 | \ | 0.26 | 0.07 | \ | 0.07 | 0.11 | \ |
| Tosylate | 87.29 | 12.71 | \ | 10.31 | \ | 0.37 | 0.45 | \ | 0.23 |
| Mesylate | 99.69 | 0.31 | \ | 0.24 | 0.08 | \ | \ | \ | \ |
| Besylate | 99.63 | 0.37 | \ | 0.30 | 0.08 | \ | \ | \ | \ |
| Maleate | 96.70 | 3.30 | 0.79 | 1.33 | \ | \ | \ | \ | \ |

RT: Retention time;
RRT: Relative retention time;
TRS: Total related substances

The results show chemical purity of hydrochloride salt, sulfate, mesylate and besylate was better was better than the other salt forms. A summary of physical properties for the salt polymorphs of the present Examples is set forth in Table 37.

TABLE 37
Physical Properties of Crystalline Salt Polymorphs

| | | TGA weight | | | |
|---|---|---|---|---|---|
| | Melting | loss (from | | | |
| | point | RT to | Solubility | | |
| Salt | (Onset) | 120° C.) | (mg/mL) | | Stability |
| hydrochloride | 198.36° C. | 0.09% | Water | 0.129 | |
| | | | SGF | 0.112 | |
| | | | FaSSIF | 0.002 | |
| Hydrobromide | 159.09° C. | 1.50% | Water | 0.163 | N/A |
| | | | SGF | 0.156 | |
| | | | FaSSIF | 0.002 | |
| Sulfate | 216.89° C. | 0.12% | Water | 1.370 | N/A |
| | | | SGF | 0.496 | |
| | | | FaSSIF | 0.002 | |
| Mesylate | 145.72° C. | 1.11% | Water | 0.145 | N/A |
| | | | SGF | 0.218 | |
| | | | FaSSIF | 0.002 | |
| Besylate | 173.25° C. | 0.24% | Water | 0.110 | N/A |
| | | | SGF | 0.214 | |
| | | | FaSSIF | 0.001 | |
| Tosylate | 144.90° C. | 0.60% | Water | 0.411 | N/A |
| | | | SGF | 0.399 | |
| | | | FaSSIF | <0.001 | |
| Maleate | 129.72° C. | 0.44% | Water | 0.086 | N/A |
| | | | SGF | 0.221 | |
| | | | FaSSIF | 0.001 | |

Based on the characterization results of various salt polymorphs in the Examples above, it was determined that the melting points of the hydrochloride, sulfate, and besylate polymorphs are relatively high. The sulfate salt was generally more soluble than other polymorphs, but the HCl salt showed it to be the most stable of the polymorphs.

### REFERENCES

1. Al-Allaf F A, Coutelle C, Waddington S N, David A L, Harbottle R, Themis M (2010). “LDLR-Gene therapy for familial hypercholesterolaemia: problems, progress, and perspectives”. Int Arch Med. 3: 36

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

1. A crystalline compound selected from the crystalline hydrochloride, hydrobromide, sulfate, mesylate, besylate, tosylate, or maleate salt of the compound of formula 1a or formula 1b,

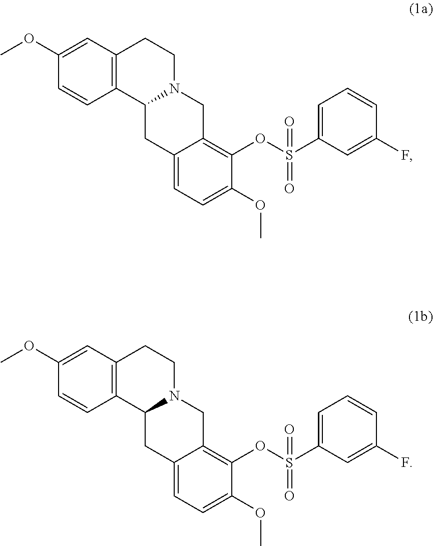 [SMILES: COc1ccc2c(c1)CCN1Cc3c(ccc(OC)c3OS(=O)(=O)c3cccc(F)c3)C[C@H]21 ; COc1ccc2c(c1)CCN1Cc3c(ccc(OC)c3OS(=O)(=O)c3cccc(F)c3)C[C@@H]21]

2. The crystalline compound of claim 1, which is the HCl salt polymorph of formula I or formula II: 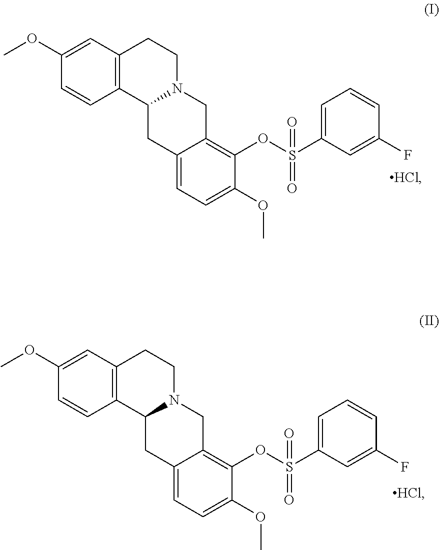
characterized by an X-ray powder diffraction pattern comprising the peaks, expressed in degrees 2θ, of 15.88±0.3, 25.00±0.3, and 20.38±0.3.

3. The crystalline polymorph of claim 2 further comprising the peaks, expressed in degrees 2θ, of 17.64±0.3 and 27.58±0.3.

4. The crystalline polymorph of claim 3 further comprising the peaks, expressed in degrees 2θ, of 24.28±0.3 and 18.54±0.3.

5. The crystalline polymorph of claim 4 further comprising the peaks, expressed in degrees 2θ, of 14.20±0.3 and 6.84±0.3.

6. The crystalline polymorph of claim 5 further comprising the peaks, expressed in degrees 2θ, of 22.02±0.3 and 19.64±0.3.

7. The crystalline polymorph of claim 2, wherein the X-ray powder diffraction (XRPD) pattern is substantially as shown in FIG. 1A or 1B.

8. The crystalline polymorph of claim 2, wherein the crystalline polymorph comprises less than about 0.02% water by mass.

9. The crystalline polymorph of claim 2, wherein the crystalline polymorph has a Differential Scanning calorimetry thermogram onset at about 198° C.

10. The crystalline compound of claim 1, which is the mesylate salt polymorph of formula III or formula IV: 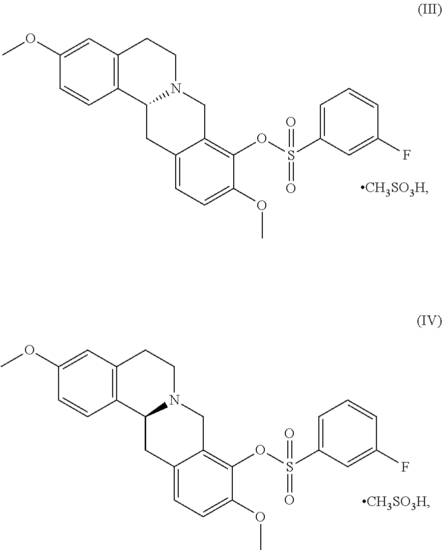
characterized by an X-ray powder diffraction pattern comprising the peaks, expressed in degrees 2θ, of 7.099±0.3, 19.921±0.3, and 8.501±0.3.

11. The crystalline polymorph of claim 10 further comprising the peaks, expressed in degrees 2θ, of 16.917±0.3 and 19.255±0.3.

12. The crystalline polymorph of claim 11 further comprising the peaks, expressed in degrees 2θ, of 35.361±0.3 and 14.601±0.3.

13. The crystalline polymorph of claim 12 further comprising the peaks, expressed in degrees 2θ, of 32.099±0.3 and 28.441±0.3.

14. The crystalline polymorph of claim 13 further comprising the peaks, expressed in degrees 2θ, of 21.076±0.3 and 29.719±0.3.

15. The crystalline polymorph of claim 14, wherein the X-ray powder diffraction (XRPD) pattern is substantially as shown in FIG. 22.

16. The crystalline polymorph of claim 15, wherein the crystalline polymorph has a Differential Scanning calorimetry thermogram onset at about 113° C.

17. The crystalline compound of claim 1 which is the sulfate salt polymorph of formula IX or formula X: 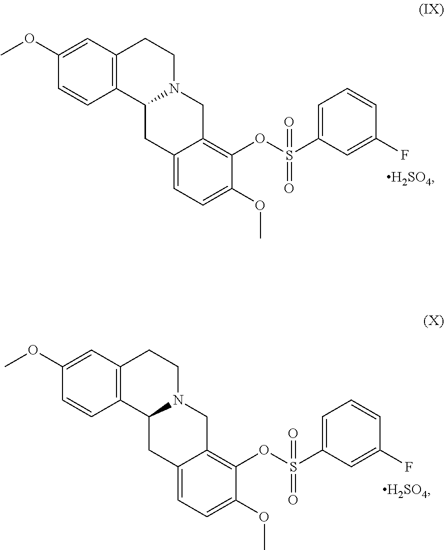
characterized by an X-ray powder diffraction pattern comprising the peaks, expressed in degrees 2θ, of 14.46±0.3, 22.82±0.3, 24.72±0.3.

18. The crystalline polymorph of claim 17 further comprising the peaks, expressed in degrees 2θ, of 14.08±0.3, 17.32±0.3, 26.48±0.3.

19. The crystalline polymorph of claim 18 further comprising the peaks, expressed in degrees 2θ, of 12.278±0.3, 17.9±0.3, 21.42±0.3, 25.159±0.3.

20. The crystalline polymorph of claim 19 further comprising the peaks, expressed in degrees 2θ, of 13.6±0.3, 20.022±0.3, 20.478±0.3, 21.758±0.3.

21. The crystalline polymorph of claim 20, wherein the crystalline polymorph has a Differential Scanning calorimetry thermogram onset at about 217° C.

22. A pharmaceutical composition comprising a pharmaceutically effective amount of the crystalline polymorph of claim 1, and a carrier.

23. A method for producing the crystalline polymorph of claim 2, the method comprising:
contacting a freebase compound of formula V or formula VI: 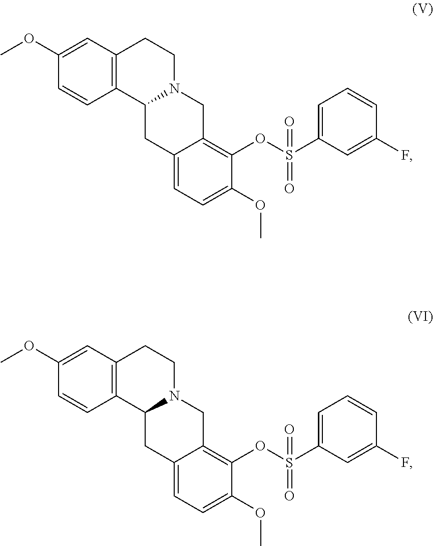 [SMILES: COc1ccc2c(c1)CCN1Cc3c(ccc(OC)c3OS(=O)(=O)c3cccc(F)c3)C[C@H]21 ; COc1ccc2c(c1)CCN1Cc3c(ccc(OC)c3OS(=O)(=O)c3cccc(F)c3)C[C@@H]21]
dissolved in a solvent, with HCl and crystallizing the crystalline polymorph from the solvent.

24. The method of claim 23, wherein the solvent comprises ethanol (EtOH).

25. The method of claim 23 further comprising heating the solvent with the dissolved freebase compound and HCl then cooling the solvent to crystalize the crystalline polymorph.

26. A method for producing the crystalline polymorph of claim 10, the method comprising:
crystallizing a salt of a freebase compound of formula V or formula VI with CH₃SO₃H, from a solvent:

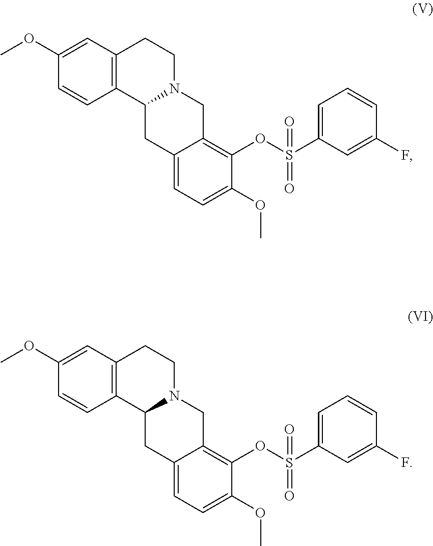 [SMILES: COc1ccc2c(c1)CCN1Cc3c(ccc(OC)c3OS(=O)(=O)c3cccc(F)c3)C[C@H]21 ; COc1ccc2c(c1)CCN1Cc3c(ccc(OC)c3OS(=O)(=O)c3cccc(F)c3)C[C@@H]21]

27. The method of claim 26, wherein the solvent comprises isopropyl alcohol.

28. The method of claim 26, wherein the method comprises contacting the compound of formula V or formula VI, dissolved in a solvent, with CH₃SO₃H.

29. The method of claim 23, wherein the freebase compound is obtained from chiral resolution of a racemate of formula VII:

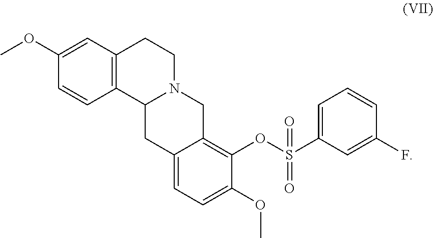 [SMILES: COc1ccc2c(c1)CCN1Cc3c(ccc(OC)c3OS(=O)(=O)c3cccc(F)c3)CC21]

30. The method of claim 29, wherein the chiral resolution of the racemate comprises chromatography with chiral stationary phase, contacting the racemate with chiral reagent, or entrainment and crystallization.

31. A method of treating or preventing dyslipidemia in a subject in need thereof, comprising administering an effective amount of the crystalline polymorph of claim 1, to the subject.

32. The method of claim 31, wherein the dyslipidemia comprises hyperlipidemia.

33. The method of claim 32, wherein the hyperlipidemia comprises hypercholesterolemia or hyperglyceridemia.

34. The method of claim 33, wherein the dyslipidemia comprises hyperlipoproteinemia.
